(19) United States
(12) Patent Application Publication
(10) Pub. No.: US 2024/0209355 A1
(43) Pub. Date: Jun. 27, 2024

(54) NUCLEIC ACID BINDING PROTEIN FORMULATIONS AND USES THEREOF

(71) Applicant: Spotlight Therapeutics, Hayward, CA (US)

(72) Inventors: Aaron Cantor, Pacifica, CA (US); Benjamin G. Gowen, San Leandro, CA (US); Mark W. Knuth, Hayward, CA (US)

(21) Appl. No.: 18/339,859

(22) Filed: Jun. 22, 2023

Related U.S. Application Data
(63) Continuation of application No. PCT/US2021/073105, filed on Dec. 23, 2021.
(60) Provisional application No. 63/130,170, filed on Dec. 23, 2020.
(60) Provisional application No. 63/130,185, filed on Dec. 23, 2020.

(51) Int. Cl. C12N 15/11; C12N 5/00; C12N 9/22; C12N 15/85

(52) U.S. Cl. CPC C12N 15/111; C12N 5/0018; C12N 9/22; C12N 15/85; C12N 2310/20; C12N 2500/12; C12N 2500/34

(57) ABSTRACT

Provided herein are methods and compositions relating to stable aqueous formulations, cell mediums, and ex vivo editing methods for gene editing by site-directed modifying polypeptides.

### RELATED APPLICATIONS

This application claims priority to U.S. Provisional Application No. 63/130,185, filed on Dec. 23, 2020, and U.S. Provisional Application No. 63/130,170 filed on Dec. 23, 2020. The contents of the foregoing applications are incorporated by reference herein in their entirety.

### SEQUENCE LISTING

The instant application contains a Sequence Listing which has been submitted electronically in ASCII format, and which is hereby incorporated by reference in its entirety. Said ASCII copy, created on Dec. 23, 2021, is named S106638_1110WO_SL_ST25.txt, and is 1,015 bytes in size.

### BACKGROUND OF INVENTION

Buffer conditions are an important consideration for the storage stability and activity of ribonucleoproteins (RNPs) comprising a nucleic acid-guided nuclease complexed with a guide RNA, such as Cas9:guide RNA complexes. RNPs are prone to aggregation, especially in physiological conditions (e.g., neutral pH and low salt concentrations), which limits their enzymatic activity and alters their cell penetrating properties (Nguyen et al. Nature biotechnology, 38.1 (2020): 44-49; D'Astolfo, et al., Cell, 161, 674-690 (2015)). Moreover, salt concentration and overall solution tonicity of an RNP formulation can impact a cell's ability to uptake proteins (D'Astolfo, et al., Cell, 161, 674-690 (2015)). Additionally in some RNP production schemes, it is possible to produce samples that contain free guide RNA and/or nucleic acid-guided nuclease in addition to the properly formed RNP. Accordingly, there is need for formulations that maintain RNPs in a stable, un-aggregated state during storage. There is additionally a need for methods to produce and detect formulations having efficient RNP formation and reduced levels of free gRNA.

### FIELD OF INVENTION

Described herein are methods and compositions related to stable aqueous formulations and ex vivo editing mediums for site-directed modifying polypeptides.

### SUMMARY OF INVENTION

Provided herein are stable aqueous formulations for site-directed modifying polypeptides, including Targeted Active Gene Editing (TAGE) agents. The stable aqueous formulations provided herein maintain improved site-directed modifying polypeptide stability (e.g., reduced protein aggregation) relative to the site-directed modifying polypeptide stability observed in standard buffers, such as PBS. Further provided herein are ex vivo editing methods, and related compositions thereof, for modifying a nucleic acid with a site-directed modifying polypeptide in a cell ex vivo.

In one aspect, provided herein is a stable aqueous formulation comprising at least 100 mM of a salt, at least 3% w/v of a sugar or a polyol, and a site-directed modifying polypeptide that recognizes a nucleic acid, wherein the formulation has a pH of about 5 to 8.

In some embodiments, the formulation comprises a reduced level of aggregates of the site-directed modifying polypeptide relative to a reference level as detected by UV/Vis absorbance spectroscopy. In certain embodiments, the reference level is the level of aggregates of the TAGE agent in PBS. In some embodiments, the UV/Vis absorbance spectroscopy is performed at an absorbance of 340 nm. In some embodiments, the level of aggregates of the site-directed modifying polypeptide is determined by Size Exclusion Chromatography (SEC) resin.

In some embodiments, the site-directed modifying polypeptide remains stable after being subjected to two freeze-thaw cycles.

In some embodiments, the site-directed modifying polypeptide remains stable after being subjected to three, four, or five freeze-thaw cycles.

In some embodiments, the site-directed modifying polypeptide is stable during storage at about 4° C. for at least about 4 weeks, at least about 3 months, at least about 6 months, at least about 9 months, or at least about 1 year.

In some embodiments, the site-directed modifying polypeptide is stable during storage at about 22° C. for at least about 4 weeks, at least about 3 months, at least about 6 months, at least about 9 months, or at least about 1 year.

In some embodiments, the site-directed modifying polypeptide is stable during storage at about −20° C. or colder for at least about 4 weeks; at least about 3 months, at least about 6 months, at least about 9 months, at least about 1 year, at least about 2 years, at least about 3 years, or at least about 4 years.

In some embodiments, the site-directed modifying polypeptide has increased stability during storage at about −20° C., 4° C., and/or 22° C. relative to the site-directed modifying polypeptide stored in PBS buffer.

In some embodiments, the sugar is sucrose or trehalose.

In some embodiments, the sugar is sucrose.

In some embodiments, the polyol is a sugar alcohol.

In some embodiments, the sugar alcohol is selected from the group consisting of erythritol, xylitol, sorbitol, mannitol, and inositol.

In some embodiments, the polyol is glycerol.

In some embodiments, the polyol is propylene glycol.

In some embodiments, the stable aqueous formulation comprises at least about 4%, at least about 5% w/v, at least about 7.5% w/v, at least about 10% w/v, at least about 12.5% w/v, or at least about 15% w/v of the polyol or the sugar.

In some embodiments, the stable aqueous formulation comprises 3% w/v to 15% w/v of the polyol or the sugar. In some embodiments, the stable aqueous formulation comprises 5% w/v to 10% w/v of the polyol or the sugar. In some embodiments, the stable aqueous formulation comprises 10% w/v to 15% w/v of the polyol or the sugar. In some embodiments, the stable aqueous formulation comprises 3% w/v to 6% w/v of the polyol or the sugar. In some embodiments, the stable aqueous formulation comprises 6% w/v to 8% w/v of the polyol or the sugar. In some embodiments, the stable aqueous formulation comprises 8% w/v to 10% w/v of the polyol or the sugar. In some embodiments, the stable aqueous formulation comprises 10% w/v to 12% w/v of the polyol or the sugar. In some embodiments, the stable aqueous formulation comprises 12% w/v to 14% w/v of the polyol or the sugar. In some embodiments, the stable aqueous formulation comprises 14% w/v to 16% w/v of the polyol or the sugar.

In some embodiments, the salt is a sodium salt or a potassium salt. In certain embodiments, the sodium salt is NaCl. In certain embodiments, the potassium salt is KCl.

In some embodiments, the stable aqueous formulation comprises at least about 150 mM, at least about 175 mM, at least about 185 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, or at least about 450 mM of the salt.

In some embodiments, the stable aqueous formulation comprises 150 mM to 500 mM of the salt. In some embodiments, the stable aqueous formulation comprises 185 mM to 450 mM of the salt. In some embodiments, the stable aqueous formulation comprises 150 mM to 250 mM of the salt. In some embodiments, the stable aqueous formulation comprises 250 mM to 500 mM of the salt. In some embodiments, the stable aqueous formulation comprises 150 mM to 200 mM of the salt. In some embodiments, the stable aqueous formulation comprises 200 mM to 250 mM of the salt. In some embodiments, the stable aqueous formulation comprises 250 mM to 300 mM of the salt. In some embodiments, the stable aqueous formulation comprises 300 mM to 350 mM of the salt. In some embodiments, the stable aqueous formulation comprises 350 mM to 400 mM of the salt. In some embodiments, the stable aqueous formulation comprises 400 mM to 450 mM of the salt. In some embodiments, the stable aqueous formulation comprises 450 mM to 500 mM of the salt. In some embodiments, the stable aqueous formulation comprises a free amino acid. In some embodiments, the free amino acid is histidine, serine, threonine, or arginine.

In some embodiments, the stable aqueous formulation comprises at least about 5 mM, at least about 10 mM, at least about 25 mM, at least about 50 mM, at least about 75 mM, at least about 100 mM, at least about 150 mM, or at least about 200 mM of the free amino acid.

In some embodiments, the stable aqueous formulation comprises 1-250 mM of the free amino acid. In some embodiments, the stable aqueous formulation comprises 1-25 mM of the free amino acid. In some embodiments, the stable aqueous formulation comprises 25-50 mM of the free amino acid. In some embodiments, the stable aqueous formulation comprises 50-75 mM of the free amino acid. In some embodiments, the stable aqueous formulation comprises 75-100 mM of the free amino acid. In some embodiments, the stable aqueous formulation comprises 75-100 mM of the free amino acid. In some embodiments, the stable aqueous formulation comprises 100-150 mM of the free amino acid. In some embodiments, the stable aqueous formulation comprises 150-200 mM of the free amino acid.

In some embodiments, the formulation has a pH of about 5.5-7.5.

In some embodiments, the formulation comprises 0.1-100 μM of the site-directed modifying polypeptide.

In some embodiments, the formulation further comprises a surfactant.

In some embodiments, the formulation does not comprise phosphate buffered saline (PBS).

In some embodiments, the formulation comprises at least 150 mM of a salt and at least 5% w/v of a sugar.

In some embodiments, the formulation comprises at least 200 mM of a salt and at least 5% w/v of a sugar.

In some embodiments, the sugar is sucrose and the salt is NaCl.

In some embodiments, the formulation comprises at least 5% sucrose (w/v), at least 200 mM NaCl, at least 10 mM histidine, and at least 50 mM arginine.

In some embodiments, the formulation comprises 5% sucrose (w/v), 300 mM NaCl, 20 mM L-histidine, and 100 mM arginine.

In some embodiments, the stable aqueous formulation further comprises chloroquine (e.g., in combination with the salt and the sugar and/or polyol).

In some embodiments, the site-directed modifying polypeptide that recognizes a nucleic acid is a nucleic acid-guided nuclease.

In some embodiments, the nucleic acid-guided nuclease is an RNA-guided nuclease.

In some embodiments, the RNA-guided nuclease is a Class 2 Cas polypeptide. In certain embodiments, the Class 2 Cas polypeptide is a Type II Cas polypeptide. In certain embodiments, the Type II Cas polypeptide is Cas9. In certain embodiments, the Class 2 Cas polypeptide is a Type V Cas polypeptide. In some embodiments, the Type V Cas polypeptide is Cas12.

In some embodiments, the stable aqueous formulation comprises a guide nucleic acid (gNA), wherein the gNA and the nucleic acid-guided nuclease form a nucleoprotein.

In some embodiments, the guide nucleic acid is a guide RNA (gRNA), the nucleic acid-guided nuclease is an RNA-guided nuclease, and the gRNA and RNA-guided nuclease form a ribonucleoprotein (RNP).

In some embodiments, the gRNA is a single guide RNA (sgRNA) or a cr:trRNA.

In some embodiments, the gRNA is a re-folded gRNA that is a capable of eluting as a single peak from a Size Exclusion Chromatography resin when the gRNA is not complexed to the site-directed modifying polypeptide.

In some embodiments, the site-directed modifying polypeptide further comprises a cell targeting agent, thereby forming a targeted active gene editing (TAGE) agent.

In some embodiments, the cell targeting agent is a ligand, a cell penetrating peptide, or an antigen-binding polypeptide.

In some embodiments, the ligand binds to an extracellular cell membrane-bound molecule or protein.

In some embodiments, the antigen binding polypeptide is an antibody, an antigen-binding portion of an antibody, or an antibody mimetic.

In some embodiments, the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, a versabody, an aptamer, or a peptidic molecule.

In some embodiments, the antigen-binding portion of the antibody is a nanobody, a domain antibody, an scFv, an Fab, a diabody, a BiTE, a diabody, a DART, a minibody, an F(ab′)2, or an intrabody.

In some embodiments, the antibody is an intact antibody or a bispecific antibody.

In some embodiments, the antigen binding polypeptide binds to an extracellular cell membrane-bound molecule or protein.

In another aspect, provided herein is a pharmaceutical composition comprising a stable aqueous formulation provided herein.

In yet another aspect, provided herein is a method of modifying a nucleic acid in a target cell, the method comprising contacting the target cell with a stable aqueous formulation provided herein.

In some embodiments, the target cell is a eukaryotic cell. In certain embodiments, the eukaryotic cell is a mammalian cell. In some embodiments, the mammalian cell is a mouse cell, a non-human primate cell, or a human cell. In some embodiments, the mammalian cell is a hematopoietic stem cell (HSC), a neutrophil, a T cell, a B cell, a dendritic cell, a macrophage, or a fibroblast.

In some embodiments, the nucleic acid is in the genome of the target cell.

In some embodiments, the nucleic acid is a target gene in the genome of the target cell.

In some embodiments, the method is effective to modify expression of the target gene.

In another aspect, provided herein is a method of modifying a nucleic acid sequence within a cell in a mammalian subject, the method comprising administering to the subject a stable aqueous formulation herein or a pharmaceutical composition provided herein, such that the nucleic acid sequence of the cell is modified.

In another aspect, provided herein is a method of modifying a nucleic acid sequence within a cell in a mammalian subject, the method comprising locally administering to the subject a stable aqueous formulation herein or a pharmaceutical composition provided herein, such that the nucleic acid sequence of the cell is modified.

In some embodiments, the stable aqueous formulation or the pharmaceutical composition is administered to the subject by intramuscular injection, intraosseous injection, intraocular injection, intratumoral injection, or intradermal injection.

In some embodiments, the mammalian subject is a human subject.

In another aspect, provided herein is an aqueous formulation for modifying a nucleic acid comprising a site-directed modifying polypeptide that recognizes the nucleic acid, and at least 1 μM chloroquine. In some embodiments, the aqueous formulation comprises at least 10 μM chloroquine. In some embodiments, the aqueous formulation comprises at least 20 μM chloroquine. In some embodiments, the aqueous formulation comprises at least 30 μM chloroquine. In some embodiments, the aqueous formulation comprises at least 50 μM chloroquine. In some embodiments, the aqueous formulation comprises at least 80 μM chloroquine. In some embodiments, the aqueous formulation comprises at least 100 μM chloroquine. In some embodiments, the aqueous formulation comprises 1-200 μM chloroquine. In some embodiments, the aqueous formulation comprises 10-150 μM chloroquine. In some embodiments, the aqueous formulation comprises 25-100 μM chloroquine. In some embodiments, the aqueous formulation comprises 25-50 μM chloroquine. In some embodiments, the aqueous formulation comprises 50-75 μM chloroquine. In some embodiments, the aqueous formulation comprises 75-100 μM chloroquine.

In some embodiments, the aqueous formulation comprises 0.1-100 μM of the site-directed modifying polypeptide.

In some embodiments, the site-directed modifying polypeptide that recognizes the nucleic acid is a nucleic acid-guided nuclease. In some embodiments, the nucleic acid-guided nuclease is an RNA-guided nuclease. In some embodiments, the RNA-guided nuclease is a Class 2 Cas polypeptide. In some embodiments, the Class 2 Cas polypeptide is a Type II Cas polypeptide. In certain embodiments, the Type II Cas polypeptide is Cas9. In some embodiments, the Class 2 Cas polypeptide is a Type V Cas polypeptide. In certain embodiments, the Type V Cas polypeptide is Cas 12.

In some embodiments, the aqueous formulation a guide nucleic acid (gNA), wherein the gNA and the nucleic acid-guided nuclease form a nucleoprotein. In some embodiments, the guide nucleic acid is a guide RNA (gRNA), the nucleic acid-guided nuclease is an RNA-guided nuclease, and the gRNA and the RNA-guided nuclease form a ribonucleoprotein (RNP). In some embodiments, the gRNA is a single guide RNA (sgRNA) or a cr:trRNA.

In some embodiments, the site-directed modifying polypeptide further comprises a cell targeting agent, thereby forming a targeted active gene editing (TAGE) agent.

In some embodiments, the cell targeting agent is a ligand, a cell penetrating peptide, and antigen-binding polypeptide, or a combination thereof.

In some embodiments, the antigen-binding polypeptide is an antibody, an antigen-binding portion of an antibody, or an antibody mimetic.

In some embodiments, the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, a versabody, an aptamer, or a peptidic molecule.

In some embodiments, the antigen-binding portion of the antibody is a nanobody, a domain antibody, an scFv, an Fab, a diabody, a BiTE, a diabody, a DART, a minibody, an F(ab′)2, or an intrabody.

In some embodiments, the antibody is an intact antibody or a bispecific antibody.

In some embodiments, the antigen binding polypeptide binds to an extracellular cell membrane-bound molecule or protein.

In some embodiments, the ligand binds to an extracellular cell membrane-bound molecule or protein.

In another aspect, provided herein is a pharmaceutical composition comprising an aqueous formulation provided herein.

In yet another aspect, provided herein is a method of modifying a nucleic acid in a target cell, the method comprising contacting the target cell with an aqueous formulation provided herein.

In some embodiments, the target cell is a eukaryotic cell. In certain embodiments, the eukaryotic cell is a mammalian cell. In some embodiments, the mammalian cell is a mouse cell, a non-human primate cell, or a human cell. In some embodiments, the mammalian cell is a hematopoietic stem cell (HSC), a neutrophil, a T cell, a B cell, a dendritic cell, a macrophage, or a fibroblast.

In some embodiments, the nucleic acid is in the genome of the target cell.

In some embodiments, the nucleic acid is a target gene in the genome of the target cell.

In some embodiments, the method is effective to modify expression of the target gene.

In another aspect, provided herein is a method of modifying a nucleic acid sequence within a cell in a mammalian subject, the method comprising administering to the subject an aqueous formulation provided herein or a pharmaceutical composition provided herein, such that the nucleic acid sequence of the cell is modified.

In another aspect, provided herein is a method of modifying a nucleic acid sequence within a cell in a mammalian subject, the method comprising locally administering to the subject an aqueous formulation herein or a pharmaceutical composition provided herein, such that the nucleic acid sequence of the cell is modified.

In some embodiments, the aqueous formulation or the pharmaceutical composition is administered to the subject by intramuscular injection, intraosseous injection, intraocular injection, intratumoral injection, or intradermal injection.

In some embodiments, the mammalian subject is a human subject.

In one aspect, also provided herein is a method for modifying a nucleic acid in a cell ex vivo, the method comprising culturing the cell in a cell medium, and contacting the cell in the cell medium with a site directed-modifying polypeptide that recognizes the nucleic acid, wherein the cell medium comprises a salt, and a polyol and/or a sugar, such that the nucleic acid in the cell is modified.

In another aspect, provided herein is a method of increasing genome editing in a population of cells ex vivo, the method comprising culturing the population of cells in a cell medium, and contacting the population of cells in the cell medium with a site directed modifying polypeptide that recognizes a nucleic acid in the genome of a cell in the population of cells, wherein the cell medium comprises a salt and polyol and/or a sugar, such that genome editing is increased in the population of cells.

In yet another aspect, provided herein is a method for modifying a nucleic acid in a cell ex vivo, the method comprising contacting the cell in a cell medium with a site directed-modifying polypeptide that recognizes the nucleic acid, wherein the cell medium comprises a salt, and a polyol and/or a sugar, such that the nucleic acid in the cell is modified.

In a further aspect, provided herein is a method of increasing genome editing in a population of cells ex vivo, the method comprising contacting the population of cells in a cell medium with a site directed modifying polypeptide that recognizes a nucleic acid in the genome of a cell in the population of cells, wherein the cell medium comprises a salt and polyol and/or a sugar, such that genome editing is increased in the population of cells.

In some embodiments, the cell medium is supplemented to have a concentration of salt, a polyol, or a sugar, or a combination thereof.

In some embodiments, the salt, the polyol, or the sugar is added to the cell medium prior to contacting the cells with the site directed modifying polypeptide; is added concurrently with contacting the cells with the site directed modifying polypeptide; or is added after contacting the cells with the site directed modifying polypeptide.

In some embodiments, the cell medium has an effective concentration of the salt, the polyol, or the sugar, or a combination thereof prior to contacting the cells with the site directed modifying polypeptide, such that a supplement of the salt, the polyol, or the sugar is not provided.

In some embodiments, the polyol is a sugar alcohol. In some embodiments, the sugar alcohol is selected from the group consisting of erythritol, xylitol, mannitol, and inositol.

In some embodiments, the sugar alcohol is xylitol.

In some embodiments, the polyol is glycerol.

In some embodiments, polyol is propylene glycol.

In some embodiments, the sugar is sucrose.

In some embodiments, the salt is a sodium salt or a potassium salt. In some embodiments, the sodium salt is NaCl. In some embodiments, the potassium salt is KCl.

In some embodiments, the cell medium comprises at least about 150 mM, at least about 175 mM, at least about 185 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, or at least about 450 mM of the salt.

In some embodiments, the cell medium comprises 100 mM to 500 mM of the salt. In some embodiments, the cell medium comprises 185 mM to 450 mM of the salt. In some embodiments, the cell medium comprises 100 mM to 250 mM of the salt. In some embodiments, the cell medium comprises 250 mM to 500 mM of the salt. In some embodiments, the cell medium comprises 150 mM to 200 mM of the salt. In some embodiments, the cell medium comprises 200 mM to 250 mM of the salt. In some embodiments, the cell medium comprises 250 mM to 300 mM of the salt. In some embodiments, the cell medium comprises 300 mM to 350 mM of the salt. In some embodiments, the cell medium comprises 350 mM to 400 mM of the salt. In some embodiments, the cell medium comprises 400 mM to 450 mM of the salt. In some embodiments, the cell medium comprises 450 mM to 500 mM of the salt.

In some embodiments, the cell medium comprises at least about 0.2 M, at least about 0.4 M, at least about 0.8 M, at least about 1.2 M, at least about 1.6 M, at least about 2.0 M, at least about 2.4 M, at least about 2.6 M of the polyol or the sugar.

In some embodiments, the cell medium comprises 0.2 M to 2.6 M of the polyol or the sugar. In some embodiments, the cell medium comprises 0.4 M to 2.4 M of the polyol or the sugar. In some embodiments, the cell medium comprises 0.4 M to 1 M of the polyol or the sugar. In some embodiments, the cell medium comprises 1 M to 2.5 M of the polyol or the sugar. In some embodiments, the cell medium comprises 0.2 M to 0.5 M of the polyol or the sugar. In some embodiments, the cell medium comprises 0.5 M to 1 M of the polyol or the sugar. In some embodiments, the cell medium comprises 1 M to 1.5 M of the polyol or the sugar. In some embodiments, the cell medium comprises 1.5 M to 2 M of the polyol or the sugar. In some embodiments, the cell medium comprises 2 M to 2.5 M of the polyol or the sugar.

In some embodiments, the cell medium comprises at least about 4% w/v, at least about 5% w/v, at least about 7.5% w/v, at least about 10% w/v, at least about 12.5% w/v, or at least about 15% w/v of the polyol or the sugar. In some embodiments, the cell medium comprises 3% w/v to 15% w/v of the polyol or the sugar. In some embodiments, the cell medium comprises 4% w/v to 8% w/v of the polyol or the sugar. In some embodiments, the cell medium comprises 8% w/v to 12% w/v of the polyol or the sugar. In some embodiments, the cell medium comprises 5% w/v to 10% w/v of the polyol or the sugar. In some embodiments, the cell medium comprises 10% w/v to 15% w/v of the polyol or the sugar. In some embodiments, the cell medium comprises 3% w/v to 6% w/v of the polyol or the sugar. In some embodiments, the cell medium comprises 6% w/v to 8% w/v of the polyol or the sugar. In some embodiments, the cell medium comprises 8% w/v to 10% w/v of the polyol or the sugar. In some embodiments, the cell medium comprises 10% w/v to 12% w/v of the polyol or the sugar. In some embodiments, the cell medium comprises 12% w/v to 14% w/v of the polyol or the sugar. In some embodiments, the cell medium comprises 14% w/v to 16% w/v of the polyol or the sugar.

In some embodiments, cell medium further comprises chloroquine (e.g., in combination with the salt and the polyol and/or the sugar).

In some embodiments, cell medium is supplemented to have a concentration of chloroquine (e.g., in combination with the salt and the polyol and/or the sugar).

In some embodiments, cell medium is supplemented to have a concentration of chloroquine (e.g., in combination with the salt and the polyol and/or the sugar).

In another aspect, provided herein is a method for modifying a nucleic acid in a cell ex vivo, the method comprising: culturing the cell in a cell medium, and contacting the cell in the cell medium with a site directed-modifying polypeptide that recognizes the nucleic acid, wherein the cell medium comprises chloroquine, such that the nucleic acid in the cell is modified.

In another aspect, provided herein is a method of increasing genome editing in a population of cells ex vivo, the method comprising culturing the population of cells in a cell medium, and contacting the population of cells in the cell medium with a site directed modifying polypeptide that recognizes a nucleic acid in the genome of a cell in the population of cells, and wherein the cell medium comprises chloroquine, such that genome editing is increased in the population of cells.

In another aspect, provided herein is a method for modifying a nucleic acid in a cell ex vivo, the method comprising contacting the cell in a cell medium with a site directed-modifying polypeptide that recognizes the nucleic acid, wherein the cell medium comprises chloroquine, such that the nucleic acid in the cell is modified.

In another aspect, provided herein is a method of increasing genome editing in a population of cells ex vivo, the method comprising contacting the population of cells in a cell medium with a site directed modifying polypeptide that recognizes a nucleic acid in the genome of a cell in the population of cells, and wherein the cell medium comprises chloroquine, such that genome editing is increased in the population of cells.

In some embodiments, the cell medium is supplemented to have a concentration of chloroquine.

In some embodiments, the chloroquine is added to the cell medium prior to contacting the cells with the site directed modifying polypeptide; is added concurrently with contacting the cells with the site directed modifying polypeptide; or is added after contacting the cells with the site directed modifying polypeptide.

In some embodiments, the cell medium has an effective concentration of chloroquine prior to contacting the cells with the site directed modifying polypeptide, such that a supplement is not provided.

In some embodiments, the cell medium comprises at least 10 μM chloroquine. In some embodiments, the cell medium comprises at least 30 μM chloroquine. In some embodiments, the cell medium comprises at least 50 μM chloroquine. In some embodiments, the cell medium comprises at least 80 μM chloroquine. In some embodiments, the cell medium comprises at least 100 μM chloroquine. In some embodiments, the cell medium comprises 1-200 μM chloroquine. In some embodiments, the cell medium comprises 10-150 μM chloroquine. In some embodiments, the cell medium comprises 25-100 μM chloroquine. In some embodiments, the cell medium comprises 25-50 μM chloroquine. In some embodiments, the cell medium comprises 50-75 μM chloroquine. In some embodiments, the cell medium comprises 75-100 μM chloroquine.

In some embodiments, the site-directed modifying polypeptide that recognizes the nucleic acid is a nucleic acid-guided nuclease. In some embodiments, the nucleic acid-guided nuclease is an RNA-guided nuclease. In some embodiments, the RNA-guided nuclease is a Class 2 Cas polypeptide. In some embodiments, the Class 2 Cas polypeptide is a Type II Cas polypeptide. In some embodiments, the Type II Cas polypeptide is Cas9. In some embodiments, the Class 2 Cas polypeptide is a Type V Cas polypeptide. In some embodiments, the Type V Cas polypeptide is Cas 12.

In some embodiments, the site-directed modifying polypeptide further comprise a guide nucleic acid (gNA), wherein the gNA and the nucleic acid-guided nuclease form a nucleoprotein. In some embodiments, the guide nucleic acid is a guide RNA (gRNA), the nucleic acid-guided nuclease is an RNA-guided nuclease, and the gRNA and the RNA-guided nuclease form a ribonucleoprotein. In some embodiments, the gRNA is a single guide RNA (sgRNA) or a cr:trRNA.

In some embodiments, the site-directed modifying polypeptide further comprises a cell targeting agent, thereby forming a targeted active gene editing (TAGE) agent.

In some embodiments, the cell targeting agent is a ligand, a cell penetrating peptide, and antigen-binding polypeptide, or a combination thereof.

In some embodiments, the antigen-binding polypeptide is an antibody, an antigen-binding portion of an antibody, or an antibody mimetic. In some embodiments, the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, a versabody, an aptamer, or a peptidic molecule. In some embodiments, the antigen-binding portion of the antibody is a nanobody, a domain antibody, an scFv, an Fab, a diabody, a BiTE, a diabody, a DART, a minibody, an F(ab′)2, or an intrabody. In some embodiments, the antibody is an intact antibody or a bispecific antibody. In some embodiments, the antigen binding polypeptide binds to an extracellular cell membrane-bound molecule or protein.

In some embodiments, the ligand binds to an extracellular cell membrane-bound molecule or protein.

In some embodiments, the cell or a cell in the population of cells is a eukaryotic cell. In certain embodiments, the eukaryotic cell is a mammalian cell. In some embodiments, the mammalian cell is a mouse cell, a non-human primate cell, or a human cell. In some embodiments, the mammalian cell is a hematopoietic stem cell (HSC), a neutrophil, a T cell, a B cell, a dendritic cell, a macrophage, or a fibroblast.

In some embodiments, the nucleic acid is in the genome of the cell. In some embodiments, the nucleic acid is a target gene in the genome of the target cell. In some embodiments, the method is effective to modify expression of the target gene.

In some embodiments, the cell in the cell medium is contacted with the site-directed modifying polypeptide by co-incubation ex vivo.

In some embodiments, the cell in the cell medium is not contacted with the site-directed modifying polypeptide by nucleofection.

In another aspect, provided herein is a cell medium for ex vivo genome editing of a population of cells, the cell medium comprising an effective amount of: a salt and a polyol and/or a sugar, wherein the effective amount is effective for increasing gene editing in the population of cells relative to a reference level, wherein the reference level is determined according to either the gene editing achieved in the population of cells in the absence of the salt and the polyol and/or the sugar, or the gene editing achieved in the population of cells using an amount of the salt and the polyol and/or the sugar that is less than the effective amount.

In another aspect, provided herein is a cell medium for ex vivo genome editing of a population of cells, the cell medium comprising an effective amount of: a salt, and a polyol and/or a sugar, wherein the effective amount is an amount of the salt and the polyol and/or the sugar that achieves at least 5% editing in the population cells.

In another aspect, provided herein is a cell medium for ex vivo genome editing of a population of cells, the cell medium comprising an effective amount of a salt, and a polyol and/or a sugar, wherein the effective amount is an amount of the salt and the polyol and/or the sugar that achieves greater than 0% editing in the population of cells, and the population of cells are not amenable to electroporation, nucleofection, transfection, and/or transduction.

In some embodiments, the polyol is a sugar alcohol.

In some embodiments, the sugar alcohol is selected from the group consisting of erythritol, xylitol, mannitol, and inositol.

In some embodiments, the sugar alcohol is xylitol.

In some embodiments, the polyol is glycerol.

In some embodiments, the polyol is propylene glycol.

In some embodiments, the sugar is sucrose.

In some embodiments, the salt is a sodium salt or a potassium salt. In some embodiments, the sodium salt is NaCl. In some embodiments, the potassium salt is KCl.

In some embodiments, the cell medium comprises at least about 150 mM, at least about 175 mM, at least about 185 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, or at least about 450 mM of the salt.

In some embodiments, the cell medium comprises 100 mM to 500 mM of the salt. In some embodiments, the cell medium comprises 185 mM to 450 mM of the salt. In some embodiments, the cell medium comprises 100 mM to 250 mM of the salt. In some embodiments, the cell medium comprises 250 mM to 500 mM of the salt. In some embodiments, the cell medium comprises 150 mM to 200 mM of the salt. In some embodiments, the cell medium comprises 200 mM to 250 mM of the salt. In some embodiments, the cell medium comprises 250 mM to 300 mM of the salt. In some embodiments, the cell medium comprises 300 mM to 350 mM of the salt. In some embodiments, the cell medium comprises 350 mM to 400 mM of the salt. In some embodiments, the cell medium comprises 400 mM to 450 mM of the salt. In some embodiments, the cell medium comprises 450 mM to 500 mM of the salt.

In some embodiments, the cell medium comprises at least about 0.2 M, at least about 0.4 M, at least about 0.8 M, at least about 1.2 M, at least about 1.6 M, at least about 2.0 M, at least about 2.4 M, at least about 2.6 M of the polyol or the sugar.

In some embodiments, the cell medium comprises 0.2 M to 2.6 M of the polyol or the sugar. In some embodiments, the cell medium comprises 0.4 M to 2.4 M of the polyol or the sugar. In some embodiments, the cell medium comprises 0.4 M to 1 M of the polyol or the sugar. In some embodiments, the cell medium comprises 1 M to 2.5 M of the polyol or the sugar. In some embodiments, the cell medium comprises 0.2 M to 0.5 M of the polyol or the sugar. In some embodiments, the cell medium comprises 0.5 M to 1 M of the polyol or the sugar. In some embodiments, the cell medium comprises 1 M to 1.5 M of the polyol or the sugar. In some embodiments, the cell medium comprises 1.5 M to 2 M of the polyol or the sugar. In some embodiments, the cell medium comprises 2 M to 2.5 M of the polyol or the sugar.

In some embodiments, the cell medium comprises at least about 4%, at least about 5% w/v, at least about 7.5% w/v, at least about 10% w/v, at least about 12.5% w/v, or at least about 15% w/v of the polyol or the sugar.

In some embodiments, the cell medium comprises 3% w/v to 15% w/v of the polyol or the sugar. In some embodiments, the cell medium comprises 3% w/v to 8% w/v of the polyol or the sugar. In some embodiments, the cell medium comprises 8% w/v to 12% w/v of the polyol or the sugar. In some embodiments, the cell medium comprises 5% w/v to 10% w/v of the polyol or the sugar. In some embodiments, the cell medium comprises 10% w/v to 15% w/v of the polyol or the sugar. In some embodiments, the cell medium comprises 4% w/v to 6% w/v of the polyol or the sugar. In some embodiments, the cell medium comprises 6% w/v to 8% w/v of the polyol or the sugar. In some embodiments, the cell medium comprises 8% w/v to 10% w/v of the polyol or the sugar. In some embodiments, the cell medium comprises 10% w/v to 12% w/v of the polyol or the sugar. In some embodiments, the cell medium comprises 12% w/v to 14% w/v of the polyol or the sugar. In some embodiments, the cell medium comprises 14% w/v to 16% w/v of the polyol or the sugar.

In some embodiments, the cell medium further comprises an effective amount of chloroquine (e.g., in combination with the salt and sugar and/or polyol).

In another aspect, provided herein is a cell medium for ex vivo genome editing of a population of cells, the cell medium comprising an effective amount of chloroquine, wherein the effective amount is effective for increasing gene editing in the population of cells relative to a reference level, wherein the reference level is determined according to either the gene editing achieved in the population of cells in the absence of chloroquine, or the gene editing achieved in the population of cells using an amount of chloroquine that is less than the effective amount.

In another aspect, provided herein is a cell medium for ex vivo genome editing of a population of cells, the cell medium comprising an effective amount of chloroquine, wherein the effective amount is an amount of chloroquine that achieves at least 5% editing in the population cells.

In another aspect, provided herein is a cell medium for ex vivo genome editing of a population of cells, the cell medium comprising an effective amount of chloroquine, wherein the effective amount is an amount of chloroquine that achieves greater than 0% editing in the population of cells, and the population of cells are not amenable to electroporation, nucleofection, transfection, and/or transduction.

In some embodiments, the cell medium comprises at least 10 μM chloroquine. In some embodiments, the cell medium comprises at least 30 μM chloroquine. In some embodiments, the cell medium comprises at least 50 μM chloroquine. In some embodiments, the cell medium comprises at least 80 μM chloroquine. In some embodiments, the cell medium comprises at least 100 μM chloroquine. In some embodiments, the cell medium comprises 1-200 μM chloroquine. In some embodiments, the cell medium comprises 10-150 μM chloroquine. In some embodiments, the cell medium comprises 25-100 μM chloroquine. In some embodiments, the cell medium comprises 25-50 μM chloroquine. In some embodiments, the cell medium comprises 50-75 μM chloroquine. In some embodiments, the cell medium comprises 75-100 μM chloroquine.

In some embodiments, the cell medium further comprises site-directed modifying polypeptide that recognizes a nucleic acid in a cell. In some embodiments, the site-directed modifying polypeptide that recognizes the nucleic acid is a nucleic acid-guided nuclease. In some embodiments, the nucleic acid-guided nuclease is an RNA-guided nuclease. In some embodiments, the RNA-guided nuclease is a Class 2 Cas polypeptide. In some embodiments, the Class 2 Cas polypeptide is a Type II Cas polypeptide. In certain embodiments, the Type II Cas polypeptide is Cas9. In some embodiments, the Class 2 Cas polypeptide is a Type V Cas polypeptide. In certain embodiments, the Type V Cas polypeptide is Cas 12.

In some embodiments, the cell medium further comprises a guide nucleic acid (gNA), wherein the gNA and the nucleic acid-guided nuclease form a nucleoprotein. In some embodiments, the guide nucleic acid is a guide RNA (gRNA), the nucleic acid-guided nuclease is an RNA-guided nuclease, and the gRNA and the RNA-guided nuclease form a ribonucleoprotein. In some embodiments, the gRNA is a single guide RNA (sgRNA) or a cr:trRNA.

In some embodiments, the site-directed modifying polypeptide further comprises a cell targeting agent, thereby forming a targeted active gene editing (TAGE) agent.

In some embodiments, the cell targeting agent is a ligand, a cell penetrating peptide, and antigen-binding polypeptide, or a combination thereof.

In some embodiments, the antigen-binding polypeptide is an antibody, an antigen-binding portion of an antibody, or an antibody mimetic. In some embodiments, the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, a versabody, an aptamer, or a peptidic molecule. In some embodiments, the antigen-binding portion of the antibody is a nanobody, a domain antibody, an scFv, an Fab, a diabody, a BiTE, a diabody, a DART, a minibody, an F(ab′)2, or an intrabody. In some embodiments, the antibody is an intact antibody or a bispecific antibody. In some embodiments, the antigen binding polypeptide binds to an extracellular cell membrane-bound molecule or protein.

In some embodiments, the ligand binds to an extracellular cell membrane-bound molecule or protein.

In some embodiments, the cell medium further comprises the population of cells.

In some embodiments, the cell or a cell in the population of cells is a eukaryotic cell. In some embodiments, the eukaryotic cell is a mammalian cell. In some embodiments, the mammalian cell is a mouse cell, a non-human primate cell, or a human cell. In some embodiments, the mammalian cell is a hematopoietic stem cell (HSC), a neutrophil, a T cell, a B cell, a dendritic cell, a macrophage, or a fibroblast.

In some embodiments, the cell medium further comprises an effective amount of a salt and a polyol and/or a sugar (e.g., in combination with the effective amount of chloroquine).

In a further aspect, provided herein is a stable aqueous formulation comprising at least 100 mM of a salt, at least 3% w/v of a sugar or a sugar alcohol, and a site-directed modifying polypeptide that recognizes a nucleic acid, wherein the formulation has a pH of about 5 to 8.

In some embodiments, the formulation comprises a reduced level of aggregates of the site-directed modifying polypeptide relative to a reference level as detected by UV/Vis absorbance spectroscopy; remains stable after being subjected to at least two freeze-thaw cycles; is stable during storage at about 4° C. for at least about 4 weeks, at least about 3 months, at least about 6 months, at least about 9 months, or at least about 1 year; and/or is stable during storage at about 22° C. for at least about 4 weeks, at least about 3 months, at least about 6 months, at least about 9 months, or at least about 1 year.

In some embodiments, the reference level is the level of aggregates of the site-directed modifying polypeptide in phosphate buffered solution (PBS) as determined by size exclusion chromatography (SEC).

In some embodiments, the sugar is sucrose or trehalose. In some embodiments, the sugar alcohol is selected from the group consisting of glycerol, erythritol, xylitol, sorbitol, mannitol, and inositol.

In some embodiments, the formulation comprises at least about 1% w/v, at least about 2% w/v, at least about 3% w/v, at least about 4% w/v, at least about 5% w/v, at least about 7.5% w/v, at least about 10% w/v, at least about 12.5% w/v, at least about 15% w/v, at least about 20% w/v, at least about 25% w/v, at least about 30% w/v, at least about 35% w/v, at least about 40% w/v, at least about 45% w/v, at least about 50% w/v, or 1%-50% w/v of the sugar alcohol or the sugar.

In some embodiments, the salt is NaCl or KCl.

In some embodiments, the formulation comprises at least about 50 mM, at least about 100 mM, at least about 150 mM, at least about 175 mM, at least about 185 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 500 mM, at least about 750 mM, at least about 1000 mM, at least about 1225 mM, at least about 1500 mM, at least about 1750 mM, at least about 2000 mM, or 50 mM to 2000 mM of the salt.

In some embodiments, the formulation has a pH of about 5.5-7.5 or about 5-8.5.

In some embodiments, the formulation comprises 0.1-100 μM or 0.1-50 μM of the site-directed modifying polypeptide.

In some embodiments, the formulation comprises at least 150 mM of a salt and at least 5% w/v of a sugar.

In some embodiments, the sugar is sucrose and the salt is NaCl.

In some embodiments, the sugar is sucrose and the salt is KCl.

In some embodiments, the sugar alcohol is xylitol and the salt is NaCl.

In some embodiments, the sugar alcohol is xylitol and the salt is KCl.

In some embodiments, the site-directed modifying polypeptide that recognizes a nucleic acid is an RNA-guided nuclease. In some embodiments, the RNA-guided nuclease is a Class 2 Cas polypeptide. In some embodiments, the Class 2 Cas polypeptide is a Type II Cas polypeptide or a Type V Cas polypeptide. In some embodiments, the Type II Cas polypeptide is Cas9 or wherein the Type V Cas polypeptide is Cas12.

In some embodiments, the formulation further comprises a guide nucleic acid (gNA), wherein the gNA and the RNA-guided nuclease form a ribonucleoprotein (RNP). In some embodiments, the gRNA is a single guide RNA (sgRNA) or a cr:trRNA.

In some embodiments, the gRNA is a re-folded gRNA that is a capable of eluting as a single peak from a Size Exclusion Chromatography resin when the gRNA is not complexed to the site-directed modifying polypeptide.

In some embodiments, the site-directed modifying polypeptide further comprises a cell targeting agent, thereby forming a targeted active gene editing (TAGE) agent.

In some embodiments, the cell targeting agent is a ligand, a cell penetrating peptide, or an antigen-binding polypeptide. In some embodiments, the antigen binding polypeptide is an antibody, an antigen-binding portion of an antibody, or an antibody mimetic.

In another aspect, provided herein is a method of modifying a nucleic acid in a target cell, the method comprising contacting the target cell with an aqueous formulation provided herein.

In a further aspect, provided herein is a method for modifying a nucleic acid in a cell ex vivo, the method comprising: contacting at least one cell in a cell medium with a site directed-modifying polypeptide that recognizes a nucleic acid, wherein the at least one cell comprises the nucleic acid, wherein the cell medium comprises an effective amount of a salt and/or a sugar alcohol and/or a sugar, such that the nucleic acid in the at least one cell is modified.

In yet a further aspect, provided herein is a method of achieving genome editing in a population of cells ex vivo, the method comprising contacting a population of cells in a cell medium with a site directed modifying polypeptide that recognizes a nucleic acid in the genome of cells in the population of cells, wherein the cell medium comprises an effective amount of a salt and/or a sugar alcohol and/or a sugar, such that genome editing is achieved in the population of cells.

In some embodiments, the salt, the sugar alcohol or the sugar is added according to one of the following: the salt, the sugar alcohol, or the sugar is added to the cell medium prior to contacting the cells with the site directed modifying polypeptide; the salt, the sugar alcohol, or the sugar is added concurrently with contacting the cells with the site directed modifying polypeptide; or the salt, the sugar alcohol, or the sugar is added after contacting the cells with the site directed modifying polypeptide.

In some embodiments, the cell medium comprises the effective amount of the salt, the sugar alcohol, or the sugar, prior to contacting the cells with the site directed modifying polypeptide.

In some embodiments, the sugar alcohol is selected from the group consisting of erythritol, xylitol, mannitol, and inositol. In some embodiments, the sugar alcohol is glycerol. In some embodiments, the sugar is sucrose. In some embodiments, the sugar alcohol is xylitol.

In some embodiments, the salt is NaCl or KCl.

In some embodiments, the cell medium comprises at least about 50 mM, at least about 100 mM, at least about 150 mM, at least about 175 mM, at least about 185 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, or at least about 450 mM of the salt.

In some embodiments, the cell medium comprises 50 mM to 500 mM, 100 mM to 500 mM of the salt, 185 mM to 450 mM of the salt, 100 mM to 250 mM of the salt, 250 mM to 500 mM of the salt, 150 mM to 200 mM of the salt, 200 mM to 250 mM of the salt, 250 mM to 300 mM of the salt, 300 mM to 350 mM of the salt, 350 mM to 400 mM of the salt, 400 mM to 450 mM of the salt, or 450 mM to 500 mM of the salt.

In some embodiments, the cell medium comprises at least about 0.2 M, at least about 0.4 M, at least about 0.8 M, at least about 1.2 M, at least about 1.6 M, at least about 2.0 M, at least about 2.4 M, at least about 2.6 M of the sugar alcohol or the sugar. In some embodiments, the cell medium comprises 0.2 M to 2.6 M, 0.4 M to 2.4 M, 0.4 M to 1 M, 1 M to 2.5 M, 0.2 M to 0.5 M, 0.5 M to 1 M, 1 M to 1.5 M, 1.5 M to 2 M, or 2 M to 2.5 M, of the sugar alcohol or the sugar.

In some embodiments, the cell medium comprises at least about 1% w/v, at least about 2% w/v, at least about 3% w/v, at least about 4% w/v, at least about 5% w/v, at least about 7.5% w/v, at least about 10% w/v, at least about 12.5% w/v, at least about 15% w/v, at least about 20% w/v, at least about 25% w/v, at least about 30% w/v, at least about 35% w/v, at least about 40% w/v, at least about 45% w/v, or at least about 50% w/v of the sugar alcohol or the sugar.

In some embodiments, the cell medium comprises 4% w/v to 15% w/v, 4% w/v to 8% w/v, 8% w/v to 12%, 5% w/v to 10% w/v, 10% w/v to 15%, or 4% w/v to 6% w/v, 6% w/v to 8% w/v, 8% w/v to 10% w/v, 10% w/v to 12% w/v, 12% w/v to 14% w/v, 14% w/v to 16% w/v, 1% to 50%, 5% to 50%, 10% to 50%, 15% to 50%, 20% to 50%, 25% to 50%, 30% to 50%, 40% to 50, 45% to 50%, 1% to 40%, 5% to 40%, 5% to 35%, 5% to 30%, 5% to 25%, 5% to 20%, 5% to 15%, 5% to 10%, 1% to 30%, 5% to 30%, 10% to 30%, 15% to 30%, 20% to 30%, 25 to 30%, 1% to 20%, 5% to 20%, 10% to 20%, or 15% to 20% of the sugar alcohol or the sugar.

In some embodiments, the site-directed modifying polypeptide is an RNA-guided nuclease. In some embodiments, the RNA-guided nuclease is a Class 2 Cas polypeptide. In some embodiments, the Class 2 Cas polypeptide is a Type II Cas polypeptide or a Type V Cas polypeptide. In some embodiments, the Type II Cas polypeptide is Cas9 or the Type V Cas polypeptide is Cas 12. In some embodiments, the RNA-guided nuclease further comprises a guide nucleic acid (gNA), wherein the gNA and the RNA-guided nuclease form a ribonucleoprotein. In some embodiments, the gRNA is a single guide RNA (sgRNA) or a cr:trRNA.

In some embodiments, the site-directed modifying polypeptide further comprises a cell targeting agent, thereby forming a targeted active gene editing (TAGE) agent. In some embodiments, the cell targeting agent is a ligand, a cell penetrating peptide, an antigen-binding polypeptide, or a combination thereof.

In some embodiments, the antigen-binding polypeptide is an antibody, an antigen-binding portion of an antibody, or an antibody mimetic. In some embodiments, the antigen binding polypeptide binds to an extracellular cell membrane-bound molecule or protein.

In another aspect, provided herein is a method for modifying a nucleic acid in a cell ex vivo, the method comprising contacting at least one cell in a cell medium, with a targeted active gene editing agent (TAGE) that recognizes a nucleic acid in the at least one cell, wherein the TAGE comprises a cell targeting agent and a site-directed modifying polypeptide, and wherein the cell medium comprises an effective amount of a salt and/or a sugar alcohol and/or a sugar, such that the nucleic acid in the cell is modified.

In a further aspect, provided herein is a method of achieving genome editing in a population of cells ex vivo, the method comprising contacting a population of cells in a cell medium, with a targeted active gene editing agent (TAGE) that recognizes a nucleic acid in the genome of cells in the population of cells, wherein the TAGE comprises a cell targeting agent and a site-directed modifying polypeptide, wherein the cell medium comprises an effective amount of a salt and/or a sugar alcohol and/or a sugar, such that genome editing is achieved in the population of cells.

In some embodiments, the TAGE further comprises a guide nucleic acid (gNA). In some embodiments, the gRNA is a single guide RNA (sgRNA) or a cr:trRNA.

In some embodiments, the cell targeting agent is a ligand, a cell penetrating peptide (CPP), an antigen-binding polypeptide, or a combination thereof. In some embodiments, the antigen-binding polypeptide is an antibody, an antigen-binding portion of an antibody, or an antibody mimetic.

In some embodiments, the site-directed modifying polypeptide is an RNA-guided nuclease. In some embodiments, the RNA-guided nuclease is a Class 2 Cas polypeptide. In some embodiments, the Class 2 Cas polypeptide is a Type II Cas polypeptide or a Type V Cas polypeptide. In some embodiments, the Type II Cas polypeptide is Cas9 or the Type V Cas polypeptide is Cas 12.

In some embodiments, the site-directed modifying polypeptide is an RNA-guided nuclease and the cell targeting agent comprises an antibody, or an antigen-binding portion thereof.

In some embodiments, the sugar alcohol is selected from the group consisting of erythritol, xylitol, mannitol, glycerol, and inositol.

In some embodiments, the sugar is sucrose.

In some embodiments, the salt is sodium chloride.

In some embodiments, the sugar is xylitol.

In some embodiments, the salt, the sugar alcohol, or the sugar is added according to one of the following: is added to the cell medium prior to contacting the cells with the TAGE; is added concurrently with contacting the cells with the TAGE; or is added after contacting the cells with the TAGE.

In some embodiments, the cell or a cell in the population of cells is a eukaryotic cell. In some embodiments, the eukaryotic cell is a mammalian cell. In some embodiments, the mammalian cell is a mouse cell, a non-human primate cell, or a human cell. In some embodiments, the mammalian cell is a hematopoietic stem cell (HSC), a neutrophil, a T cell, a B cell, a dendritic cell, a macrophage, or a fibroblast.

In some embodiments, the nucleic acid is in the genome of the cell. In some embodiments, the nucleic acid is a target gene in the genome of the target cell. In some embodiments, the method is effective to modify expression of the target gene. In some embodiments, the cell in the cell medium is contacted with the site-directed modifying polypeptide by co-incubation ex vivo. In some embodiments, the site-directed modifying polypeptide is not internalized into the cell by nucleofection.

In another aspect, provided herein is a method described in the Examples and Figures provided herein.

In a further aspect, provided herein is medium or supplement described in the Examples and Figures provided herein.

### BRIEF DESCRIPTION OF THE DRAWINGS

FIGS. 1A-1C graphically depict UV/Vis absorbance spectra of TAGE agent ribonucleoproteins (RNPs) at different salt and sugar concentrations. Light scattering, as indicated by the sloping baseline, indicates aggregation of the TAGE agent RNPs. FIG. 1A graphically depicts the UV/Vis absorbance spectra of TAGE agents (Cas9(C80A)-2×NLS or 4×NLS-Cas9(C80A)-2×NLS) complexed with one of two guide RNAs (gRNAs) (sgBFP or sgJD98)) following incubation in PG buffer (PBS+10% glycerol) or SH300 buffer (20 mM L-Histidine pH 7.4, 300 mM NaCl, 100 mM L-Arginine, 5% w/v sucrose) for 37 C for 10 minutes (bottom panel). FIG. 1B graphically depicts the UV/Vis absorbance spectra of Cas9(C80A) RNPs (Cas9(C80A):sgBFP gRNA) at different NaCl concentrations (150, 200, 250, 300, and 350 mM), sucrose concentrations (2.5% or 5.0%), and temperatures (22° C. and 37° C.). The graphs are shaded according to their NaCl concentration and separated by their incubation temperature (22° C. on the left plot and 37° C. on the right plot) and sucrose concentration (2.5% sucrose on the top plot and 5% sucrose on the bottom plot). FIG. 1C graphically depicts the UV/Vis absorbance at 340 nm from the spectra in FIG. 1B, at the indicated sucrose concentrations (2.5% or 5% sucrose) and temperatures as a function of NaCl concentration.

FIGS. 2A and 2B depicts the size exclusion chromatography (SEC)-high performance liquid chromatography (HPLC) profile of single guide RNAs (JD298) in HLE buffer, SEC buffer, or 1×RNP buffer. FIG. 2A compares the SEC-HPLC elution profiles of a sgRNA that has been re-folded as compared to gRNA that has not undergone the re-folding process. FIG. 2B graphically depicts the SEC-HPLC elution profiles of sgRNA in SEC running buffer with or without Mg²⁺ and with or without prior sgRNA re-folding.

FIG. 3 graphically depicts size exclusion chromatography (SEC)-high performance liquid chromatography (HPLC) chromatograms (absorbance at 260 nm) for three RNP samples at different molar ratios of Cas9(C80A) (“C80A”) to a sgRNA (sgBFP), as indicated in the figure. The free RNA (top plot) eluted at ˜12 minutes. The C80A:sgBFP RNP eluted at 10.5-10.6 minutes, with a small shoulder at ˜9.2 minutes. A small free RNA peak was visible with a slight molar excess of Cas9 (middle plot), and no free RNA was visible with 2.4-fold molar excess Cas9 (bottom plot).

FIGS. 4A and 4B graphically depict the anion exchange-high performance liquid chromatography profiles of Cas9 at the indicated concentrations (FIG. 4A) or Cas9 RNPs formed from solutions with varying Cas9:guide RNA ratios (FIG. 4B).

FIGS. 5A and 5B graphically depict the results of Cas9(C80A)-2×NLS stability studies. FIG. 5A graphically depicts the results of an in vitro DNA cleavage assay of Cas9 (C80A)-2×NLS RNP pre-treated with the indicated murine serum, plasma, blood, or tumor microenvironment (TME) overtime prior to the in vitro cleavage assay. FIG. 5B graphically depicts the results of an in vitro DNA cleavage assay of Cas9 (C80A)-2×NLS RNP pre-treated with buffers at the indicated pH over time prior to the in vitro cleavage assay.

FIGS. 6A and 6B graphically depict the results of an assay assessing the impact of freeze-thaw cycles on the in vitro DNA cleavage activity of TAGE agent RNPs (4×NLS-Cas9(C80A)-2×NLS (“4×NLS”), Cas9-IL2, or Cas9(C80A)-2×NLS (“C80A”)). FIG. 6A graphically depicts the results of a DNA cleavage assay of the indicated TAGE agent RNPs complexed with different guide RNAs (a single guide RNA (sgRNA), cr:tr, cr_xt:tr, or cr:tr550) after exposure to zero, one, or two freeze-thaw cycles. FIG. 6B graphically depicts the results of a DNA cleavage assay of the indicated TAGE agent RNPs (complexed with a sgRNA) after exposure to zero, one, or two freeze-thaw cycles in PBS with or without 5% glycerol. (FIG. 6B).

FIGS. 7A-7C graphically depict the results of an assay to evaluate the impact of gRNA re-folding on Cas9 solubility and in vitro DNA cleavage activity. FIG. 7A graphically depicts the results of an optical density assay for the turbidity of RNPs (Cas9 complexed with sgRNA) including gRNAs that have (“re-folded”) or have not (“un-folded”) undergone prior re-folding. Cas9 without guide RNA (Apo Cas9) or guide RNA in Tris buffer were evaluated as comparators. FIGS. 7B and 7C graphically depict the results of an in vitro DNA cleavage assay with the indicated TAGE agents (4×NLS-Cas9(C80A)-2×NLS (“4×NLS”), Cas9-IL2 (“C9-IL2”), or Cas9(C80A)-2×NLS (“C80A”)) complexed with sgRNA that have (“re-folded”) or have not (“un-folded”) undergone prior re-folding. RNPs composed of the indicated TAGE agents and re-folded or unfolded gRNA were reconstituted in GF buffer (FIG. 7B) or PBS (FIG. 7C) and assessed for DNA cleavage activity.

FIG. 8 graphically depicts the results of an ex vivo editing assay in which TAGE agents including cell penetrating peptides conjugated to Cas12 were co-incubated with fibroblasts and assessed for editing in different glycerol-containing buffers (1.25% glycerol with cells or 6.25% glycerol with cells). A guide RNA targeting an intron of the mouse Hprt gene was associated with the respective TAGE agents to form ribonucleoproteins, and the ribonucleoproteins were co-incubated with fibroblasts to test for editing. Editing efficiency was measured using a T7 endonuclease I assay. The assessed CPP TAGE agents included Cas12a-Wildtype (“WTCas12a”, 6×HIS-AsCas12a(WT)-2× SV40 NLS), 6×His-AsCas12(wt)-4×NLS-2×NLS (“WTCas12a-4×NLS”), EnCas12a (i.e., 6×His-AsCas12a(E174R/S542R/K548R)-2×NLS), and Cas12a Ultra (IDT).

FIG. 9 graphically depicts the results of an ex vivo editing assay in which TAGE agents including Cas9-2×NLS:sgBFP, 4×NLS-Cas9-2×NLS, Cas9-2×NLS-SpyCatcher-4×NLS, or IL-2[-SpyTag]:Cas9-2×NLS-SpyCatcher-4×NLS were co-incubated with primary human T cells and assessed for editing in buffers including different levels of chloroquine (0 μM, 10 μM, 30 μM, or 100 μM). A guide RNA targeting CD47 was associated with the respective TAGE agents to form ribonucleoproteins, and the ribonucleoproteins were co-incubated with T cells to test for editing. Editing was measured using a phenotypic readout measuring the loss of surface CD47 using flow cytometry.

FIGS. 10A and 10B graphically depict the results of an ex vivo editing assay in which TAGE agents including 4×NLS-Cas9-2×NLS were co-incubated with human T cells and assessed for editing in buffers including different levels of salt (185 mM NaCl, 250 mM NaCl, 300 mM NaCl, or 400 mM NaCl NaCl) and glycerol (1%, 5%, 7.5%, 10%, 12.5%, or 15% w/v glycerol). A guide RNA targeting CD47 was associated with the respective TAGE agents to form ribonucleoproteins, and the ribonucleoproteins were co-incubated with T cells to test for editing. After a 1 hour co-incubation, the cells were washed with cell medium to remove additives and RNPs. Editing was measured using a phenotypic readout measuring the loss of surface CD47 using flow cytometry. The percentage of T cells that were edited under each condition are shown in FIG. 10A. Additionally, the levels of live cells per mL 24 hours after co-incubation in the indicated buffer was assessed for each buffer condition, as shown in FIG. 10B.

FIGS. 11A-11E graphically depict the results of an ex vivo editing assay in which TAGE agents including 4×NLS-Cas9-2×NLS were co-incubated with human T cells and assessed for editing in buffers including salt (NaCl) in combination with different sugars or sugar alcohols (e.g., sucrose, propylene glycol, glycerol, erythritol, xylitol, mannitol, inositol). Buffers having different salt concentrations (185 mM NaCl or 300 mM NaCl) and sugar alcohol or sugar concentrations (0.4 M, 0.8 M, 1.2 M, 1.6 M, 2.0 M, or 2.4 M) were assessed. A guide RNA targeting CD47 was associated with the respective TAGE agents to form ribonucleoproteins, and the ribonucleoproteins were co-incubated with T cells to test for editing. After a 1 hour co-incubation, the cells were washed with cell medium to remove additives and RNPs. Editing was measured using a phenotypic readout measuring the loss of surface CD47 using flow cytometry. The percentage of T cells that were edited under each condition are shown in FIGS. 11A-11C. Additionally, the levels of live cells per mL 24 hours after co-incubation in the indicated buffer was assessed for each buffer condition, as shown in FIGS. 11D and 11E. FIG. 11A depicts the levels of editing and toxicity under the indicated buffer conditions as a function of sugar OH group concentration (M). FIG. 11B depicts the levels of editing and toxicity under the indicated buffer conditions as a function of sugar molar concentration. The levels of editing (FIG. 11C) and toxicity (FIG. 11D) are also depicted separately for each of the experiments with propylene glycol, glycerol, erythritol, xylitol, mannitol, inositol, and sucrose. FIG. 11E depicts the level of editing as a function of cell viability in the indicated buffer conditions.

FIG. 12 graphically depicts the results of an ex vivo editing assay in which TAGE agents including 4×NLS-Cas9-2×NLS or AsCas12a were co-incubated with human T cells and assessed for editing in buffers including protamine with and without glycerol. Buffers having different glycerol concentrations (1%, 5%, or 10%) and protamine concentrations (between 0-1.25 M) were assessed. A guide RNA targeting CD47 was associated with the respective TAGE agents to form ribonucleoproteins, and the ribonucleoproteins were co-incubated with T cells to test for editing. After a 1 hour co-incubation, the cells were washed with cell medium to remove additives and RNPs. Editing was measured using a phenotypic readout measuring the loss of surface CD47 using flow cytometry. The percentage of T cells that were edited under each condition are shown in FIG. 12 (top panel). Additionally, the levels of aggregation (turbidity) as measured by 340 nm light scattering are shown in FIG. 12 (bottom panel).

FIG. 13 graphically depicts the results of an ex vivo editing assay in which TAGE agents including 4×NLS-Cas9-2×NLS or AsCas12a were co-incubated with human T cells and assessed for editing in buffers including poly-glutamic acid (PGA) of different sizes (1500-5500 Da or 15,000 Da). A guide RNA targeting CD47 was associated with the respective TAGE agents to form ribonucleoproteins, and the ribonucleoproteins were co-incubated with T cells to test for editing. After a 1 hour co-incubation, the cells were washed with cell medium to remove additives and RNPs. Editing was measured using a phenotypic readout measuring the loss of surface CD47 using flow cytometry. The percentage of T cells that were edited under each condition are shown in FIG. 13.

FIG. 14 graphically depicts the results of an editing assay in which TAGE agents were co-incubated with primary human T cells at 0.7 nM, 7 nM or 70 nM TAGE in the presence of 0.5 M sucrose, and assessed for editing. The TAGE agents included TAGE26 (Cas9-2×NLS-SpyCatcher-4×NLS) with no antibody conjugated (“Unconjugated TAGE26”), TAGE26 including any one of three non-targeting (NT) (not T cell specific) antibodies (AB1, AB27 or AB21) (“AB1(NT)-TAGE26”, (“AB27(NT)-TAGE26”, and (“AB21(NT)-TAGE26”), which do not target an antigen expressed on the surface of human T cells), and TAGE26 conjugated with either of two targeting antibody (AB2, AB5) (“AB2-TAGE26” and (“AB5-TAGE26”), which each target an antigen expressed on the surface of human T cells. After a 1 hour co-incubation, the cells were washed with cell medium to remove additives and RNPs. Editing was measured using a phenotypic readout measuring the loss of surface CD47 using flow cytometry. The percentages of edited (CD47-) T cells from two donors edited under identical conditions were shown for each concentration of TAGE in the media.

FIGS. 15A-15E graphically depict the results of an editing assay in which TAGE agents were co-incubated with primary human T cells in the presence of different sugar alcohols including erythritol, glycerol, sucrose and xylitol. The TAGE agents included TAGE26 conjugated with antibody AB1 (non-targeting), TAGE26 conjugated with antibody AB2 (targeting), TAGE26 conjugated with antibody AB16 (targeting), TAGE26 conjugated with antibody AB17 (targeting) and TAGE26 conjugated with antibody AB21 (non-targeting). After a 1 hour co-incubation, the cells were washed with cell medium to remove additives and RNPs. The percentages of edited (CD47-) T cells under condition with AB1-TAGE26 (FIG. 15A), AB21-TAGE26 (FIG. 15B), AB2-TAGE26 (FIG. 15C), AB16-TAGE26 (FIG. 15D), or AB17-TAGE26 (FIG. 15E) were shown.

FIG. 16 graphically depicts the results of an editing assay in which TAGE agents were co-incubated with primary human T cells in the presence of sucrose or glycerol additive. The TAGE agents included TAGE26 conjugated with antibody AB1 (non-targeting), TAGE26 conjugated with antibody AB2 (targeting), TAGE26 conjugated with antibody AB16 (targeting), TAGE26 conjugated with antibody AB17 (targeting), and TAGE26 conjugated with AB21 (non-targeting). The percentages of edited (CD47-) After a 1 hour co-incubation, the cells were washed with cell medium to remove additives and RNPs. T cells under different conditions were shown.

FIGS. 17A and 17B graphically depict the results of an editing assay in which TAGE agents were co-incubated with primary human T cells in the presence of sucrose or salt additive. Antibodies were conjugated to Cas9 using the SpyTag/SpyCatcher system. A guide RNA targeting CD47 was associated with the respective TAGE agents to form ribonucleoproteins. RNPs at the indicated concentration were co-incubated with primary human T cells for 1 hour in T cell media with no additive (Baseline), 350 mM NaCl, 0.5 M sucrose, or 350 mM NaCl with 0.5 M sucrose. The cells were washed after one hour to remove additives and RNPs. The percentage of cells that were edited under each condition was measured using a phenotypic readout detecting the loss of surface CD47 by flow cytometry. Each data point represents a biological replicate. For each group, the horizontal line represents the mean and the error bars represent the standard deviation of the mean. The percentages of T cells that were edited under the indicated conditions with 700 nM TAGE (FIG. 17A) or 70 nM TAGE (FIG. 17B) were shown.

FIGS. 18A-18C graphically depict the results of an editing assay in which TAGE agents were co-incubated with primary human T cells under conditions where the transporter NHE1 was inhibited by amiloride derivatives. Antibodies were conjugated to Cas9 using the SpyTag/SpyCatcher system. A guide RNA targeting CD47 was associated with the respective TAGE agents to form ribonucleoproteins. Primary human T cells were pre-incubated with the indicated drug for 30′. Then, RNPs were added to cells at the indicated concentration and co-incubated for an additional hour. The cells were washed after one hour to remove inhibitors and RNPs. The percentage of cells that were edited under each condition was measured using a phenotypic readout detecting the loss of surface CD47 by flow cytometry. Each data point represents a biological replicate. For each group, the horizontal line represents the mean and the error bars represent the standard deviation of the mean. The percentages of T cells that were edited under condition with 3 μM TAGE26 (FIG. 18A), 0.35 μM AB27-TAGE26 (non-targeting) (FIG. 18B), or 0.35 μM AB2-TAGE26 (FIG. 18C) were shown. With respect to the descriptions in FIG. 18, NT guide is a TAGE with non-targeting guide RNA; MeOH is methanol (a control for DMA and EIPA; used at 0.5% v/v; DMSO is dimethyl sulfoxide, a control for Cyto D and Lat A (used at 1% v/v); DMA is 5-(N,N-dimethyl)-amiloride, an inhibitor of NHE1 (used at 100 μM); EIPA is 5-(N-ethyl-N-isopropyl)-amiloride, an inhibitor of NHE1, (used at 100 μM); Cyto D is cytochalasin D, an inhibitor of F-actin polymerization (used at 20 μM); and Lat A is latrunculin A, an inhibitor of F-actin polymerization (used at 10 μM).

FIG. 19 graphically depicts the results of an editing assay in which TAGE agents were co-incubated with primary human T cells under conditions with nystatin and dynasore treatment. Dynasore is an inhibitor of dynamin, which was used at 80 μM. Nystatin is an inhibitor of the lipid raft-caveolae endocytosis pathway, which was used at 108 μM. DMSO is dimethylsulfoxide, a control for nystatin and Dynasore, which was used at 2% v/v. Antibodies were conjugated to Cas9 using the SpyTag/SpyCatcher system. A guide RNA targeting CD47 was associated with the respective TAGE agents to form ribonucleoproteins. Primary human T cells were pre-incubated with the indicated drug for 30′. Then, RNPs were added to cells at the indicated concentration and co-incubated for an additional hour. The cells were washed after one hour to remove inhibitors and RNPs. The percentage of cells that were edited under each condition was measured using a phenotypic readout detecting the loss of surface CD47 by flow cytometry. Each data point represents a biological replicate. For each group, the horizontal line represents the mean and the error bars represent the standard deviation of the mean. The percentages of T cells that were edited under different conditions were shown.

FIG. 20 graphically depicts the results of an editing assay in which CPP-TAGE was incubated with primary human T cells at the presence of sucrose. A guide RNA targeting CD47 was associated with the CPP-TAGE Cas9-2×NLS-SpyCatcher-4×NLS to form ribonucleoprotein (RNP). RNP at the indicated concentration was co-incubated with primary human T cells for 1 hour in T cell media with no additive (Baseline) or in T cell media with 0.5 M sucrose. The cells were washed after one hour to remove additives and RNPs. The percentage of cells that were edited under each condition was measured using a phenotypic readout detecting the loss of surface CD47 by flow cytometry. Each data point represents a biological replicate. For each group, the horizontal line represents the mean and the error bars represent the standard deviation of the mean. The number above each group represents the mean of the group. The percentages of T cells that were edited under different conditions were shown.

FIG. 21 is a schematic of a nuclease antibody-binding agent described herein complexed with an antibody, antigen-binding agent, or antibody-like molecule to form a targeted active gene editing (TAGE) agent. In FIG. 1, the term “nuclease antibody-binding agent” refers to a site-directed modifying polypeptide including a nuclease.

### DETAILED DESCRIPTION OF THE INVENTION

### I. Definitions

The term “targeted active gene editing” or “TAGE” agent refers to a complex of molecules including a cell targeting agent (such as, but not limited to, an antigen binding polypeptide (e.g., an antibody or an antigen-binding portion thereof), a ligand, a cell penetrating peptide (CPP), or combinations thereof), that specifically binds to an extracellular target molecule (e.g., an extracellular protein or glycan, such as an extracellular protein on the cell surface) displayed on a cell membrane or otherwise promotes cellular internalization, and a site-directed modifying polypeptide (such as, but not limited to, an endonuclease) that recognizes a nucleic acid sequence. The cell targeting agent of a TAGE agent is associated with the site-directed modifying polypeptide such that at least the site-directed modifying polypeptide is internalized by a target cell, e.g., a cell expressing an extracellular molecule bound by the cell targeting agent. An example of a TAGE agent is an active CRISPR targeting or TAGE agent where the site directed polypeptide is a nucleic acid-guided DNA endonuclease (e.g., RNA-guided endonuclease or DNA-guided endonuclease), such as Cas9 or Cas12. In some embodiments, the TAGE agent includes at least one NLS. Notably, a TAGE agent can target any nucleic acid within a cell, including, but not limited to, a gene. TAGE agents are further described, for example in International Publication Numbers WO 2020/198151 and WO 2020/198160, as well as U.S. application Ser. Nos. 17/480,913 and 17/481,056, which are each hereby incorporated by reference in their entirety.

As used herein, a “site-directed modifying polypeptide” refers to a protein that is targeted to a specific nucleic acid sequence or set of similar sequences of a polynucleotide chain via recognition of the particular sequence(s) by the modifying polypeptide itself or an associated molecule (e.g., RNA), wherein the polypeptide can modify the polynucleotide chain. An example of a site-directed modifying polypeptide is a nucleic acid-guided nuclease, e.g., an RNA-guided nuclease.

As used herein, a “nucleic acid-guided nuclease” refers to a protein that is targeted to a specific nucleic acid sequence or set of similar sequences of a polynucleotide chain via recognition of the particular sequence(s) by the modifying polypeptide itself or an associated molecule (e.g., RNA), wherein the polypeptide can modify the polynucleotide chain. An example of a nucleic acid-guided nuclease is a RNA-guided endonuclease, such as Cas9.

The term “cell targeting agent” (alternatively referred to as an “extracellular cell membrane binding moiety”) refers to a protein (e.g., a ligand, a cell penetrating peptide, or an antigen binding agent) that, when conjugated with a conformation-specific NP binding agent that stably associates with a nucleoprotein comprising a nucleic acid-guided nuclease and a guide nucleic acid, enables at least the nucleoprotein to be targeted to the surface of a target cell or internalized by a target cell, i.e., a cell targeted by the cell targeting agent. In some embodiments, the cell targeting agent may be one that specifically binds to an extracellular target molecule (e.g., an extracellular protein, lipid, or glycan) displayed on a cell membrane. In such instances, the cell targeting agent can be associated with a nucleic acid-guided nuclease such that at least the nucleoprotein is internalized by a target cell, i.e., a cell expressing an extracellular molecule bound by the cell targeting agent.

As used herein, the term “modifying a nucleic acid” refers to any modification (i.e., change) to a nucleic acid targeted by a site-directed modifying polypeptide. Examples of such modifications include any changes to the amino acid sequence including, but not limited to, any insertion, deletion, or substitution of an amino acid residue in the nucleic acid sequence relative to a reference sequence (e.g., a wild-type or a native sequence). Such amino acid changes may, for example, may lead to a change in expression of a gene (e.g., an increase or decrease in expression) or replacement of a nucleic acid sequence. Modifications of nucleic acids can further include double stranded cleavage, single stranded cleavage, or binding of any RNA-guided endonuclease disclosed herein to a target site. Binding of a RNA-guided endonuclease can inhibit expression of the nucleic acid or can increase expression of any nucleic acid in operable linkage to the nucleic acid comprising the target site.

The terms “polypeptide” or “protein”, as used interchangeably herein, refer to any polymeric chain of amino acids. The term “polypeptide” encompasses native or artificial proteins, protein fragments and polypeptide analogs of a protein sequence.

The term “conjugation moiety” as used herein refers to a moiety that is capable of conjugating two more or more molecules, such as an antigen binding protein, a CPP, or a ligand and a site-directed modifying polypeptide. The term “conjugation,” as used herein, refers to the physical or chemical complexation formed between a molecule (for e.g. an antigen binding protein (e.g., an antibody), CPP, or ligand) and the second molecule (e.g. a site-directed modifying polypeptide, therapeutic agent, drug or a targeting molecule). The chemical complexation constitutes specifically a bond or chemical moiety formed between a functional group of a first molecule (e.g., an antigen binding protein (e.g., an antibody), CPP, or ligand) with a functional group of a second molecule (e.g., a site-directed modifying polypeptide, a therapeutic agent or drug). Such bonds include, but are not limited to, covalent linkages and non-covalent bonds, while such chemical moieties include, but are not limited to, esters, carbonates, imines phosphate esters, hydrazones, acetals, orthoesters, peptide linkages, and oligonucleotide linkages. In one embodiment, conjugation is achieved via a physical association or non-covalent complexation.

As used herein, the term “ligand” refers to a molecule that is capable of specifically binding to another molecule on or in a cell, such as one or more cell surface receptors, and includes molecules such as proteins, hormones, neurotransmitters, cytokines, growth factors, cell adhesion molecules, or nutrients. Generally, a ligand that binds to another specific molecule or molecules. For example, a ligand may bind to a receptor. A site-specific modifying polypeptide (e.g., nuclease) of TAGE agent can be associated with one or more ligands through covalent or non-covalent linkage. Examples of ligands useful herein, or targets bound by ligands, and further description of ligands in general, are disclosed in Bryant & Stow (2005). Traffic, 6(10), 947-953; Olsnes et al. (2003). Physiological reviews, 83(1), 163-182; and Planque, N. (2006). Cell Communication and Signaling, 4(1), 7, which are incorporated herein by reference.

As used herein, the term “target cell” refers to a cell or population of cells, such as mammalian cells (e.g., human cells), which includes a nucleic acid sequence in which site-directed modification of the nucleic acid is desired (e.g., to produce a genetically-modified cell in vivo or ex vivo). In some instances, a target cell displays on its cell membrane an extracellular molecule (e.g., an extracellular protein such as a receptor or a ligand, or glycan) specifically bound by an extracellular cell membrane binding moiety of the TAGE agent.

As used herein, the term “genetically-modified cell” refers to a cell, or an ancestor thereof, in which a DNA sequence has been deliberately modified by a site-directed modifying polypeptide.

As used herein, the term “nucleic acid” refers to a molecule comprising nucleotides, including a polynucleotide, an oligonucleotide, or other DNA or RNA. In one embodiment, a nucleic acid is present in a cell and can be transmitted to progeny of the cell via cell division. In some instances, a nucleic acid is a gene (e.g., an endogenous gene) found within the genome of a cell within its chromosomes. In other instances, a nucleic acid is a mammalian expression vector that has been transfected into a cell. DNA that is incorporated into the genome of a cell using, e.g., transfection methods, is also considered within the scope of a “nucleic acid” as used herein, even if the incorporated DNA is not meant to be transmitted to progeny cells.

As used herein, the term “endosomal escape agent” or “endosomal release agent” refers to an agent (e.g., a peptide) that, when conjugated to a molecule (e.g., a polypeptide, such as a site-directed modifying polypeptide), is capable of promoting release of the molecule from an endosome within a cell. Polypeptides that remain within endosomes can eventually be targeted for degradation or recycling rather than released into the cytoplasm or trafficked to a desired subcellular destination. Accordingly, in some embodiments, a TAGE agent comprises an endosomal escape agent.

As used herein, the term “stably associated” when used in the context of a TAGE agent, refers to the ability of the cell targeting agent and the site-directed modifying polypeptide to complex in such a way that the complex can be internalized into a target cell such that nucleic acid editing can occur within the cell. Examples of ways to determine if a TAGE agent is stably associated include in vitro assays whereby association of the complex is determined following exposure of a cell to the TAGE agent, e.g., by determining whether gene editing occurred using a standard gene editing systems. Examples of such assays are known in the art, such as SDS-PAGE, Western blot, size exclusion chromatography (SEC), and electrophoretic mobility shift assay to determine protein complexes; PCR amplification, direct sequencing (e.g., next-generation sequencing or Sanger sequencing), enzymatic cleavage of a locus with a nuclease (e.g., Celery) of the gene locus to confirm editing; and indirect phenotypic assays that measure the downstream effects of editing a specific gene, such as loss of a protein as measured by Western blot or flow cytometry or generation of a functional protein, as measured by functional assays.

The term “cell-penetrating peptide” (CPP) refers to a peptide, generally of about 5-60 amino acid residues (e.g., 5-10, 10-15, 15-20, 20-25, 25-30, 30-35, 35-40, 40-45, 45-50, 50-55, or 55-60 amino acid resides) in length, that can facilitate cellular uptake of a conjugated molecule, particularly one or more site-specific modifying polypeptides. A CPP can also be characterized in certain embodiments as being able to facilitate the movement or traversal of a molecular conjugate across/through one or more of a lipid bilayer, micelle, cell membrane, organelle membrane (e.g., nuclear membrane), vesicle membrane, or cell wall. A CPP herein can be cationic, amphipathic, or hydrophobic in certain embodiments. Examples of CPPs useful herein, and further description of CPPs in general, are disclosed in Borrelli, Antonella, et al. Molecules 23.2 (2018): 295; Milletti, Francesca. Drug discovery today 17.15-16 (2012): 850-860, which are incorporated herein by reference. Further, there exists a database of experimentally validated CPPs (CPPsite, Gautam et al., 2012). The CPP of a TAGE agent of the invention can be any known CPP, such as a CPP shown in the CPP site database.

As used herein, the term “nuclear localization signal” or “NLS” refers to a peptide that, when conjugated to a molecule (e.g., a polypeptide, such as a site-directed modifying polypeptide), is capable of promoting import of the molecule into the cell nucleus by nuclear transport. The NLS can, for example, direct transport of a protein with which it is associated from the cytoplasm of a cell across the nuclear envelope barrier. The NLS is intended to encompass not only the nuclear localization sequence of a particular peptide, but also derivatives thereof that are capable of directing translocation of a cytoplasmic polypeptide across the nuclear envelope barrier. In some embodiments, one or more NLSs (e.g., 1, 2, 3, 4, 5, 6, 7, 8, 2-6, 3-7, 4-8, 5-9, 6-10, 7-10, 8-10 NLSs) can be attached to the N-terminus, the C-terminus, or both the N- and C-termini of the polypeptide of a TAGE agent herein.

As used herein, the term “specifically binds” in the context of an antigen binding polypeptide refers to an antigen-binding polypeptide which recognizes and binds to an antigen present in a sample, but which antigen binding polypeptide does not substantially recognize or bind other molecules in the sample. In one embodiment, an antigen binding polypeptide that specifically binds to an antigen, binds to an antigen with an Kd of at least about 1×10⁻⁴, 1×10⁻⁵, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more as determined by surface plasmon resonance or other approaches known in the art (e.g., filter binding assay, fluorescence polarization, isothermal titration calorimetry), including those described further herein. In one embodiment, an antigen binding polypeptide specifically binds to an antigen if the antigen binding polypeptide binds to an antigen with an affinity that is at least two-fold greater as determined by surface plasmon resonance than its affinity for a nonspecific antigen. When used in the context of a ligand, the term “specifically binds” refers to the ability of a ligand to recognize and bind to its respective receptor(s). When used in the context of a CPP, the term “specifically binds” refers to the ability of CPPs to translocate a cell's membrane. In some instances, when a CPP(s) and either an antibody or a ligand are combined as a TAGE agent, the TAGE agent may display the specific binding properties of both the antibody or ligand and the CPP(s). For example, in such instances, the antibody or ligand of the TAGE agent may confer specific binding to an extracellular cell surface molecule, such as a cell surface protein, while the CPP(s) confers enhanced ability of the TAGE agent to translocate across a cell membrane.

The term “antigen binding polypeptide” as used herein refers to a protein that binds to a specified target antigen, such as an extracellular cell membrane-bound protein (e.g., a cell surface protein). Examples of an antigen binding polypeptide include an antibody, antigen-binding fragment of an antibody, and an antibody mimetic. In certain embodiments, an antigen-binding polypeptide is an antigen binding peptide.

The term “antibody” is used herein in the broadest sense and encompasses various antibody structures, including but not limited to monoclonal antibodies, polyclonal antibodies, multispecific antibodies (e.g., bispecific antibodies), nanobodies, monobodies, and antibody fragments so long as they exhibit the desired antigen-binding activity.

The term “antibody” includes an immunoglobulin molecule comprising four polypeptide chains, two heavy (H) chains and two light (L) chains inter-connected by disulfide bonds, as well as multimers thereof (e.g., IgM). Each heavy chain (HC) comprises a heavy chain variable region (or domain) (abbreviated herein as HCVR or VH) and a heavy chain constant region (or domain). The heavy chain constant region comprises three domains, CH1, CH2 and CH3. Each light chain (LC) comprises a light chain variable region (abbreviated herein as LCVR or VL) and a light chain constant region. The light chain constant region comprises one domain (CL1). Each VH and VL is composed of three complementarity determining regions (CDRs) and four framework (FRs), arranged from amino-terminus to carboxy-terminus in the following order: FR1, CDR1, FR2, CDR2, 1-R3, CDR3, FR4 Immunoglobulin molecules can be of any type (e.g., IgG, IgE, IgM, IgD, IgA and IgY), class (e.g., IgG1, IgG2, IgG3, IgG4, IgA1 and IgA2) or subclass. Thus, the VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “cell medium” as used herein, refers to a buffered solution which is suitable for cells to be stored or incubated for any given period of time. Cell medium refers to a buffered solution in which a stable environment is provided for cells to be contacted with a site directed modifying polypeptide.

The term “aqueous formulation”, as used herein, refers to a liquid solution in which water is the dissolving medium or solvent.

The term “stable” as used herein in the context of a formulation, is intended to mean that the composition substantially retains its physical stability, and/or conformational stability and/or colloidal stability upon storage. The composition may also retain chemical stability and/or biological activity of, for example, a TAGE molecule therein. Stability can be assessed, in certain embodiments, according to a characteristic(s) of an active molecule therein, e.g., resistance of a ribonucleoprotein (RNP) in the formulation to aggregation, degradation or fragmentation under certain conditions. In one embodiment, stability of a formulation refers to the ability of the formulation to maintain the chemical and physical stability of a site-directed modifying polypeptide or a TAGE agent therein.

As used herein, “biological activity” of an RNP refers to the ability of the RNP to cleave DNA in vitro and/or edit a target DNA sequence ex vivo or in vivo.

As used herein, the term “refolded guide RNA” refers to a guide RNA that has been heated (e.g., to a temperature that at least partially denatures the gRNA, e.g., at least 70° C.) and then subsequently cooled (e.g., to a temperature that allows the gRNA to renature, e.g., 20 to 25° C., or 2-8° C.) such that it refolds to its secondary structure.

As used herein, the term “sugar alcohol” or “polyol” refers to molecules having the general formula HOCH2(CHOH)nCH2OH. Examples of sugar alcohols include, but are not limited to, glycerol, erythritol, xylitol, sorbitol, mannitol, and inositol.

The term “sugar” as used herein denotes a monosaccharide or an oligosaccharide. A monosaccharide is a monomeric carbohydrate which is not hydrolysable by acids, including simple sugars and their derivatives, e.g. aminosugars. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra- penta- and so forth saccharide. In contrast to polysaccharides, the monosaccharides and oligosaccharides are water soluble. Examples of oligosaccharides include sucrose, trehalose, lactose, maltose and raffinose. In particular, a sugar is sucrose.

Additional definitions are described in the sections below.

Various aspects of the invention are described in further detail in the following subsections.

### II. Stable Aqueous TAGE Agent Formulations

Buffer conditions for ribonucleoprotein complexes (RNPs) are an important consideration for storage stability and activity of RNPs. RNPs can be prone to aggregation, especially in standard buffers (e.g., Phosphate Buffered Saline (PBS)) and in physiological conditions (neutral pH and low salt concentrations), which limits the enzymatic activity of the RNPs and may alter their cell penetrating properties. RNP aggregation can also occur when RNPs are formed using gRNAs that have not been re-folded, in accordance with the methods herein. Moreover, salt concentration and overall solution tonicity are important factors that affect cells' ability to uptake proteins (D.S. D'Astolfo, et al., Efficient intracellular delivery of native proteins. Cell 161, 674-690 (2015)). Therefore, successful production of RNPs requires buffer conditions that maintain RNPs in a stable, unaggregated state during extended storage such that their biological activity is preserved.

Accordingly, provided here in are stable aqueous formulations for RNPs, such as Targeted Active Gene Editing (TAGE) agents. The stable aqueous formulations provided can maintain the physical, chemical, and/or biological stability of the RNPs upon storage. The storage period is generally selected based on the intended shelf-life of the formulation. Various analytical techniques for measuring protein stability are available in the art and are reviewed, for example, in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993).

### Stability

In one embodiment, the stable aqueous formulation substantially retains the physical, chemical stability, and/or biological activity of an RNP (e.g., TAGE agent). In certain embodiments, the stable aqueous formulation substantially retains the physical stability of an RNP (e.g., TAGE agent). A protein retains its physical stability in a pharmaceutical formulation if it shows no signs or very little of aggregation, precipitation and/or denaturation upon visual examination of color and/or clarity, or as measured by UV/Vis light scattering or by size exclusion chromatography, for example.

In certain embodiments, the stable aqueous formulation substantially retains the chemical stability of an RNP (e.g., TAGE agent). Chemical stability can be assessed by detecting and quantifying chemically altered forms of the protein and RNA components. Chemical alteration may involve size modification (e.g. clipping) which can be evaluated using chromatography (e.g., size exclusion chromatography (SEC), reverse-phase chromatography, and ion exchange chromatography), denaturing PAGE (SDS-PAGE for protein, Urea/PAGE for RNA) and/or mass spectrometry (MS), for example. Other types of chemical alteration include charge alteration (e.g. occurring as a result of deamidation) which can be evaluated by ion-exchange chromatography or icIEF, for example. A protein, RNA or RNP retains its chemical stability in a formulation, if the chemical stability at a given time is such that the protein, RNA or RNP is considered to still retain its biological activity as defined below.

In certain embodiments, the stable aqueous formulation substantially retains the biological stability of an RNP (e.g., TAGE agent). Biological activity of an RNP refers to its ability to cleave or edit DNA in vitro or ex vivo (e.g., in vitro DNA cleavage activity, ex vivo editing activity, and/or in vivo editing activity). In some embodiments, the stable aqueous formulation retains the physical stability, the chemical stability, and the biological stability of the RNP (e.g., TAGE agent). Editing activity can be tested using methods described herein, as well as those known in the art.

In one aspect, an RNP (e.g., the TAGE agent) of the aqueous formulation disclosed herein is stable upon storage. Aqueous formulations can be stored, for example, at room temperature (e.g., 20 to 25° C.) refrigerated (e.g., 2-8° C.), or frozen (e.g., −20° C. to −70° C.) for storage. For example, in some embodiments, the aqueous formulation is stable upon storage at about 15° C., 16° C., 18° C., 20° C., 22° C., 24° C., or 25° C. (e.g., for at least about 4 weeks, at least about 2 months, at least about 3 months, or at least about 6 months, or at least about 9 months, or at least about 12 months). in some embodiments, the aqueous formulation is stable upon storage at about 2° C., 4° C., 5° C., 6° C., 8° C., 10° C., 12° C., 14° C., or 15° C. (e.g., for at least about 3 months, at least about 1 year, at least about 2 years, at least about 3 years or longer). In some embodiments, the aqueous formulation is stable upon storage at −15° C., −20° C., −25° C., −30° C., −35° C., −40° C., −45° C., −50° C., −55° C., −60° C., −65° C., −70° C., −75° C., −80° C., or −85° C. (e.g., for at least about 4 weeks; at least about 3 months, at least about 6 months, at least about 9 months, at least about 1 year, at least about 2 years, at least about 3 years, at least about 4 years or longer).

Stability of an RNP (e.g., a TAGE agent) can be measured at a selected temperature for a selected time period. For example, in some embodiments, the aqueous formulation is stable at about 5° C. to about 30° C. (e.g., 5-10° C., 10-15° C., 15-20° C., 20-25° C., 25-30° C., 5-30° C., 10-25° C., 15-25° C.) for at least about 1 month, at least about 3 months, at least about 6 months, at least about 9 months, at least about 12 months, at least about 18 months, at least about 24 months, at least about 30 months, or at least about 36 months; and/or stable at about −20° C. to about −80° C. for at least about 1 month, at least about 3 months, at least about 6 months, at least about 9 months, at least about 12 months, at least about 18 months, at least about 24 months, at least about 30 months, at least about 36 months, at least about 42 months, or at least about 48 months.

In some embodiments, an RNP (e.g., TAGE agent) of the stable aqueous formulation is stable upon storage at about 25° C. for at least about 4 weeks, at least about 2 months, at least about 3 months, or at least about 6 months, or at least about 9 months, or at least about 12 months. In some embodiments, the RNP (e.g., TAGE agent) of the stable aqueous formulation is stable upon storage at about 22° C. for at least about 4 weeks, at least about 2 months, at least about 3 months, or at least about 6 months, or at least about 9 months, or at least about 12 months. Alternatively or in addition, the RNP in the formulation may be stable upon storage at about 15° C. for at least about 4 weeks, at least about 3 months, at least about 6 months, at least about 9 months, at least about 1 year, or longer. In some embodiments, the RNP of the stable aqueous formulation is stable upon storage at about 2-8° C. for at least about 3 months, at least about 1 year, at least about 2 years, at least about 3 years or longer. Alternatively or in addition, the RNP in the formulation may be stable upon storage at freezing (e.g., about −20° C. to −80° C.) for at least about 4 weeks; at least about 3 months, at least about 6 months, at least about 9 months, at least about 1 year, at least about 2 years, at least about 3 years, at least about 4 years or longer. In such embodiments, stability is assessed by a real-time stability assay. Various stability assays are available to the skilled practitioner for confirming the stability of the formulation, such as those described further herein.

In some embodiments, an RNP (e.g., a TAGE agent) of the stable aqueous formulation are stable under accelerated stability conditions. In accelerated stability testing, the RNP is stressed at higher temperatures (i.e., warmer than ambient) and the amount of heat input to cause product failure or degradation is determined. This information is then used to project shelf life or to compare the relative stability of formulations. In addition to temperature, stress conditions applied to the formulation during accelerated stability testing can include moisture, light, agitation, gravity, pH, and packing conditions. Standard assays and conditions can be used to assess accelerated stability of an agent, as described, for example, in Bajaj, et al. J App Pharm Sci, 2(3), 2012: 129-138, which is hereby incorporated in its entirety by reference. In some such instances, accelerated stability of the aqueous formulation is assessed at 40° C. For example, the aqueous formulation may be stable at about 40° C. for at least about 3 days, 5 days, 1 week, 2 weeks, 3 weeks, 4 weeks, 5 weeks or 6 weeks. In another embodiment, the formulation is stable at about 40° C. for at least about 2-4 weeks, at least about 3 months, at least about 6 months, at least about 9 months, at least about 12 months, or at least about 18 months.

In some cases, RNP formulations are frozen for storage. Accordingly, it is desirable that the formulation be relatively stable under such conditions, including under freeze/thaw cycles. As used herein, the term a “freeze/thaw cycle” refers to freezing the formulation at a temperature below 0° C. (e.g., at about −10° C., −20° C., −30° C., −40° C., −50° C., −60° C., −70° C., −80° C., or −85° C.) followed by thawing the formulation at a temperature above 0° C. (e.g., at about 2° C., 4° C., 6° C., 8° C., 10° C., 120 C, 14° C., 16° C., 18° C., 20° C., 22° C., 24° C., or 250 C). One method of determining the suitability of a formulation is to subject a sample formulation to at least two, e.g., three to ten cycles of freezing and thawing (for example by fast thaw at room temperature or slow thaw on ice), determining the amount of low molecular weight (LMVV) species and/or high molecular weight (HMVV) species that accumulate after the freeze-thaw cycles and comparing it to the amount of LMW species or HMW species present in the sample prior to the freeze-thaw procedure. An increase in the LMW or HMW species indicates decreased stability of a protein stored as part of the formulation. Size exclusion high performance liquid chromatography (SEC-HPLC) can be used to determine the presence of LMW and HMW species.

Accordingly, in some embodiments, the RNP (e.g., TAGE agent) of the stable aqueous formulation is stable following freezing and thawing, for example following 1, 2, 3, 4, 5, 6, 7, 8, 9, 10, or more than 10 cycles of freezing and thawing. In one embodiment, the RNP (e.g., TAGE agent) of the formulation remains stable after being subjected to one freeze/thaw cycle. In another embodiment, the RNP (e.g., TAGE agent) of the formulation remains stable after being subjected to two freeze/thaw cycles. In yet another embodiment, the RNP (e.g., TAGE agent) of the formulation remains stable after being subjected to three freeze/thaw cycles. In a further embodiment, the RNP (e.g., TAGE agent) of the formulation remains stable after being subjected to four freeze/thaw cycles. In another embodiment, the RNP (e.g., TAGE agent) of the formulation remains stable after being subjected to five freeze/thaw cycles.

The stability of a liquid formulation can be evaluated qualitatively and/or quantitatively in a variety of different ways (see, e.g., Analytical Techniques for Biopharmaceutical Development, Rodriguez-Diaz et al. eds. Informa Healthcare (2005)), including evaluation of dimer, multimer and/or aggregate formation (for example using size exclusion chromatography (SEC), native electrospray ionization mass spectrometry, negative stain electron microscopy, analytical ultracentrifugation, light scattering (photon correlation spectroscopy, dynamic light scattering (DLS), static light scattering, multi-angle laser light scattering (MALS)), flow-based microscopic imaging, electronic impedance (coulter) counting, light obscuration or other liquid particle counting system, by measuring turbidity, by density gradient centrifugation, and/or by visual inspection); by assessing charge heterogeneity using cation exchange chromatography (CIEX; see also Vlasak and Ionescu, Curr. Pharm. Biotechnol. 9:468-481 (2008) and Harris et al. J. Chromatogr. B Biomed. Sci. Appl. 752:233-245 (2001)), isoelectric focusing (IEF), e.g. capillary technique (cIEF), or capillary zone electrophoresis; amino-terminal or carboxy-terminal sequence analysis; mass spectrometric analysis; PAGE, SEC, or reverse-phase analysis to compare fragmented, intact and multimeric (i.e., dimeric, trimeric, etc.) RNPs; peptide map (for example tryptic or LYS-C) analysis; evaluating biological activity, DNA binding, DNA cleavage, or DNA editing of the TAGE agent, and the like.

Lack of stability or instability may involve, for example, aggregation (e.g., non-covalent soluble aggregation, covalent soluble aggregation (e.g., disulfide bond rearrangement/scrambling), insoluble aggregation), deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, and the like. Biological activity or DNA editing or cleavage function, e.g., binding of the RNP to DNA, in vitro DNA cleavage, ex vivo DNA editing, or in vivo DNA editing, can be evaluated using various techniques available to the skilled practitioner.

Monomeric RNP content and/or aggregate RNP content (e.g., as dimers, trimers, tetramers, pentamers, oligomers and higher-order aggregates), i.e., in the liquid formulation can be measured by SEC, analytical ultracentrifugation, light scattering (DLS or MALS), native electrospray ionization mass spectrometry, negative stain electron microscopy, or nanoscale measurement, such as nanoparticle tracking analysis NTA, NanoSight Ltd, Wiltshire, UK). Resolution, characterization and quantification of aggregate can be achieved in a number of ways, including increasing the length of the SEC column separation, e.g., by a longer column or by serial attachment of a second or more SEC column(s) in line with the initial analytical SEC column, supplementing SEC quantification of monomers with light scattering, or by using NTA. In certain embodiments, the stability of the RNP (e.g., TAGE agent) is measured by light scattering as detected by UV/Vis absorbance spectroscopy (e.g., at an absorbance of 340 nm and/or 600 nm).

In some embodiments, the stable aqueous formulations herein comprise a reduced level of aggregates of the RNP (e.g., the TAGE agent) relative to a reference level as detected by UV/Vis absorbance spectroscopy. In one embodiments, the reference level is the level of aggregates in a standard buffer, such as PBS (e.g., under the same storage conditions (temperature, time, humidity) as the conditions under which the stable aqueous formulation was stored prior to evaluation for aggregates). In another embodiment, the reference level is a pre-defined threshold level of aggregates (e.g., a pre-defined threshold for percent aggregates). In some embodiments, the percent aggregates of the RNP is determined by the relative area under of the curve corresponding to aggregates of TAGE agents that elute from a Size Exclusion Chromatography (SEC) resin. An RNP aggregate can be determined according to known methods in the art, including, but not limited to, SEC, analytical ultracentrifugation, light scattering (DLS or MALS), MS, nanoscale measurement, or UV/Vis absorbance spectroscopy.

In one embodiment, the stable aqueous formulation has at least 90% monomeric RNPs, least 85% monomeric RNPs, least 90% monomeric RNPs, at least 95% monomeric RNPs, or 97 to 99% monomeric RNPs. Monomeric RNPs can be determined by SEC analysis.

In another embodiment, the stable aqueous formulation has less than 20% RNP aggregates, has less than 15% RNP aggregates, has less than 10% RNP aggregates, less than 5% RNP aggregates, less than 2.5% RNP aggregates, less than 1.5% RNP aggregates, or less than 1.0% RNP aggregates s. In another aspect, the stable aqueous formulation comprises 96% RNP monomers and/or 2.5% RNP aggregates. In some embodiments, aggregates of the TAGE agent are not detectably present in the formulation as determined by UV/Vis absorbance spectroscopy.

In any of the embodiments herein, stability of the aqueous formulation can be determined relative to other standard buffers, such as PBS. In some embodiments, the RNP (e.g., TAGE agent) of the stable aqueous formulations provided herein have increased stability (e.g., as measured by any one of the methods provided herein) relative to the same RNP in PBS. For example, in one embodiment, the RNP (e.g., TAGE agent) has increased stability during storage at about 4° C., relative to the same RNP (e.g., TAGE agent) stored in PBS buffer for the same time period (e.g., after storage for at least about 4 weeks, at least about 3 months, at least about 6 months, at least about 9 months, at least about 1 year). In another embodiment, the RNP (e.g., TAGE agent) has increased stability during storage at about 22° C., relative to the same RNP (e.g., TAGE agent) stored in PBS buffer for the same time period (e.g., after storage for at least about 4 weeks, at least about 3 months, at least about 6 months, at least about 9 months, at least about 1 year). In one embodiment, the RNP (e.g., TAGE agent) has increased stability during storage at about −20° C. or colder, relative to the same RNP (e.g., TAGE agent) stored in PBS buffer for the same time period (e.g., after storage for at least about 4 weeks, at least about 3 months, at least about 6 months, at least about 9 months, at least about 1 year). In one embodiment, the RNP (e.g., TAGE agent) has increased stability during storage at about 40° C., relative to the same RNP (e.g., TAGE agent) stored in PBS buffer for the same time period (e.g., after storage for at least about 4 weeks, at least about 3 months, at least about 6 months, at least about 9 months, at least about 1 year). In yet another embodiment, the RNP (e.g., TAGE agent) has increased stability following one or more freeze/cycles (e.g., 1, 2, 3, 4, 5, or more than 5 freeze/thaw cycles), relative to the same RNP (e.g., TAGE agent) stored in PBS buffer subjected to the same number of freeze/thaw cycles.

### Aqueous Formulations

The aqueous formulations provided herein include a salt, sugar, and free amino acid at a concentration sufficient to stabilize an RNP, such as TAGE agent. Stability of the formulation can be assessed by any methods standard in the art, including those described above.

The RNP of the stable aqueous formulation can include a TAGE agent described herein (e.g., see Section V). A TAGE agent comprises a cell targeting agent and a site-directed modifying polypeptide that recognizes a nucleic acid. In some embodiments, the site-directed modifying polypeptide that recognizes a nucleic acid is a nucleic acid-guided nuclease, such as an RNA-guided nuclease. For example, in some embodiments, the RNA-guided nuclease is Class 2 Cas polypeptide, such as a Type II Cas polypeptide (e.g., Cas9) or a Type V Cas polypeptide (e.g., Cas12). In some embodiments, the formulation further comprises a guide nucleic acid (gNA), wherein the gNA and the nucleic acid-guided nuclease form a nucleoprotein. In some embodiments, the guide nucleic acid is a guide RNA (gRNA), the nucleic acid-guided nuclease is an RNA-guided nuclease, and the gRNA and RNA-guided nuclease form a ribonucleoprotein. The cell targeting agent of the TAGE agent can be, for example, a ligand, a cell penetrating peptide, or an antigen-binding polypeptide, including any of those set forth in Section III.

In one embodiment, the liquid formulation comprises at least about 0.1 μM, at least about 0.2 μM, at least about 0.5 μM, at least about 0.6 μM, at least about 0.8 μM, at least about 1 μM, at least about 1.2 μM, at least about 1.5 μM, at least about 1.6 μM, at least about 1.8 μM, or at least about 2 μM of the TAGE agent. In one embodiment, the liquid formulation comprises at least about 2 μM, at least about 4 μM, at least about 6 μM, at least about 8 μM, at least about 10 μM, at least about 12 μM, at least about 14 μM, at least about 16 μM, at least about 18 μM, or at least about 20 μM of the TAGE agent. In some embodiments, the liquid formulation comprises at least about 20 μM, at least about 25 μM, at least about 30 μM, at least about 35 μM, at least about 40 μM, at least about 45 μM, at least about 50 μM, at least about 55 μM, at least about 60 μM, at least about 65 μM, at least about 70 μM, at least about 75 μM, at least about 80 μM, at least about 85 μM, at least about 90 μM, at least about 95 μM, at least about 100 μM of the TAGE agent.

In some embodiments, the liquid formulation comprises 0.1-50 μM of the TAGE agent. In some embodiments, the liquid formulation comprises 0.1-1 μM, 1-10 μM, 5-15 μM, 10-20 μM, 15-25 μM, 20-30 μM, 25-35 μM, 30-40 μM, 35-45 μM, or 40-50 μM. In some embodiments, the liquid formulation comprises 50-100 μM of the TAGE agent. In some embodiments, the liquid formulation comprises 50-60 μM, 55-65 μM, 60-70 μM, 65-75 μM, 70-80 μM, 75-85 μM, 80-90 μM, 85-95 μM, or 90-100 μM. In some embodiments, the liquid formulation comprises 0.1-0.5 μM, 0.25-0.75 μM, 0.5-1 μM, 0.75-1.5 μM, 1-2 μM, 1.5-2.5 μM, 2-3 μM, 2.5-3.5 μM, 3-4 μM, 3.5-4.5 μM, 4-5 μM, 4.5-5.5 μM, 5-6 μM, 5.5-6.5 μM, 6-7 μM, 6.5-7.5 μM, 7-8 μM, 7.5-8.5 μM, 8-9 μM, 8.5-9.5 μM, 9-10 μM, 10-12 μM, 11-13 μM, 12-14 μM, 13-15 μM, 14-16 μM, 15-17 μM, 16-18 μM, 17-19 μM, 18-20 μM, 20-25 μM, 24-28 μM, 25-30 μM, 28-32 μM, 30-35 μM, 34-38 μM, 40-45 μM, 38-42 μM, 45-50 μM, 44-48 μM, 45-50 μM, 48-52 μM, 50-55 μM, 54-58 μM, 55-60 μM, 64-68 μM, 65-70 μM, 68-72 μM, 70-75 μM, 74-78 μM, 75-80 μM, 78-82 μM, 80-85 μM, 84-88 μM, 85-90 μM, 88-92 μM, 90-95 μM, 94-98 μM, or 95-100 μM of the TAGE agent.

In some embodiments, the formulation includes a gRNA (e.g., single guide RNA (sgRNA) or a cr:trRNA) complexed with the TAGE agent to form a ribonucleoprotein. In one embodiment, the gRNA is a refolded gRNA. As demonstrated in Examples 2 and 3, refolded gRNA can elute as a single peak from a SEC column, whereas gRNA that has not undergone this refolding process tends to elute as multiple peaks over a wider time span and can interfere with detection of RNPs. Accordingly, refolded gRNA can be characterized based in its capability to elute as a single peak from a Size Exclusion Chromatography resin when the gRNA is not complexed to the RNP. To refold gRNA, the gRNA is heated (e.g., to a temperature that at least partially denature the gRNA) and then subsequently cooled (e.g., to a temperature that allows the gRNA to renature). For example, in some embodiments, the gRNA can be refolded by heating the gRNA to a temperature of at least 70° C. and then cooling the gRNA at or below room temperature (e.g., 2° C.-25° C.).

Accordingly, in some embodiments, the gRNA of the aqueous formulation is one that has been pre-treated under conditions effective to refold the gRNA. In some such embodiments, the gRNA is refolded by heating the gRNA to a temperature of at least 60° C. (e.g., 60° C., 65° C., 70° C., 75° C., 80° C., or more) and cooling the gRNA at ambient temperature (e.g., 20° C., 21° C., 22° C., 23° C., 24° C., or 25° C.). In some embodiments, the gRNA is refolded by heating the gRNA to a temperature of at least 60° C. (e.g., 60° C., 65° C., 70° C., 75° C., 80° C., or more) and cooling the gRNA at a temperature of 10° C.-18° C. (e.g., 10° C., 12° C., 14° C., 16° C., or 18° C.). In some embodiments, the gRNA is refolded by heating the gRNA to a temperature of at least 60° C. (e.g., 60° C., 65° C., 70° C., 75° C., 80° C., or more) and cooling the gRNA at a temperature of 2° C.-8° C. (e.g., 2° C., 4° C., 6° C., or 8° C.). In certain embodiments, the gRNA is heated for at least 1 minute (e.g., 1 minute, 2 minutes, 3 minutes, 4 minutes, 5 minutes, 6 minutes, 7 minutes, 8 minutes, 10 minutes, or more than 10 minutes). In some embodiments, the gRNA is cooled for at least 1 minute (e.g., 1 minute, 2 minutes, 3 minutes, 4 minutes, 5 minutes, 6 minutes, 7 minutes, 8 minutes, 10 minutes, or more than 10 minutes). In one embodiment, the gRNA is refolded by heating the gRNA for at least 1 minute at 70° C. and cooling the gRNA at ambient temperature (e.g., 20° C.-25° C.) for at least 2 minutes. In one embodiment, the gRNA is refolded by heating the gRNA for at least 1 minute at 70° C. and cooling the gRNA at ambient temperature of at least 2 minutes. In one embodiment, the gRNA is refolded by heating the gRNA for at least 1 minute at 70° C. and cooling the gRNA at ambient temperature of at least 10 minutes.

The stable aqueous formulations herein additionally include a salt. For example, the formulation can include a monovalent salt, such as a monovalent sodium salt or a monovalent potassium salt. For example, in some embodiments the salt is NaCl, KCl, and potassium glutamate. Other examples include CaCl₂, MgCl₂, and potassium phosphate at effective concentrations where these salts do not denature the protein. In one embodiment, the salt is NaCl. In another embodiment, the salt is KCl. In some embodiments, the concentration of the salt in the formulation is at least about 50 mM, at least about 75 mM, at least about 100 mM, at least about 125 mM, at least about 150 mM, at least about 175 mM, at least about 200 mM, at least about 300 mM, at least about 400 mM, at least about 500 mM, at least about 600 mM, at least about 700 mM, at least about 800 mM, at least about 900 mM, at least about 1000 mM, at least about 1100 mM, at least about 1200 mM, at least about 1300 mM, at least about 1400 mM, at least about 1500 mM, at least about 1600 mM, at least about 1700 mM, at least about 1800 mM, or at least about 1900 mM of the salt. In a further embodiment, the formulation may comprise between about 50 and about 2000 mM, between about 50 mM and 1500 mM, between about 50 mM and about 1000 mM, between about 50 mM and about 750 mM, between about 50 mM and about 500 mM, between about 50 mM and about 250 mM, between about 50 mM and about 100 mM, between about 100 mM and about 2000 mM, between about 200 mM and about 2000 mM, between about 500 mM and about 2000 mM, between about 750 mM and about 2000 mM, between about 100 mM and about 1500 mM, between about 100 mM and about 1000 mM, 100 mM and about 750 mM, between about 100 mM and about 500 mM, between about 100 mM and about 400 mM, between about 100 mM and about 300 mM, between about 100 mM and about 200 mM, 25 between about 100 mM and about 175 mM, between about 100 mM and about 150 mM, between about 125 mM and about 750 mM, between about 125 mM and about 500 mM, between about 125 mM and about 400 mM, between about 125 mM and about 300 mM, between about 125 mM and about 200 mM, between about 125 mM and about 175 mM, between about 125 mM and about 150 mM, between about 150 mM and about 750 mM, between about 150 mM and about 500 mM between about 150 mM and about 400 mM, between about 150 mM and about 300 mM, between about 150 mM and about 200 mM, between about 150 mM and about 175 mM, between about 175 mM and about 750 mM, between about 175 mM and about 500 mM, between about 175 mM and about 400 mM, between about 175 mM and about 300 mM, or between about 175 mM and about 200 mM of the salt. In a further embodiment, the formulation may comprise about 50 mM, about 75 mM, about 80 mM, about 100 mM, about 125 mM, about 150 mM, about 175 mM, about 200 mM, about 300 mM, about 400 mM, about 500 mM, about 600 mM, about 700 mM of the salt, about 800 mM of the salt, about 900 mM of the salt, about 1000 mM of the salt, about 1100 mM of the salt, about 1200 mM of the salt, about 1300 mM of the salt, about 1400 mM of the salt, about 1500 mM of the salt, about 1600 mM of the salt, about 1700 mM of the salt, about 1800 mM of the salt, about 1900 mM of the salt, or about 2000 mM of the salt. In some embodiments, the concentration of the salt in the formulation is at a concentration of 50-2000 mM. In one embodiment, the salt is at a concentration of 100 mM-750 mM. In another embodiment, the salt is at a concentration of 125 mM-250 mM. In certain embodiments, the salt is at a concentration of at least 150 mM. In yet another embodiment, the salt is at a concentration of at least 200 mM. In a further embodiment, the salt is at a concentration of at least 300 mM. In some embodiments, the stable aqueous formulation comprises sodium chloride (NaCl) or potassium chloride (KCl). For example, the stable aqueous formulations herein may comprise at least about 50 mM, at least about 75 mM, at least about 100 mM, at least about 125 mM, at least about 150 mM, at least about 175 mM, at least about 200 mM, at least about 300 mM, at least about 400 mM, at least about 500 mM, at least about 600 mM, at least about 700 mM, at least about 800 mM, at least about 900 mM, at least about 1000 mM, at least about 1100 mM, at least about 1200 mM, at least about 1300 mM, at least about 1400 mM, at least about 1500 mM, at least about 1600 mM, at least about 1700 mM, at least about 1800 mM, or at least about 1900 mM of sodium chloride (NaCl) or potassium chloride (KCl). In a further embodiment, the formulation may comprise between about 50 and about 2000 mM, between about 50 mM and 1500 mM, between about 50 mM and about 1000 mM, between about 50 mM and about 750 mM, between about 50 mM and about 500 mM, between about 50 mM and about 250 mM, between about 50 mM and about 100 mM, between about 100 mM and about 2000 mM, between about 200 mM and about 2000 mM, between about 500 mM and about 2000 mM, between about 750 mM and about 2000 mM, between about 100 mM and about 750 mM, between about 100 mM and about 500 mM, between about 100 mM and about 400 mM, between about 100 mM and about 300 mM, between about 100 mM and about 200 mM, between about 100 mM and about 175 mM, between about 100 mM and about 150 mM, between about 125 mM and about 750 mM, between about 125 mM and about 500 mM between about 125 mM and about 400 mM, between about 125 mM and about 300 mM, between about 125 mM and about 200 mM, between about 125 mM and about 175 mM, between about 125 mM and about 150 mM, between about 150 mM and about 750 mM, between about 150 mM and about 500 mM, between about 150 mM and about 400 mM, between about 150 mM and about 300 mM, between about 150 mM and about 200 mM, between about 150 mM and about 175 mM between about 175 mM and about 750 mM, between about 175 mM and about 500 mM, between about 175 mM and about 400 mM, between about 175 mM and about 300 mM, or between about 175 mM and about 200 mM of sodium chloride (NaCl) or potassium chloride (KCl). In a further embodiment, the formulation may comprise about 50 mM, about 75 mM, about 80 mM, about 100 mM, about 125 mM, about 150 mM, about 175 mM, about 200 mM, about 300 mM, about 400 mM, about 500 mM, about 600 mM, about 700 mM, about 800 mM, about 900 mM, about 1000 mM, about 1100 mM, about 1200 mM, about 1300 mM, about 1400 mM, about 1500 mM, about 1600 mM, about 1700 mM, about 1800 mM, about 1900 mM, or about 2000 mM of sodium chloride (NaCl) or potassium chloride (KCl). In some embodiments, the concentration of sodium chloride or potassium chloride in the formulation is at a concentration of 50-2000 mM. The stable aqueous formulations herein additionally include a carbohydrate, such as a sugar. Carbohydrates that can be used in the formulations herein include, but are not limited to, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and the like. In some embodiments, the sugar is selected from sucrose, trehalose, mannose, maltose, lactose, glucose, raffinose, cellobiose, gentiobiose, isomaltose, arabinose, glucosamine, or fructose. The formulation may alternatively or additionally include a sugar alcohol such as glycol, glycerol, erythritol, threitol, arabitol, xylitol, ribitol, mannitol, sorbitol, dulcitol, iditol, isomalt, maltitol, lactitol or polyglycitol. In one embodiment, the sugar alcohol is xylitol. In some embodiments, the sugar is a non-reducing sugar, e.g., sucrose, trehalose or mannose. In one embodiment, the sugar is sucrose. In another embodiment, the sugar is trehalose. In yet another embodiment, the sugar is mannitol. In a further embodiment, the sugar is sorbitol.

In certain embodiments, the concentration of the carbohydrate (e.g., sugar) in the formulation is chosen from the following ranges: from 1% to 50%, 5% to 50%, 10% to 50%, 15% to 50%, 20% to 50%, 25% to 50%, 30% to 50%, 40% to 50, 45% to 50%, 1% to 40%, 5% to 40%, 5% to 35%, 5% to 30%, 5% to 25%, 5% to 20%, 5% to 15%, 5% to 10%, 1% to 30%, 5% to 30%, 10% to 30%, 15% to 30%, 20% to 30%, 25 to 30%, 1% to 20%, 5% to 20%, 10% to 20%, 15% to 20%, 2% to 10%, 2% to 9%, 2% to 8%, from 2% to 7%, from 2% to 6%, from 2% to 5%, from 2% to 4%, from 2% to 3%, from 2% to 2.5%, from 2.5% to 3%, from 3% to 10%, from 4% to 10%, from 5% to 10%, from 6% to 10%, from 3% to 6%, from 4% to 8%, from 4% to 6%, from 4% to 5%, from 5% to 6%, from 6% to 7%, from 7% to 8%, or more than 8% (weight/volume (w/v)). In certain embodiments, the concentration of the carbohydrate (e.g., sugar) in the formulation is at least 1%, at least 2%, at least 3%, at least 5%, at least 6%, at least 7%, at least 8%, at least 9%, at least 10%, at least 20%, at least 30%, at least 40%, or at least 50% (w/v). In some embodiments, the concentration of the carbohydrate (e.g., sugar) in the formulation is about 1%, about 2%, about 3%, about 4%, about 5%, about 6%, about 7%, about 8%, about 9%, about 10%, about 15%, about 20%, about 25%, about 30%, about 35%, about 40%, about 45%, or about 50% (w/v). In one embodiment, the formulation herein includes sucrose, e.g., at a concentration of at least 5.0% (w/v). In one particular embodiment, the formulation herein includes sucrose, e.g., at a concentration of about 5.0% (w/v).

In certain embodiments, the formulation includes sucrose at a concentration of 1% to 50%, 5% to 50%, 10% to 50%, 15% to 50%, 20% to 50%, 25% to 50%, 30% to 50%, 40% to 50, 45% to 50%, 1% to 40%, 5% to 40%, 5% to 35%, 5% to 30%, 5% to 25%, 5% to 20%, 5% to 15%, 5% to 10%, 1% to 30%, 5% to 30%, 10% to 30%, 15% to 30%, 20% to 30%, 25 to 30%, 1% to 20%, 5% to 20%, 10% to 20%, 15% to 20%, 2% to 10%, 2% to 9%, 2% to 8%, from 2% to 7%, from 2% to 6%, from 2% to 5%, from 2% to 4%, from 2% to 3%, from 2% to 2.5%, from 2.5% to 3%, from 3% to 10%, from 4% to 10%, from 5% to 10%, from 6% to 10%, from 3% to 6%, from 4% to 8%, from 4% to 6%, from 4% to 5%, from 5% to 6%, from 6% to 7%, from 7% to 8%, or more than 8% (weight/volume (w/v)). In certain embodiments, the concentration of sucrose in the formulation is at least 1%, at least 2%, at least 3%, at least 5%, at least 6%, at least 7%, at least 8%, at least 9%, at least 10%, at least 20%, at least 30%, at least 40%, or at least 50% (w/v). In some embodiments, the concentration of sucrose in the formulation is about 2%, about 3%, about 4%, about 5%, about 6%, about 7%, about 8%, about 9%, about 10%, about 15%, about 20%, about 25%, about 30%, about 35%, about 40%, about 45%, or about 50% (w/v).

The stable aqueous formulation further includes a free amino acid, which can be in the L-form, the D-form or any desired mixture of these forms. In one embodiment, free amino acids that can be included in the formulation include, for example, histidine, alanine, arginine, glycine, glutamic acid, serine, threonine, lysine, tryptophan, valine, cysteine and combinations thereof. Some amino acids can stabilize proteins (e.g., stabilized against aggregation or degradation) during manufacturing and/or storage, e.g., through hydrogen bonds, salt bridges, antioxidant properties, or hydrophobic interactions or by exclusion from the protein surface. Amino acids can act as tonicity modifiers or can act to decrease viscosity of the formulation. Free amino acids, such as glutamic acid and histidine, alone or in combination, can also act as buffering agents in aqueous solution in the pH range of 5 to 7.5 (e.g., pH 7.4).

In one embodiment, the free amino acid of the formulation is histidine, arginine, or a combination of histidine and arginine. In one particular embodiment, the free amino acid of the formulation is histidine. In another embodiment, the free amino acid of the formulation is arginine.

In some embodiments, the concentration of the free amino acid in the formulation is at least about 1 mM, at least about 10 mM, at least about 20 mM at least about 25 mM, at least about 50 mM, at least about 100 mM, at least about 150 mM, at least about 200 mM, at least about 250 mM, at least about 300 mM, at least about 350 mM, at least about 400 mM, at least about 500 mM, at least about 600 mM, at least about 700 mM, at least about 800 mM, at least about 900 mM, or at least about 1000 mM. In another embodiment, the formulation may comprise between about 10 and about 1000 mM, between about 10 mM and about 900 mM, between about 10 mM and about 800 mM, between about 10 mM and about 700 mM, between about 10 mM and about 600 mM, between about 10 mM and about 500 mM, between about 10 mM and about 400 mM, between about 10 mM and about 300 mM, between about 10 mM and about 200 mM, between about 10 mM and about 100 mM, between about 100 mM and about 1000 mM, between about 200 mM and about 1000 mM, between about 300 mM and about 1000 mM, between 400 mM and about 1000 mM, between about 400 mM and about 1000 mM, between about 500 mM and about 1000 mM, between about 600 mM and about 1000 mM, between about 700 mM and about 1000 mM, between about 800 mM and about 1000 mM, between about 900 mM and about 1000 mM, between about 1 mM and about 100 mM, between about 10 mM and about 150 mM, between about 25 mM and about 250 mM, between about 25 mM and about 300 mM, between about 25 mM and about 350 mM, between about 25 mM and about 400 mM, between about 50 mM and about 250 mM, between about 50 mM and about 300 mM, between about 50 mM and about 350 mM, between about 50 mM and about 400 mM, between about 100 mM and about 250 mM, between about 100 mM and about 300 mM, between about 100 mM and about 400 mM between about 150 mM and about 250 mM, between about 150 mM and about 300 mM, or between about 150 mM and about 400 mM of an amino acid. In a further embodiment, a formulation comprises about 1 mM, about 20 mM, about 25 mM, about 50 mM, about 100 mM, about 150 mM, about 200 mM, about 250 mM, about 300 mM, about 350 mM, or about 400 mM of an amino acid. In one embodiment, the formulation comprises 100 mM of the free amino acid. In yet another embodiment, the formulation comprises 20 mM of a free amino acid. In another embodiment, the formulation comprises 10-1000 mM of a free amino acid. In one particular embodiment, the formulation comprises about 100 mM of L-arginine. Additionally or alternatively, the formulation may comprise about 20 mM L-histidine (e.g., at pH 7.4).

The formulation can additionally include a buffering agent. As used herein, “buffering agent” refers to a buffer that resists changes in pH by the action of its acid-base conjugate components. Typically, the buffering agent is a salt prepared from an organic or inorganic acid or base. Representative buffering agents include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. In addition, amino acid components can also function in a buffering capacity. Representative amino acid components which may be utilized in the formulations of the invention as buffering agents include, but are not limited to, glycine and histidine. In certain embodiments, the buffering agent is chosen from histidine, citrate, phosphate, glycine, and acetate. In a specific embodiment, the buffering agent is histidine. In another specific embodiment, the buffering agent is citrate. In yet another specific embodiment, the buffering agent is glycine. In some embodiments, the buffering agent includes acetate, succinate, gluconate, histidine, citrate, phosphate, maleate, cacodylate, 2-[N-morpholino]ethanesulfonic acid (MES), bis(2-hydroxyethyl)iminotris[hydroxymethyl]methane (Bis-Tris), N-[2-acetamido]-2-iminodiacetic acid (ADA), 4-(2-hydroxyethyl)-1-piperazineethanesulfonic acid (HEPES), glycylglycine or other organic acid buffers. In one embodiment, the buffering agent herein is L-histidine (e.g., pH 7.4).

In some embodiments, the pH of the formulation is about 5 to about 8.5, about 5.5 to about 8.5, about 6 to about 8.5, about 6.5 to about 8.5, about 7 to about 8.5, about 7.5 to about 8.5, about 5.0 to about 7.5, to about pH 5.5 to about 7.5, to about pH 6.0 to about 7.0, or to a pH of about 6.3 to about 6.5. In one embodiment, examples of buffering agents that alone or in combination, will control the pH in the 5.0 to 7.5 range. In one embodiment, the formulation has a pH of 5-8.5.

In some embodiments, the formulation has a pH between about 5.5 and about 7.5, between about 6.0 and 7.4, between about 6.2 and 7.4, between about 6.4 and 7.4, between about 6.6 and 7.4, between about 6.8 and 7.4, between about 7.0 and 7.4, between about 6.0 and 7.0, between about 6.0 and 6.5, between about 6.0 and 6.3, between about 6.3 and 7.1, or between about 6.4 and 7.0, or between 6.3 and 6.8. In some embodiments, the formulation has a pH at about 5.0, about 5.1, about 5.2, about 5.3, about 5.4, about 5.5, about 5.6, about 5.7 about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In one particular embodiment, the formulation has a pH of about 7.4.

In one aspect, provided herein is a stable aqueous formulation comprising at least 100 mM of a salt, at least 3% w/v of a sugar, a free amino acid, and a targeted active gene editing (TAGE) agent comprising a cell targeting agent and a site-directed modifying polypeptide that recognizes a nucleic acid, wherein the formulation has a pH of about 5 to 8. For example, in one embodiment, the formulation comprises at least 5% sucrose (w/v), at least 200 mM NaCl, at least 10 mM histidine (e.g., L-histidine, pH 7.4), and at least 50 mM arginine. In another embodiment, the formulation comprises 5% sucrose (w/v), 300 mM NaCl, 20 mM histidine (e.g., L-histidine, pH 7.4), and 100 mM arginine.

One or more other pharmaceutically acceptable carriers, excipients or stabilizers such as those described in Remington: The Science and Practice of Pharmacy, 21st Edition, Hendrickson, R. Ed. (2005) may be included in the formulation provided that they do not adversely affect the desired characteristics of the formulation. Acceptable carriers, excipients or stabilizers are nontoxic to recipients at the dosages and concentrations employed and include; additional buffering agents; co-solvents; antioxidants including citrate and cysteine; chelating agents such as EDTA; metal complexes (e.g., Zn-protein complexes); biodegradable polymers such as polyesters; preservatives; container wall lubricants, e.g., silicone, mineral oil, glycerin, or TRIBOGLIDE® (Tribo Film Research, Inc.) perfluoropolyether derivative, for injection ease and/or salt-forming counterions such as sodium.

### III. Improved Gene Editing Methods and Compositions

Provided herein are methods using additives in cell media for gene editing. As described in the examples below, supplementing cell media with certain additives when incubating a site directed-modifying polypeptide, e.g., a TAGE agent, with a cell for gene editing can result in increased uptake of the site directed-modifying polypeptide, e.g., a TAGE agent, resulting in increased gene editing.

For example, provided herein is a method for modifying a nucleic acid in a cell ex vivo, the method comprising contacting at least one cell in a cell medium with a site directed-modifying polypeptide that recognizes a nucleic acid that is within the cell. The nucleic acid may be, for example, in the genome of the cell, or may be trans in the cell. The method includes supplementing the cell medium with a salt and/or a sugar alcohol or a sugar. Other additives are also contemplated herein, including chloroquine.

In other embodiments, the disclosure provides a method of achieving genome editing in a population of cells ex vivo, the method comprising contacting a population of cells in a cell medium with a site directed modifying polypeptide that recognizes a nucleic acid in the genome of cells in the population of cells, wherein the cell medium comprises an effective amount of a salt and/or a sugar alcohol and/or a sugar, such that genome editing is achieved in the population of cells. Other additives are also contemplated herein, including chloroquine.

The amount of additive used is an amount effective to provide gene editing with minimal toxicity on the cells in the cell medium. For example, in one embodiment, an effective amount of the additive, e.g., effective amount of a salt and/or a sugar alcohol and/or a sugar, is an amount whereby gene editing occurs but less than 10%, less than 20%, less than 30%, less than 40%, or less than 50% of the cells die upon contact with the site directed-modifying polypeptide and additive.

Alternatively, in one embodiment, an effective amount of the additive is an amount resulting in at least 10%, at least 11%, at least 12%, at least 13%, at least 14% gene editing. The ability of a site-directed modifying polypeptide to edit a target nucleic acid, e.g., gene, in a target cell can be determined according to methods known in the art, including, for example, phenotypic assays or sequencing assays. In some embodiments, the gene editing assay determines the presence or absence of a marker associated with the gene or nucleic acid of the target cell that is being edited by the TAGE agent. For example, as described in the examples, a CD47 flow cytometry assay can be used to determine the efficacy of a TAGE agent for gene editing. In the CD47 flow cytometry assay, an endogenous CD47 gene sequence in the target cell is targeted by the TAGE agent, where editing is evidenced by a lack of CD47 expression on the cell surface of the target cell. Levels of CD47 can be measured in a population of cells and compared to a control agent (e.g., where a non-targeting guide RNA is used as a negative control) in the same type of target cell.

Other ways in which nucleic acid, e.g., gene, editing activity of a TAGE agent can be determined include sequence based assays, e.g., amplicon sequencing, known in the art. In alternative embodiments, an endogenous sequence in the target cell is targeted by the TAGE agent, where editing is evidenced by an increase in expression of a marker on the cell surface of the target cell or intracellular to account for TdTomato, fluorescent (GFP), etc., reporters. In such embodiments, increases in the level of a marker as detected by flow cytometry, for example, relative to the control indicates gene editing of the TAGE agent. In certain instances, an increase of the cell surface marker of at least 1%, at least 2%, at least 5%, at least 10%, at least 15%, at least 20%, at least 25%, at least 30%, at least 35%, and so forth, relative to a control in a testing assay indicates nucleic acid, e.g., gene, editing by the TAGE agent. In certain instances, an increase in expression of the cell surface marker of at least 2-fold, 3-fold, 4-fold, 5-fold, 6-fold, 7-fold, 8-fold, 9-fold, 10-fold, or more, and so forth, relative to a control in a testing assay indicates nucleic acid, e.g., gene, editing by the TAGE agent. For example, an increase in the expression of a fluorescent marker (e.g., TdTomato fluorescent system) can be used to measure an increase of editing by the TAGE agent. Ranges of the foregoing percentages are also contemplated herein. Other ways in which nucleic acid, e.g., gene, editing activity of a TAGE agent can be determined include sequence based assays known in the art, e.g., amplicon sequencing or T7 endonuclease 1 (T7E1) mismatch detection assays.

The additive, e.g., the salt, the sugar alcohol, or the sugar can either already be in the cell medium when the cells and site directed-modifying polypeptide are combined or it may be added. In certain embodiments, the additive is added to the cell medium prior to contacting the cells with the site directed modifying polypeptide. In other embodiments, the additive is added concurrently with contacting the cells with the site directed modifying polypeptide. In other embodiments, the additive is added after contacting the cells with the site directed modifying polypeptide.

In certain embodiments, the cells are contacted with a site directed-modifying polypeptide in a cell medium comprising a sugar alcohol. Examples of a sugar alcohol that may be used in the methods described herein includes, but is not limited to, erythritol, xylitol, mannitol, and inositol. In one embodiment, the sugar alcohol is glycerol.

Examples of amounts of sugar alcohol that can be used in the cell medium (final concentration in the cell medium) include, but are not limited to, at least about 0.2 M, at least about 0.4 M, at least about 0.8 M, at least about 1.2 M, at least about 1.6 M, at least about 2.0 M, at least about 2.4 M, or at least about 2.6 M of the sugar alcohol. In other embodiments, the cell medium is adjusted to a sugar alcohol amount of 0.2 M to 2.6 M, 0.4 M to 2.4 M, 0.4 M to 1 M, 1 M to 2.5 M, 0.2 M to 0.5 M, 0.5 M to 1 M, 1 M to 1.5 M, 1.5 M to 2 M, or 2 M to 2.5 M, of the sugar alcohol.

In other instances, the cell medium comprises at least about 4% w/v, at least about 5% w/v, at least about 7.5% w/v, at least about 10% w/v, at least about 12.5% w/v, at least about 15% w/v, 4% w/v to 15% w/v, 10% w/v to 15%, 4% w/v to 6% w/v, 4% w/v to 8% w/v, 8% w/v to 12%, 6% w/v to 8% w/v, 8% w/v to 10% w/v, 10% w/v to 12% w/v, 12% w/v to 14% w/v, or 14% w/v to 16%, or 5% w/v to 10%, w/v of the sugar alcohol.

Examples of amounts of sugar (e.g., sucrose) that can be used in the cell medium (final concentration in the cell medium) include, but are not limited to, at least about 0.2 M, at least about 0.4 M, at least about 0.8 M, at least about 1.2 M, at least about 1.6 M, at least about 2.0 M, at least about 2.4 M, or at least about 2.6 M of the sugar (e.g., sucrose). In other embodiments, the cell medium is adjusted to a sugar alcohol amount of 0.2 M to 2.6 M, 0.4 M to 2.4 M, 0.4 M to 1 M, 1 M to 2.5 M, 0.2 M to 0.5 M, 0.5 M to 1 M, 1 M to 1.5 M, 1.5 M to 2 M, or 2 M to 2.5 M of the sugar (e.g., sucrose).

In other instances, the cell medium comprises at least about 4% w/v, at least about 5% w/v, at least about 7.5% w/v, at least about 10% w/v, at least about 12.5% w/v, at least about 15% w/v, 4% w/v to 15% w/v, 10% w/v to 15%, 4% w/v to 6% w/v, 4% w/v to 8% w/v, 8% w/v to 12%, 6% w/v to 8% w/v, 8% w/v to 10% w/v, 10% w/v to 12% w/v, 12% w/v to 14% w/v, or 14% w/v to 16%, or 5% w/v to 10%, w/v of the sugar (e.g., sucrose).

Examples of amounts of salt (e.g., sodium chloride) that can be used in the cell medium (final concentration in the cell medium) include, but are not limited to, at least about 150 mM, at least about 175 mM, at least about 185 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, or at least about 450 mM of the salt. In other embodiments, the cell medium is adjusted to a salt concentration (final in the cell medium) of 100 mM to 500 mM, 185 mM to 450 mM, 100 mM to 250 mM, 250 mM to 500 mM, 150 mM to 200 mM, 200 mM to 250 mM, 250 mM to 300 mM, 300 mM to 350 mM, 350 mM to 400 mM, 400 mM to 450 mM, or 450 mM to 500 mM of the salt.

Examples of amounts of chloroquine that can be used in the cell medium (final concentration in the cell medium) include, but are not limited to, at least 30 μM, at least 50 μM, at least 80 μM at least 100 μM, 1-200 μM, 10-150 μM, or 25-100 μM chloroquine. In other embodiments, the cell medium is adjusted to a chloroquine (final in the cell medium) of 25-50 μM, 50-75 μM, or 75-100 μM chloroquine.

In some embodiments, the cell medium further includes an additional additive, such as an additive that enhances cellular uptake of the RNP (e.g., nystatin, EIPA, DMA, as demonstrated in Examples 18 and 19). In some embodiments, the additional additive is an inhibitor of NHE1 (e.g., EIPA or DMA, e.g., see Example 18).

In some embodiments, the cell medium further includes nystatin. In one embodiment, the cell medium comprises at least about 1 μM, at least about 10 μM, at least about 20 μM, at least about 30 μM, at least about 40 μM, at least about 50 μM, at least about 60 μM, at least about 70 μM, at least about 80 μM, at least about 90 μM, at least about 100 μM, at least about 110 μM, or at least about 120 μM of nystatin. In one embodiment, the cell medium comprises 1 μM-120 μM, 20 μM-120 μM, 30 μM-120 μM, 40 μM-120 μM, 50 μM-120 μM, 60 μM-120 μM, 70 μM-120 μM, 80 μM-120 μM, 90 μM-120 μM, 100 μM-120 μM, 10 μM-110 μM, 10 μM-100 μM, 10 μM-90 μM, 10 μM-80 μM, 10 μM-70 μM, 10 μM-60 μM, 10 μM-50 μM, 10 μM-40 μM, 10 μM-30 μM, or 10 μM-20 μM of nystatin. In one embodiment, the cell medium comprises 100 μM to 110 μM of nystatin. In one embodiment, the cell medium comprises about 108 μM of nystatin.

In some embodiments, the cell medium further includes EIPA (5-(N-ethyl-N-isopropyl)-amiloride). In one embodiment, the cell medium comprises at least about 1 μM, at least about 10 μM, at least about 20 μM, at least about 30 μM, at least about 40 μM, at least about 50 μM, at least about 60 μM, at least about 70 μM, at least about 80 μM, at least about 90 μM, or at least about 100 μM of EIPA. In one embodiment, the cell medium comprises 1 μM-120 μM, 20 μM-120 μM, 30 μM-120 μM, 40 μM-120 μM, 50 μM-120 μM, 60 μM-120 μM, 70 μM-120 μM, 80 μM-120 μM, 90 μM-120 μM, 100 μM-120 μM, 10 μM-110 μM, 10 μM-100 μM, 10 μM-90 μM, 10 μM-80 μM, 10 μM-70 μM, 10 μM-60 μM, 10 μM-50 μM, 10 μM-40 μM, 10 μM-30 μM, or 10 μM-20 μM of EIPA. In one embodiment, the cell medium comprises 90 μM to 110 μM of EIPA. In one embodiment, the cell medium comprises about 100 μM of EIPA.

In some embodiments, the cell medium further includes DMA (5-(N,N-dimethyl)-amiloride (5-(N-ethyl-N-isopropyl)-amiloride)). In one embodiment, the cell medium comprises at least about 1 μM, at least about 10 μM, at least about 20 μM, at least about 30 μM, at least about 40 μM, at least about 50 μM, at least about 60 μM, at least about 70 μM, at least about 80 μM, at least about 90 μM, or at least about 100 μM of DMA. In one embodiment, the cell medium comprises 1 μM-120 μM, 20 μM-120 μM, 30 μM-120 μM, 40 μM-120 μM, 50 μM-120 μM, 60 μM-120 μM, 70 μM-120 μM, 80 μM-120 μM, 90 μM-120 μM, 100 μM-120 μM, 10 μM-110 μM, 10 μM-100 μM, 10 μM 90 μM, 10 μM-80 μM, 10 μM-70 μM, 10 μM-60 μM, 10 μM-50 μM, 10 μM-40 μM, 10 μM-30 μM, or 10 μM-20 μM of DMA. In one embodiment, the cell medium comprises 90 μM to 110 μM of EIPA. In one embodiment, the cell medium comprises about 100 μM of DMA.

Preferably, the gene editing methods using the additives described herein are performed ex vivo.

### IV. Detection and Production of RNPs

Additionally, provided herein is a method to produce and detect formulations having efficient RNP formation and reduced levels of free gRNA. When producing RNPs, some samples include free gRNA and/or nucleic acid-guided nucleases (e.g., Cas9) in addition to the properly formed RNP. As demonstrated in Example 2, free gRNA in the sample can have an aberrant elution profile under certain elution conditions, which can interfere with the resolution and detection of RNPs in a sample. Such samples may also have undesirable characteristics such as altered immunogenicity or toxicity due to the properties of free RNA or free Cas protein that differs from those of the RNP. During formation of these complexes, it therefore important to have methods to detect the efficiency of RNP formation, for example by detecting the presence of unbound gRNA or nucleic acid-guided nuclease species alongside the RNP complex.

The methods provided herein enable resolution of RNPs (e.g., TAGE agents) from unbound gRNA or nucleases, which is useful for methods of RNP detection and production. Accordingly, in one aspect, provided herein is a method of determining the amount of an RNP in a sample comprising the RNP and an unbound guide RNA (gRNA). In another aspect, provided herein is a method of producing a composition comprising a ribonucleoprotein (RNP) having reduced levels of an unbound guide RNA (gRNA).

To help resolve RNPs (e.g., TAGE agents) from unbound gRNA, in some embodiments, the methods herein include pre-treatment of the gRNA (e.g., to generate refolded gRNA) and/or the use of buffers (e.g., buffers that include Mg²⁺) that promote optimal separation of unbound gRNA from RNPs. For example, when using Size Exclusion Chromatography resin to separate the unbound gRNA from the RNPs, the methods herein may enable the majority (e.g., greater than 50%, greater than 60%, greater than 70%, greater than 80%, greater than 90%, greater than 95%, greater than 98%, or greater than 99%) of the unbound gRNA to elute from the column as a single peak from the Size Exclusion Chromatography resin. In preferred embodiments, the unbound gRNA elutes from the SEC column as a single peak at a different elution time than the RNPs, thereby resolving the RNPs from the unbound gRNAs.

In one embodiment, the RNP (e.g., TAGE agent) detection or production method provided herein includes a pre-treatment step to generate refolded gRNA. As demonstrated in Examples 2 and 3, refolded gRNA can elute as a single peak from a SEC column, whereas gRNA that has not undergone this refolding process tends to elute as multiple peaks over a wider time span and can interfere with detection of RNPs. To refold gRNA, the gRNA is heated (e.g., to a temperature that at least partially denature the gRNA) and then subsequently cooled (e.g., to a temperature that allows the gRNA to renature). For example, in some embodiments, the gRNA can be refolded by heating the gRNA to a temperature of at least 70° C. and then cooling the gRNA at or below room temperature (e.g., 2° C.-25° C.).

Accordingly, in some embodiments of the methods herein, the gRNA is pre-treated under conditions effective to refold the gRNA. In some such embodiments, the gRNA is refolded by heating the gRNA to a temperature of at least 60° C. (e.g., 60° C., 65° C., 70° C., 75° C., 80° C., 90° C., 95° C., or more) and cooling the gRNA at ambient temperature (e.g., 20° C., 21° C., 22° C., 23° C., 24° C., or 25° C.). In some embodiments, the gRNA is refolded by heating the gRNA to a temperature of at least 60° C. (e.g., 60° C., 65° C., 70° C., 75° C., 80° C., 90° C., 95° C., or more) and cooling the gRNA at a temperature of 10° C.-18° C. (e.g., 10° C., 12° C., 14° C., 16° C., or 18° C.). In some embodiments, the gRNA is refolded by heating the gRNA to a temperature of at least 60° C. (e.g., 60° C., 65° C., 70° C., 75° C., 80° C., 90° C., 95° C., or more) and cooling the gRNA at a temperature of 2° C.-8° C. (e.g., 2° C., 4° C., 6° C., or 8° C.). In one embodiment, the gRNA is refolded by heating the gRNA to a temperature of 60° C. to 95° C. In certain embodiments, the gRNA is heated for at least 1 minute (e.g., 1 minute, 2 minutes, 3 minutes, 4 minutes, 5 minutes, 6 minutes, 7 minutes, 8 minutes, 10 minutes, or more than 10 minutes). In some embodiments, the gRNA is cooled for at least 1 minute (e.g., 1 minute, 2 minutes, 3 minutes, 4 minutes, 5 minutes, 6 minutes, 7 minutes, 8 minutes, 10 minutes, or more than 10 minutes).

In one embodiment, the gRNA is refolded by heating the gRNA for at least 1 minute at 70° C. and allowing the gRNA to cool to ambient temperature (e.g., 20° C.-25° C.) at rate of less than 3° C. per minute. In one embodiment, the gRNA is refolded by heating the gRNA for at least 1 minute at 70° C. and allowing the gRNA to cool to ambient temperature (e.g., 20° C.-25° C.) at rate of about 1° C. to about 3° C. per minute. In one embodiment, the gRNA is refolded by heating the gRNA for at least 1 minute at 70° C. and allowing the gRNA to cool to ambient temperature (e.g., 20° C.-25° C.) at rate of about 2° C. to about 3° C. per minute. In one embodiment, the gRNA is refolded by heating the gRNA for at least 1 minute at 70° C. and allowing the gRNA to cool to ambient temperature (e.g., 20° C.-25° C.) at rate of about 0.5° C. to about 3° C. per minute. In one embodiment, the gRNA is refolded by holding the gRNA for at least 1 minute at 70° C. and incubating the gRNA at ambient temperature for 10 minutes.

In instances where the method utilizes gRNA, the method may entail a pretreatment step to prepare the refolded gRNA prior to preparing the RNPs. In some embodiments, the pretreatment or refolding step is completed less than 72 hours (e.g., less than 1 hour, less than 2 hours, less than 3 hours, less than 4 hours, less than 5 hours, less than 6 hours, less than 8 hours, less than 10 hours, less than 12 hours, less than 24 hours, less than 36 hours, less than 48 hours, less than 60 hours, less than 72 hours, 1 minutes-24 hours, 24 hours to 48 hours, 48 hours to 72 hours, 20-24 hours, 18-22 hours, 15-20 hours, 14-18 hours, 10-15 hours, 8-12 hours, 5-10 hours, 4-8 hours, 1-2 hours, 30 min-60 min, 15-45 min, 10-15 min, 1-10 min) prior to preparation of the RNPs, wherein the RNPs are stored at 2-8° C. (e.g., in aqueous buffer at neutral pH). In some embodiments, the pretreatment or refolding step is completed less than 4 hours (e.g., less than 10 minutes, less than 15 minutes, less than 30 minutes, less than 45 minutes, less than 1 hour, less than 2 hours, less than 3 hours, or less than 4, hours, 1-4 hours, 1-2 hours, 30 min-60 min, 15-45 min, 10-15 min, 1-10 min) prior to preparation of the RNPs, wherein the RNPs are stored at less than or equal to 37° C. (e.g., in aqueous buffer at neutral pH). In some embodiments, the pretreatment or refolding step is performed less than 24 hours (e.g., less than 20 hours, less than 18 hours, less than 16 hours, less than 14 hours, less than 12 hours, less than 10 hours, less than 8 hours, less than 6 hours, less than 4 hours, less than 2 hours, less than 1 hour, less than 45 minutes, less than 30 minutes, less than 15 minutes, less than 10 minutes, less than 5 minutes, less than 2 minutes, or less than 1 minute) prior to preparation of the RNPs.

To prepare RNPs used in the methods provided herein, gRNA can be incubated with the RNA-guided nuclease such that complexes form between the RNA-guided nuclease gRNA to form RNPs. In some embodiments, the gRNA is refolded gRNA, as described above. During this process, some gRNA may remain unbound. In some embodiments, an excess of the gRNA relative to the RNA-guided nuclease forms an unbound gRNA. gRNA that does not bind to the RNA-guided nuclease can be separated in accordance with the methods disclosed herein to detect and produce RNPs in the sample.

Accordingly, in one aspect, provided herein is a method of producing a composition comprising a ribonucleoprotein (RNP) having reduced levels of an unbound guide RNA (gRNA), the method comprising pretreating a gRNA under conditions effective to refold the gRNA, thereby generating a refolded gRNA; combining an RNA-guided nuclease with the refolded gRNA to form an RNP, wherein a portion of the refolded gRNA does not bind to the RNA-guided nuclease and thereby forms an unbound gRNA; separating the RNPs from the unbound gRNA; and collecting one or more fractions that comprise the RNP but do not detectably comprise the unbound gRNA, thereby producing a composition comprising an RNP having a reduced amount of the unbound gRNA.

In another aspect, provided herein is a method of determining the amount of a ribonucleoprotein (RNP) in a sample comprising the RNP and an unbound guide RNA (gRNA), the method comprising: pretreating a gRNA under conditions effective to refold the gRNA, thereby generating a refolded gRNA; combining an RNA-guided nuclease and the refolded gRNA to form an RNP, wherein a portion of the refolded gRNA does not bind to the RNA-guided nuclease and thereby forms an unbound gRNA; separating the RNP from the unbound gRNA; and determining the amount of the RNP in the sample.

A variety of separation techniques can be used in the detection and production methods herein. In some embodiments, the separation step can include, for example, chromatography (e.g., liquid chromatography such as HPLC, Ultra Performance Liquid Chromatography (UPLC), Ultra High Performance Liquid Chromatography (UHPLC), nano-LC, in particular normal phase chromatography, reversed phase chromatography, ion-exchange chromatography, size-exclusion chromatography, affinity chromatography, hydrophobic interaction chromatography or hydrophilic interaction chromatography) or electrophoresis (e.g., capillary electrophoresis). In one embodiment, the separation step involves Size Exclusion Chromatography (SEC).

The amounts of RNPs and their constituents can be determined using a variety of detection techniques, such as UV/Vis spectrophotometry, fluorescence spectroscopy, refractive index detection, light scattering, or mass spectrometry. Further, in some embodiments, the method further comprises determining the relative amount of RNP and unbound gRNA in the sample by comparing a signal produced by the RNP to a signal produced by the unbound gRNA using a detection technique. The separation approach, in some instances, can be coupled with the detection step. For example, when SEC is coupled with light scattering, viscometer, and/or concentration detectors (known as triple detection), it can measure absolute molecular weight, molecular size, and intrinsic viscosity and generate information on macromolecular structure, conformation, aggregation and branching of the sample.

In some embodiments, size exclusion chromatography-high pressure liquid chromatography (alternatively referred to herein as SEC-HPLC or SEC) can be used to separate RNPs from unbound gRNAs in the detection and production methods herein.

In certain embodiments, the levels of RNPs, unbound gRNAs, and/or unbound RNA guided nucleases are analyzed. In various embodiments set forth herein, size exclusion chromatography (SEC) can be used to determine the relative level of RNPs, unbound gRNAs, and/or unbound RNA-guided nuclease present in the sample. The SEC method provides size-based separation of RNPs, unbound gRNAs, and/or unbound RNA. Test samples and reference standards can be analyzed using commercially available SEC columns, using an appropriate buffer. The method can involve use of an initial loading buffer (i.e., a first buffer) comprising the RNPs, unbound gRNA, and/or unbound RNA-guided nucleases, and a mobile phase (i.e., a second buffer) that is used to flow through the chromatography system, moving the materials to be separated at different rates over the stationary phase (i.e., the SEC resin).

For example, in some embodiments, SEC analysis can be performed using a column having a pore size of about 300 Å and a pore diameter of about 2.7 μm, such as an AdvanceBio SEC column (Agilent). In one embodiment, the SEC analysis comprises injecting a sample onto an AdvanceBio SEC column with a loading buffer (e.g., 20 mM HEPES pH 7.5, 200 mM KCl, 1 mM MgCl₂, and 5% (v/v) glycerol), and running the sample over the SEC resin with a running buffer (e.g., 25 mM MES pH 6.0, 500 mM NaCl, and 1 mM MgCl₂), wherein the elution of protein species is monitored at 260 nm (16 nm bandpass) minus 340 nm (16 nm bandpass). The column, buffer, and flow rate can be selected to maximize peak resolution. The main peak(s) and the total peak area are then measured.

To determine or measure the amount of the RNP in the sample, elution of the various species in the sample can be monitored (e.g., via a diode array detector to record the ultraviolet and visible (UV/Vis) absorption spectra of samples that are passing through the high-pressure liquid chromatograph). For example, absorbance of the eluting fractions can be detected based on absorbance at 260 nm (16 nm bandpass) minus 340 nm (16 nm bandpass)). In some embodiments, the eluting fractions are detected at an absorbance of 230 nm, 260 nm, and/or 280 nm, or ratios thereof (e.g., to infer the presence of free protein co-eluting with free gRNAs. In some embodiments, the method further involves fluorescence detection of tryptophan residues (e.g., 290+/−10 nm excitation, 340 nm long pass emission) to detect protein with no interference from RNA, and thus infer the fraction of free protein by the ratio between peak areas in the fluorescence channel.

The area under the peak corresponding to RNPs, unbound gRNAs, and/or unbound RNA-guided nuclease can then be measured relative to the total peak area to determine the percent level of each species in the sample. The percent RNPs (i.e., complexes of a nucleic acid-guided nuclease and a gRNA), the percent unbound gRNA, and/or the percent unbound nucleic acid-guided nuclease are then reported.

Accordingly, in one aspect, provided herein is a method of determining the amount of a ribonucleoprotein (RNP) in a sample comprising the RNP and an unbound guide RNA (gRNA), the method comprising: loading the sample comprising the RNP and the unbound gRNA onto a size exchange chromatography (SEC) column equilibrated in the presence of a first buffer, wherein the RNP comprises gRNA bound to an RNA-guided nuclease, and wherein a portion of the refolded gRNA does not bind to the RNA-guided nuclease and thereby forms an unbound gRNA, and wherein the RNP is in the presence of a second buffer; separating the RNP from the unbound gRNA on the SEC, such that the RNP and the unbound gRNA elute from the column at different times; and determining the amount of the RNP that elutes from the SEC column.

Fractions that elute from the SEC resin that include the desired species (e.g., an RNP) can be collected to produce a sample of RNPs. Before pooling the fractions, the fractions can be further analyzed with additional analytical devices after collecting fractions that elute from the SEC resin. For example, fractions that elute from the SEC column can be analyzed using UV/Vis spectrophotometry, fluorescence spectroscopy, refractive index detection, light scattering, or mass spectrometry to confirm the identity, concentration, and stability of the species in the fractions. Fractions having the desired properties can then be collected for use, for example, in a formulation.

Using the methods herein, unbound gRNA in a sample can be resolved from RNPs using SEC. By ensuring that the majority of unbound gRNA elute at a different elution time than properly formed RNPs, fractions comprising RNPs and low levels or undetectable levels of unbound gRNA can be collected, thereby enabling production of a composition of RNPs having reduced levels of unbound gRNA (e.g., relative to the starting sample).

Accordingly, in another aspect, provided herein is a method of producing a composition comprising a ribonucleoprotein (RNP) having reduced levels of an unbound gRNA, the method comprising: loading a sample comprising the RNP and the unbound gRNA onto a size exchange chromatography (SEC) column equilibrated in the presence of a first buffer, wherein the RNP comprises a gRNA bound to an RNA-guided nuclease, and wherein a portion of the refolded gRNA does not bind to the RNA-guided nuclease and thereby forms an unbound gRNA, and wherein the RNP is in the presence of a second buffer; separating the RNP and the unbound gRNA on the SEC column, such that the RNP and the majority of unbound gRNA elute from the column at different times; collecting one or more fractions that comprise the RNP, thereby producing a composition comprising a ribonucleoprotein (RNP) having a reduced amount of the unbound gRNA. In some embodiments, the collection step comprises collecting one or more fractions that comprise the RNP but do not detectably comprise the unbound gRNA. In some embodiments, the method produces a composition comprising the RNPs having reduced levels of the unbound gRNA relative to the levels of the unbound gRNA in the starting sample.

In some embodiments, the first and second buffer have different compositions. In other embodiments, the first and second buffer are the same. In some embodiments, both the loading buffer and running buffer include MgCl₂.

In some embodiments, the second buffer is a loading buffer.

In one embodiment, the second buffer (i.e., loading buffer) comprises potassium chloride. In some embodiments, the second buffer includes 50 mM-750 mM KCl (e.g., 50 mM, 60 mM, 70 mM, 80 mM, 90 mM, 100 mM, 125 mM, 150 mM, 175 mM, 200 mM, 250 mM, 300 mM, 350 mM, 400 mM, 450 mM, 500 mM, 550 mM, 600 mM, 650 mM, 700 mM, or 750 mM KCl). In some embodiments, the second buffer includes 150 mM-250 mM KCl. In certain embodiments, the second buffer includes 200 mM KCl.

In one embodiment, the second buffer (i.e., loading buffer) comprises potassium chloride. In some embodiments, the second buffer includes 50 mM-750 mM NaCl (e.g., 50 mM, 60 mM, 70 mM, 80 mM, 90 mM, 100 mM, 125 mM, 150 mM, 175 mM, 200 mM, 250 mM, 300 mM, 350 mM, 400 mM, 450 mM, 500 mM, 550 mM, 600 mM, 650 mM, 700 mM, or 750 mM NaCl). In some embodiments, the second buffer includes 150 mM-250 mM NaCl. In certain embodiments, the second buffer includes 200 mM NaCl.

In one embodiment, the second buffer comprises magnesium chloride (MgCl₂). In some embodiments, the second buffer includes 0.1 mM to 10 mM MgCl₂ (e.g., 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 3 mM, 4 mM, 5 mM, 6 mM, 7 mM, 8 mM, 9 mM, or 10 mM MgCl₂). In some embodiments, the second buffer includes 0.5 mM-2 mM MgCl₂. In certain embodiments, the second buffer includes 1 mM MgCl₂.

In one embodiment, the second buffer comprises a buffering agent, such as HEPES. In exemplary embodiments, the second buffer includes 1 mM-50 mM of the buffering agent (e.g., 1 mM, 2 mM, 4 mM, 5 mM, 6 mM, 8 mM, 10 mM, 12 mM, 14 mM, 15 mM, 16 mM, 18 mM, 20 mM, 22 mM, 24 mM, 25 mM, 26 mM, 28 mM, 30 mM, 32 mM, 34 mM, 35 mM, 36 mM, 38 mM, 40 mM, 42 mM, 44 mM, 45 mM, 46 mM, 48 mM, or 50 mM of the buffering agent). In some embodiments, the second buffer includes 20-30 mM of the buffering agent. In certain embodiments, the second buffer includes 20 mM buffering agent.

In certain embodiments, the second buffer comprises HEPES. In exemplary embodiments, the second buffer includes 1 mM-50 mM of HEPES (e.g., 1 mM, 2 mM, 4 mM, 5 mM, 6 mM, 8 mM, 10 mM, 12 mM, 14 mM, 15 mM, 16 mM, 18 mM, 20 mM, 22 mM, 24 mM, 25 mM, 26 mM, 28 mM, 30 mM, 32 mM, 34 mM, 35 mM, 36 mM, 38 mM, 40 mM, 42 mM, 44 mM, 45 mM, 46 mM, 48 mM, or 50 mM of HEPES). In some embodiments, the second buffer includes 20-30 mM of HEPES. In certain embodiments, the second buffer includes 20 mM HEPES.

In one embodiment, the second buffer comprises a glycerol. In exemplary embodiments, the second buffer includes at least 1% ((volume of solute)/(volume of solution) or “v/v”) glycerol (e.g., 1%, 2%, 3%, 4%, 5%, 6%, 7%, 8%, 9%, 10%, or more than 10% (v/v) glycerol). In some embodiments, the second buffer includes 4-6% (v/v) glycerol. In certain embodiments, the second buffer includes 5% (v/v) glycerol.

In some embodiments, the second buffer is at a pH at or above pH 6.5, e.g., pH 6.5-8.5, pH 7.0-7.5, pH 6.8-7.4, etc. In some embodiments, the second buffer is at a pH of about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5. In some embodiments, the second buffer is at a pH of 7.0 to 8.0. In one embodiment, the second buffer is at a pH of 7.5.

In one embodiment, the second buffer (i.e., loading buffer) comprises a buffering agent (e.g., HEPES, pH 7.5), potassium chloride, and magnesium chloride. In some embodiments, the second buffer comprises 20 mM HEPES pH 7.5, 200 mM KCl, and 1 mM MgCl₂. In certain embodiments, the second buffer comprises 20 mM HEPES pH 7.5, 200 mM KCl, 1 mM MgCl₂, and 5% (v/v) glycerol.

In some embodiments, the first buffer is a running buffer.

In one embodiment, the first buffer (i.e., running buffer) comprises sodium chloride (NaCl). In some embodiments, the second buffer includes 50 mM-750 mM NaCl (e.g., 50 mM, 60 mM, 70 mM, 80 mM, 90 mM, 100 mM, 125 mM, 150 mM, 175 mM, 200 mM, 250 mM, 300 mM, 350 mM, 400 mM, 450 mM, 500 mM, 550 mM, 600 mM, 650 mM, 700 mM, or 750 mM NaCl). In some embodiments, the second buffer includes 150 mM-250 mM NaCl. In certain embodiments, the second buffer includes 200 mM NaCl.

In one embodiment, the first buffer (i.e., running buffer) comprises potassium chloride (KCl).

In some embodiments, the first buffer includes 50 mM-750 mM KCl (e.g., 50 mM, 60 mM, 70 mM, 80 mM, 90 mM, 100 mM, 125 mM, 150 mM, 175 mM, 200 mM, 250 mM, 300 mM, 350 mM, 400 mM, 450 mM, 500 mM, 550 mM, 600 mM, 650 mM, 700 mM, or 750 mM KCl). In some embodiments, the second buffer includes 150 mM-250 mM KCl. In certain embodiments, the first buffer includes 200 mM KCl.

In one embodiment, the first buffer comprises magnesium chloride (MgCl₂). In some embodiments, the second buffer includes 0.1 mM to 10 mM MgCl₂ (e.g., 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 3 mM, 4 mM, 5 mM, 6 mM, 7 mM, 8 mM, 9 mM, or 10 mM MgCl₂). In some embodiments, the first buffer includes 0.5 mM-2 mM MgCl₂. In certain embodiments, the second buffer includes 1 mM MgCl₂.

In one embodiment, the first buffer comprises a buffering agent, such as MES. In exemplary embodiments, the first buffer includes 1 mM-50 mM of the buffering agent (e.g., 1 mM, 2 mM, 4 mM, 5 mM, 6 mM, 8 mM, 10 mM, 12 mM, 14 mM, 15 mM, 16 mM, 18 mM, 20 mM, 22 mM, 24 mM, 25 mM, 26 mM, 28 mM, 30 mM, 32 mM, 34 mM, 35 mM, 36 mM, 38 mM, 40 mM, 42 mM, 44 mM, 45 mM, 46 mM, 48 mM, or 50 mM of the buffering agent). In some embodiments, the second buffer includes 20-30 mM of the buffering agent. In certain embodiments, the first buffer includes 20 mM buffering agent.

In certain embodiments, the first buffer comprises MES. In exemplary embodiments, the second buffer includes 1 mM-50 mM of MES (e.g., 1 mM, 2 mM, 4 mM, 5 mM, 6 mM, 8 mM, 10 mM, 12 mM, 14 mM, 15 mM, 16 mM, 18 mM, 20 mM, 22 mM, 24 mM, 25 mM, 26 mM, 28 mM, 30 mM, 32 mM, 34 mM, 35 mM, 36 mM, 38 mM, 40 mM, 42 mM, 44 mM, 45 mM, 46 mM, 48 mM, or 50 mM of MES). In some embodiments, the second buffer includes 20-30 mM of MES. In certain embodiments, the first buffer includes 25 mM MES.

In some embodiments, the first buffer is at a pH between pH 5.0 and 6.5, e.g., pH 5.0-6.5, pH 5.5-6.5, pH 5.8-6.4, pH 6.0-6.2, etc. In some embodiments, the second buffer is at a pH of about 5.0, 5.1, 5.2, 5.3, 5.4 5.5, 5.6, 5.7, 5.8, 5.9, 6.0, 6.1, 6.2, 6.3, 6.4, or 6.5. In some embodiments, the second buffer is at a pH of 5.5 to 6.5. In one embodiment, the first buffer is at a pH of 6.0.

In some embodiment, the first buffer (i.e., the running buffer) comprises a buffering agent (e.g., MES, pH 6.0), sodium chloride, and magnesium chloride. In one embodiment, the first buffer comprises 25 mM MES pH 6.0, 500 mM NaCl, 1 mM MgCl₂.

The RNP produced or detected by the present methods can include a TAGE agent described herein (e.g., see Section III). A TAGE agent comprises a cell targeting agent and a site-directed modifying polypeptide that recognizes a nucleic acid. In some embodiments, the site-directed modifying polypeptide that recognizes a nucleic acid is a nucleic acid-guided nuclease, such as an RNA-guided nuclease. For example, in some embodiments, the RNA-guided nuclease is Class 2 Cas polypeptide, such as a Type II Cas polypeptide (e.g., Cas9) or a Type V Cas polypeptide (e.g., Cas12). In some embodiments, the formulation further comprises a guide nucleic acid (gNA), wherein the gNA and the nucleic acid-guided nuclease form a nucleoprotein. In some embodiments, the guide nucleic acid is a guide RNA (gRNA), the nucleic acid-guided nuclease is an RNA-guided nuclease, and the gRNA and RNA-guided nuclease form a ribonucleoprotein. The cell targeting agent of the TAGE agent can be, for example, a ligand, a cell penetrating peptide, or an antigen-binding polypeptide, including any of those set forth in Section III.

### V. TAGE Agents

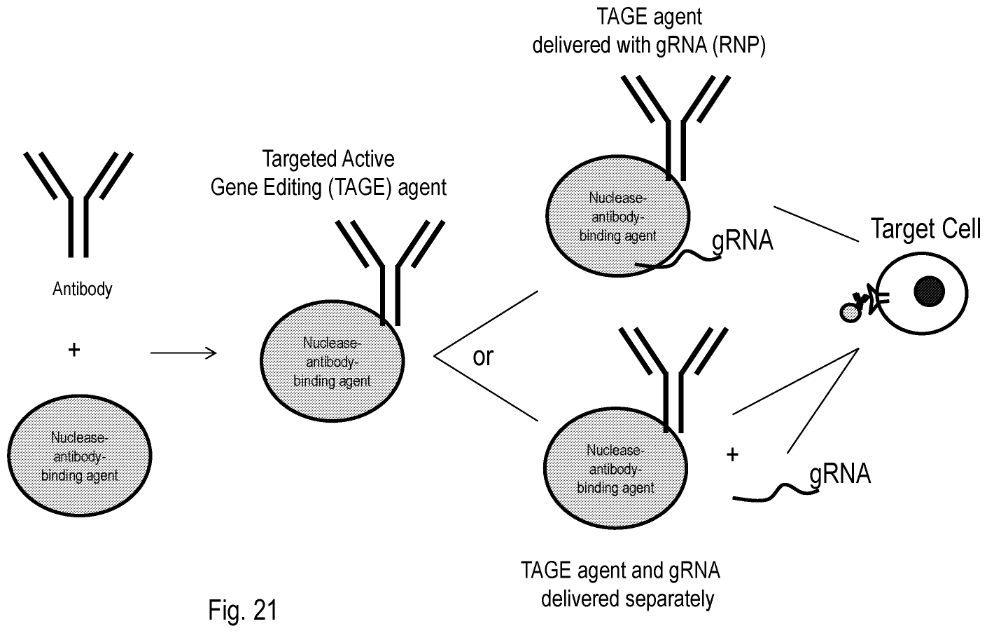

The RNPs of the present formulations and methods can comprise a targeted active gene editing (TAGE) agent that is useful for delivering a gene editing polypeptide (i.e., a site-directed modifying polypeptide) to a target cell. In some embodiments, the TAGE agent can be a biologic. In particular embodiments, the site-directed modifying polypeptide contains a conjugation moiety that allows the protein to be conjugated to an extracellular cell membrane binding moiety (e.g., an antigen binding protein, ligand, or cell penetrating peptide (CPP), or combinations thereof) that binds to an antigen associated with the extracellular region of a cell membrane or otherwise increases cellular or nuclear internalization of the site-directed modifying polypeptide. In the case of TAGE agent that include a ligand or antigen-binding protein, this target specificity allows for delivery of the site-directed modifying polypeptide only to cells displaying the antigen (e.g., hematopoietic stem cells (HSCs), hematopoietic progenitor stem cells (HPSCs), natural killer cells, macrophages, DC cells, non-DC myeloid cells, B cells, T cells (e.g., activated T cells), fibroblasts, or other cells). Such cells may be associated with a certain tissue or cell-type associated with a disease. The TAGE agent thus provides a means by which the genome of a target cell can be modified. TAGE agents are further described in International Publication Nos. WO 2020/198151 and WO 2020/198160, as well as U.S. application Ser. Nos. 17/480,913 and 17/481,056, which are each hereby incorporated by reference. A representation of a TAGE is shown in FIG. 21.

In one embodiment, a TAGE agent comprises a nucleic acid-guided endonuclease (e.g., RNA-guided endonuclease or DNA-guided endonuclease), such as Cas9, that recognizes a CRISPR sequence, and an antigen binding protein that specifically binds to an extracellular molecule (e.g., protein, glycan, lipid) localized on a target cell membrane. Examples of antigen binding proteins that can be used in the TAGE agent of the invention include, but are not limited to, an antibody, an antigen-binding portion of an antibody, or an antibody mimetic. The types of antigen binding proteins that can be used in the compositions and methods described herein are described in more detail in Section V.

In another embodiment, a TAGE agent comprises a nucleic acid-guided endonuclease (e.g., RNA-guided endonuclease or DNA-guided endonuclease), such as Cas9, that recognizes a CRISPR sequence, and a ligand that specifically binds to an extracellular molecule (e.g., protein, glycan, lipid) localized on a target cell membrane. Examples of ligands that can be used in the compositions and methods described herein are described in more detail in Section V.

In a further embodiment, a TAGE agent comprises a nucleic acid-guided endonuclease (e.g., RNA-guided endonuclease or DNA-guided endonuclease), such as Cas9, that recognizes a CRISPR sequence, and a CPP. Examples of CPPs that can be used in the compositions and methods described herein are described in more detail in Section V.

Proteins within the TAGE agent (i.e., at least a site-directed polypeptide and an extracellular cell membrane binding moiety) are stably associated such that the extracellular cell membrane binding moiety directs the site-directed modifying polypeptide to the cell surface and the site-directed modifying polypeptide is internalized into the target cell. In certain embodiments, the extracellular cell membrane binding moiety binds to the antigen on the cell surface such that the site-directed modifying polypeptide is internalized by the target cell but the extracellular cell membrane binding moiety (e.g., antigen binding protein, ligand, or CPP) is not internalized. In some embodiments, the site-directed modifying polypeptide and an extracellular cell membrane binding moiety are both internalized into the target cell.

Examples of an extracellular cell membrane binding moiety include, but are not limited to, an antigen binding polypeptide such as an antibody or fragment thereof, a ligand, or a CPP. In certain embodiments, a TAGE agent includes a two or more cell membrane binding agents, e.g., a CPP and an antibody, a CPP and a ligand, or a ligand and antibody. Such class pairings can, in certain embodiments, improve internalization of the site-directed modifying polypeptide. For example, in certain embodiments, a class pairing includes a TAGE agent comprising a CPP, an antigen binding polypeptide (e.g., an antibody), and a site-directed modifying polypeptide, in any arrangement. Other combinations of class pairings of cell binding moieties include a ligand, CPP, and a site-directed modifying polypeptide, in any arrangement. In one embodiment, a TAGE agent comprises an antibody, a peptide cell surface TCR, and a site-directed modifying polypeptide, in any arrangement.

In certain embodiments, when the site-directed modifying polypeptide is a nucleic acid-guided endonuclease, such as Cas9, the nucleic acid-guided endonuclease is associated with a guide nucleic acid to form a nucleoprotein. For example, the guide RNA (gRNA) binds to a RNA-guided nuclease to form a ribonucleoprotein (RNP) or a guide DNA binds to a DNA-guided nuclease to form a deoxyribonucleoprotein (DNP). In other embodiments, the nucleic acid-guided endonuclease is associated with a guide nucleic acid that comprises a DNA:RNA hybrid. In such instances, the ribonucleoprotein (i.e., the RNA-guided endonuclease and the guide RNA), deoxyribonucleoprotein (i.e., the DNA-guided endonuclease and the guide DNA), or the nucleic acid-guided endonuclease bound to a DNA:RNA hybrid guide are internalized into the target cell. In a separate embodiment, the guide nucleic acid (e.g., RNA, DNA, or DNA:RNA hybrid) is delivered to the target cell separately from the nucleic acid-guided endonuclease into the same cell. The guide nucleic acid (e.g., RNA, DNA, or DNA:RNA hybrid) may already be present in the target cell upon internalization of the nucleic acid-guided endonuclease upon contact with the TAGE agent.

A TAGE agent comprising a ligand or antigen binding protein specifically binds to an extracellular molecule (e.g., protein, glycan, lipid) localized on a target cell membrane. The target molecule can be, for example, an extracellular membrane-bound protein, but can also be a non-protein molecule such as a glycan or lipid. In one embodiment, the extracellular molecule is an extracellular protein that is expressed by the target cell, such as a ligand or a receptor. The extracellular target molecule may be associated with a specific disease condition or a specific tissue within in an organism. Examples of extracellular molecular targets associated with the cell membrane are described in the sections below.

The site-directed modifying polypeptide also comprises a conjugation moiety such that the extracellular cell membrane binding moiety can stably associate with the site-directed modifying polypeptide (thus forming a TAGE agent). The conjugation moiety provides for either a covalent or a non-covalent linkage between the extracellular cell membrane binding moiety and the site-directed modifying polypeptide.

In certain embodiments, the conjugation moiety useful for the present TAGE agents are stable extracellularly, prevent aggregation of TAGE molecules, and/or keep TAGE agents freely soluble in aqueous media and in a monomeric state. Before transport or delivery into a cell, the TAGE agent is stable and remains intact, e.g., the extracellular cell membrane binding moiety remains linked to the nucleic acid-guided endonuclease.

In one embodiment, the conjugation moiety is Protein A, wherein the site-directed modifying polypeptide comprises Protein A and the extracellular cell membrane binding moiety, e.g., an antigen binding protein, comprises an Fc region that can be bound by Protein A, e.g., an antibody comprising an Fc domain. In one embodiment, a site-directed modifying polypeptide comprises Protein A, or an Fc binding portion thereof.

In another embodiment, the conjugation moiety is a SpyCatcher/SpyTag peptide system. For example, in certain embodiments, the site-directed modifying polypeptide comprises SpyCatcher (e.g., at the N-terminus or C-terminus) and the extracellular cell membrane binding moiety comprises a SpyTag. For example, in instances where the site-directed modifying polypeptide comprises Cas9, the Cas9 may be conjugated to SpyCatcher to form SpyCatcher-Cas9 or Cas9-SpyCatcher. In one embodiment, the SpyTag peptide sequence is VPTIVMVDAYKRYK.

Other conjugation moieties useful in the TAGE agents provided herein include, but are not limited to, a Spycatcher tag, Snoop tag, Halo-tag (e.g., derived from haloalkane dehalogenase), Sortase, mono-avidin, ACP tag, a SNAP tag, or any other conjugation moieties known in the art. In one embodiment, the conjugation moiety is selected from Protein A, CBP, MBP, GST, poly(His), biotin/streptavidin, V5-tag, Myc-tag, HA-tag, NE-tag, His-tag, Flag tag, Halo-tag, Snap-tag, Fc-tag, Nus-tag, BCCP, thioredoxin, SnooprTag, SpyTag, SpyTag2, SpyCatcher, Isopeptag, SBP-tag, S-tag, AviTag, and calmodulin.

In some embodiments, the conjugation moiety is a chemical tag. For example, a chemical tag may be SNAP tag, a CLIP tag, a HaloTag or a TMP-tag. In one example, the chemical tag is a SNAP-tag or a CLIP-tag. SNAP and CLIP fusion proteins enable the specific, covalent attachment of virtually any molecule to a protein of interest. In another example, the chemical tag is a HaloTag. HaloTag involves a modular protein tagging system that allows different molecules to be linked onto a single genetic fusion, either in solution, in living cells, or in chemically fixed cells. In another example, the chemical tag is a TMP-tag.

In some embodiments, the conjugation moiety is an epitope tag. For example, an epitope tag may be a poly-histidine tag such as a hexahistidine tag or a dodecahistidine, a FLAG tag, a Myc tag, a HA tag, a GST tag or a V5 tag.

Depending on the conjugation approach, the site-directed modifying polypeptide and the extracellular cell membrane binding moiety may each be engineered to comprise complementary binding pairs that enable stable association upon contact. Exemplary binding moiety pairings include (i) streptavidin-binding peptide (streptavidin binding peptide; SBP) and streptavidin (STV), (ii) biotin and EMA (enhanced monomeric avidin), (iii) SpyTag (ST) and SpyCatcher (SC), (iv) Halo-tag and Halo-tag ligand, (v) and SNAP-Tag, (vi) Myc tag and anti-Myc immunoglobulins (vii) FLAG tag and anti-FLAG immunoglobulins, and (ix) ybbR tag and coenzyme A groups. In some embodiments, the conjugation moiety is selected from SBP, biotin, SpyTag, SpyCatcher, halo-tag, SNAP-tag, Myc tag, or FLAG tag.

In certain embodiments, the site-directed modifying polypeptide can alternatively be associated with an extracellular cell membrane binding moiety, e.g., an antigen binding protein, ligand, or CPP, via one or more linkers as described herein wherein the linker is a conjugation moiety.

The term “linker” as used herein means a divalent chemical moiety comprising a covalent bond or a chain of atoms that covalently attaches an extracellular cell membrane binding moiety to a site-directed modifying polypeptide to form a TAGE agent. Any known method of conjugation of peptides or macromolecules can be used in the context of the present disclosure. Generally, covalent attachment of the extracellular cell membrane binding moiety and the site-directed modifying polypeptide requires the linker to have two reactive functional groups, i.e., bivalency in a reactive sense. Bivalent linker reagents which are useful to attach two or more functional or biologically active moieties, such as peptides, nucleic acids, drugs, toxins, antibodies, haptens, and reporter groups are known, and methods for such conjugation have been described in, for example, Hermanson, G. T. (1996) Bioconjugate Techniques; Academic Press: New York, p 234-242, the disclosure of which is incorporated herein by reference as it pertains to linkers suitable for covalent conjugation. Further linkers are disclosed in, for example, Tsuchikama, K. and Zhiqiang, A. Protein and Cell, 9(1), p. 33-46, (2018), the disclosure of which is incorporated herein by reference as it pertains to linkers suitable for covalent conjugation.

Generally, linkers suitable for use in the compositions and methods disclosed are stable in circulation, but allow for release of the extracellular cell membrane binding moiety and/or the site-directed modifying polypeptide in the target cell or, alternatively, in close proximity to the target cell. Linkers suitable for the present disclosure may be broadly categorized as non-cleavable or cleavable, as well as intracellular or extracellular, each of which is further described herein below.

### Non-Cleavable Linkers

In some embodiments, the linker conjugating the extracellular cell membrane binding moiety and the site-directed modifying polypeptide is non-cleavable. Non-cleavable linkers comprise stable chemical bonds that are resistant to degradation (e.g., proteolysis). Generally, non-cleavable linkers require proteolytic degradation inside the target cell, and exhibit high extracellular stability. Non-cleavable linkers suitable for use herein further may include one or more groups selected from a bond, —(C═O)—, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted, and/or may include one or more heteroatoms (e.g., S, N, or O) in place of one or more carbon atoms. Non-limiting examples of such groups include alkylene (CH₂)ₚ, (C═O)(CH₂)ₚ, and polyethyleneglycol (PEG; (CH₂CH₂O)ₚ), units, wherein p is an integer from 1-6, independently selected for each occasion. Non-limiting examples of non-cleavable linker utilized in antibody-drug conjugation include those based on maleimidomethylcyclohexanecarboxylate, caproylmaleimide, and acetylphenylbutanoic acid.

### Cleavable Linkers

In some embodiments, the linker conjugating the extracellular cell membrane binding moiety and the site-directed modifying polypeptide is cleavable, such that cleavage of the linker (e.g., by a protease, such as metalloproteases) releases the CRISPR targeting element or the antibody or the antigen binding protein thereof, or both, from the TAGE agent in the intracellular or extracellular (e.g., upon binding of the molecule to the cell surface) environment. Cleavable linkers are designed to exploit the differences in local environments, e.g., extracellular and intracellular environments, for example, pH, reduction potential or enzyme concentration, to trigger the release of an TAGE agent component (i.e., extracellular cell membrane binding moiety (e.g., the antigen binding protein, ligand, or CPP), the site-directed modifying polypeptide, or both) in the target cell. Generally, cleavable linkers are relatively stable in circulation in vivo, but are particularly susceptible to cleavage in the intracellular environment through one or more mechanisms (e.g., including, but not limited to, activity of proteases, peptidases, and glucuronidases). Cleavable linkers used herein are stable outside the target cell and may be cleaved at some efficacious rate inside the target cell or in close proximity to the extracellular membrane of the target cell. An effective linker will: (i) maintain the specific binding properties of the extracellular cell membrane binding moiety, e.g., an antibody, ligand, or CPP; (ii) allow intra- or extracellular delivery of the TAGE agent or a component thereof (i.e., the site-directed modifying polypeptide); (iii) remain stable and intact, i.e. not cleaved, until the TAGE agent has been delivered or transported to its targeted site; and (iv) maintain the gene targeting effect (e.g., CRISPR) of the site-directed modifying polypeptide. Stability of the TAGE agent may be measured by standard analytical techniques such as mass spectroscopy, size determination by size exclusion chromatography or diffusion constant measurement by dynamic light scattering, HPLC, and the separation/analysis technique LC/MS.

Suitable cleavable linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a peptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome. Generally, linkers including such acid-labile functionalities tend to be relatively less stable extracellularly. This lower stability may be advantageous where extracellular cleavage is desired.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation. Disulfide-based linkers tend to be relatively unstable in circulation in plasma, however, this lower stability may be advantageous where extracellular cleavage is desired. Susceptibility to cleavage may also be tuned by e.g., introducing steric bulk near the disulfide moiety to hinder reductive cleavage.

Linkers susceptible to enzymatic hydrolysis can be, e.g., a peptide-containing linker that is cleaved by an intracellular peptidase or protease enzyme, including, but not limited to, a lysosomal or endosomal protease. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit. In certain embodiments, linkers comprising a peptide moiety may be susceptible to varying degrees of cleavage both intra- and extracellularly. Accordingly, in some embodiments, the linker comprises a dipeptide, and the TAGE agent is substantially cleaved extracellularly. Accordingly, in some embodiments, the linker comprises a dipeptide, and the TAGE agent is stable extracellularly and is cleaved intracellularly.

Linkers suitable for conjugating the extracellular cell membrane binding moiety (e.g., antigen binding protein, ligand, or CPP) as disclosed herein to a site-directed modifying polypeptide, as disclosed herein, include those capable of releasing the extracellular cell membrane binding moiety (e.g., antigen binding protein, ligand, or CPP) or the site-directed modifying polypeptide by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

Linkers suitable for use herein further may include one or more groups selected from C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted. Non-limiting examples of such groups include (CH₂)ₚ, (CH₂CH₂O)ₚ, and —(C═O)(CH₂)ₚ—units, wherein p is an integer from 1-6, independently selected for each occasion.

In some embodiments, the linker may include one or more of a hydrazine, a disulfide, a thioether, a dipeptide, a p-aminobenzyl (PAB) group, a heterocyclic self-immolative group, an optionally substituted C₁-C₆ alkyl, an optionally substituted C₁-C₆ heteroalkyl, an optionally substituted C₂-C₆ alkenyl, an optionally substituted C₂-C₆ heteroalkenyl, an optionally substituted C₂-C₆ alkynyl, an optionally substituted C₂-C₆ heteroalkynyl, an optionally substituted C₃—C₆ cycloalkyl, an optionally substituted heterocycloalkyl, an optionally substituted aryl, an optionally substituted heteroaryl, a solubility enhancing group, acyl, —(C═O)—, or —(CH₂CH₂O)ₚ— group, wherein p is an integer from 1-6. One of skill in the art will recognize that one or more of the groups listed may be present in the form of a bivalent (diradical) species, e.g., C₁-C₆ alkylene and the like.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB. In some embodiments, the linker comprises a combination of one or more of a peptide, oligosaccharide, —(CH₂)ₚ—, —(CH₂CH₂O)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

Suitable linkers may be substituted with groups which modulate solubility or reactivity. Suitable linkers may contain groups having solubility enhancing properties. Linkers including the (CH₂CH₂O)ₚ unit (polyethylene glycol, PEG), for example, can enhance solubility, as can alkyl chains substituted with amino, sulfonic acid, phosphonic acid or phosphoric acid residues. Linkers including such moieties are disclosed in, for example, U.S. Pat. Nos. 8,236,319 and 9,504,756, the disclosure of each of which is incorporated herein by reference as it pertains to linkers suitable for covalent conjugation. Linkers containing such groups are described, for example, in U.S. Pat. No. 9,636,421 and U.S. Patent Application Publication No. 2017/0298145, the disclosures of which are incorporated herein by reference as they pertain to linkers suitable for covalent conjugation.

Suitable linkers for covalently conjugating an extracellular cell membrane binding moiety and a site-directed modifying polypeptide as disclosed herein can have two reactive functional groups (i.e., two reactive termini), one for conjugation to the extracellular cell membrane binding moiety, and the other for conjugation to the site-directed modifying polypeptide. Suitable sites for conjugation on the extracellular cell membrane binding moiety are, in certain embodiments, nucleophilic, such as a thiol, amino group, or hydroxyl group. Reactive (e.g., nucleophilic) sites that may be present within an extracellular cell membrane binding moiety (e.g., antigen-binding protein, ligand, or CPP) as disclosed herein include, without limitation, nucleophilic substituents on amino acid residues such as (i) N-terminal amine groups, (ii) side chain amine groups, e.g. lysine, (iii) side chain thiol groups, e.g. cysteine, (iv) side chain hydroxyl groups, e.g. serine; or (iv) sugar hydroxyl or amino groups where the antibody is glycosylated. Suitable sites for conjugation on the extracellular cell membrane binding moiety include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. Accordingly, the antibody conjugation reactive terminus on the linker is, in certain embodiments, a thiol-reactive group such as a double bond (as in maleimide), a leaving group such as a chloro, bromo, iodo, or an R-sulfanyl group, or a carboxyl group.

Suitable sites for conjugation on the site-directed modifying polypeptide can also be, in certain embodiments, nucleophilic. Reactive (e.g., nucleophilic) sites that may be present within a site-directed modifying polypeptide as disclosed herein include, without limitation, nucleophilic substituents on amino acid residues such as (i) N-terminal amine groups, (ii) side chain amine groups, e.g. lysine, (iii) side chain thiol groups, e.g. cysteine, (iv) side chain hydroxyl groups, e.g. serine; or (iv) sugar hydroxyl or amino groups where the antibody is glycosylated. Suitable sites for conjugation on the site-directed modifying polypeptide include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. Accordingly, the site-directed modifying polypeptide conjugation reactive terminus on the linker is, in certain embodiments, a thiol-reactive group such as a double bond (as in maleimide), a leaving group such as a chloro, bromo, iodo, or an R-sulfanyl group, or a carboxyl group.

In some embodiments, the reactive functional group attached to the linker is a nucleophilic group which is reactive with an electrophilic group present on an extracellular cell membrane binding moiety (e.g., antigen binding protein, ligand, or CPP), the site-directed modifying polypeptide, or both. Useful electrophilic groups on an extracellular cell membrane binding moiety (e.g., antigen binding protein, ligand, or CPP) or site-directed modifying polypeptide include, but are not limited to, aldehyde and ketone carbonyl groups. The heteroatom of a nucleophilic group can react with an electrophilic group on an extracellular cell membrane binding moiety (e.g., antigen binding protein, ligand, or CPP) or site-directed modifying polypeptide and form a covalent bond to the extracellular cell membrane binding moiety (e.g., antigen binding protein, ligand, or CPP) or the site-directed modifying polypeptide. Useful nucleophilic groups include, but are not limited to, hydrazide, oxime, amino, hydroxyl, hydrazine, thiosemicarbazone, hydrazine carboxylate, and arylhydrazide.

In some embodiments, the TAGE agent as disclosed herein comprises a nucleoside or a nucleotide. Suitable sites for conjugation on such nucleosides or nucleotides include —OH or phosphate groups, respectively. Linkers and conjugation methods suitable for use in such embodiments are disclosed in, for example, Wang, T. P., et al., Bioconj. Chem. 21(9), 1642-55, 2010, and Bernardinelli, G. and Hogberg, B. Nucleic Acids Research, 45(18), p. e160; published online 16 August, 2017, the disclosure of each of which is incorporated herein by reference as it pertains to linkers suitable for covalent conjugation.

When the term “linker” is used in describing the linker in conjugated form, one or both of the reactive termini will be absent, (having been converted to a chemical moiety) or incomplete (such as being only the carbonyl of a carboxylic acid) because of the formation of the bonds between the linker and the extracellular cell membrane binding moiety, and/or between the linker and the site-directed modifying polypeptide. Accordingly, linkers useful herein include, without limitation, linkers containing a chemical moiety formed by a coupling reaction between a reactive functional group on the linker and a nucleophilic group or otherwise reactive substituent on the extracellular cell membrane binding moiety (e.g., antigen binding protein, ligand, or CPP), and a chemical moiety formed by a coupling reaction between a reactive functional group on the linker and a nucleophilic group on the site-directed modifying polypeptide.

Examples of chemical moieties formed by these coupling reactions result from reactions between chemically reactive functional groups, including a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive functional groups to form the chemical moiety include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. Suitable linkers may contain an electrophilic functional group for reaction with a nucleophilic functional group on the extracellular cell membrane binding moiety (e.g., antigen binding protein, ligand, or CPP), the site-directed modifying polypeptide, or both.

In some embodiments, the reactive functional group present within extracellular cell membrane binding moiety, the site-directed modifying polypeptide, or both as disclosed herein are amine or thiol moieties. Certain extracellular cell membrane binding moieties have reducible interchain disulfides, i.e. cysteine bridges. Extracellular cell membrane binding moieties may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into extracellular cell membrane binding moiety (e.g., antigen binding proteins, ligand, or CPP) through the reaction of lysines with 2-iminothiolane (Traut's reagent) resulting in conversion of an amine into a thiol. Reactive thiol groups may be introduced into the extracellular cell membrane binding moiety (antigen binding protein, ligand, or CPP) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Linkers suitable for the synthesis of the covalent conjugates as disclosed herein include, without limitation, reactive functional groups such as maleimide or a haloalkyl group. These groups may be present in linkers or cross linking reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, one or both of the reactive functional groups attached to the linker is a maleimide, azide, or alkyne. An example of a maleimide-containing linker is the non-cleavable maleimidocaproyl-based linker. Such linkers are described by Doronina et al., Bioconjugate Chem. 17:14-24, 2006, the disclosure of which is incorporated herein by reference as it pertains to linkers for chemical conjugation.

In some embodiments, the reactive functional group is —(C═O)— or —NH(C═O)—, such that the linker may be joined to the extracellular cell membrane binding moiety or the site-directed modifying polypeptide by an amide or urea moiety, respectively, resulting from reaction of the —(C═O)— or —NH(C═O)— group with an amino group of the extracellular cell membrane binding moiety or the site-directed modifying polypeptide, or both.

In some embodiments, the reactive functional group is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (hetero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis (sulfonylmethyl)methylcarbonyl group or elimination derivatives thereof, carbonyl halide group, or an allenamide group, each of which may be optionally substituted. In some embodiments, the reactive functional group comprises a cycloalkene group, a cycloalkyne group, or an optionally substituted (hetero)cycloalkynyl group.

Examples of suitable bivalent linker reagents suitable for preparing conjugates as disclosed herein include, but are not limited to, N-succinimidyl 4-(maleimidomethyl)cyclohexanecarboxylate (SMCC), N-succinimidyl-4-(N-maleimidomethyl)-cyclohexane-1-carboxy-(6-amidocaproate), which is a “long chain” analog of SMCC (LC-SMCC), κ-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(a-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI). Cross-linking reagents comprising a haloacetyl-based moiety include N-succinimidyl-4-(iodoacetyl)-aminobenzoate (SIAB), N-succinimidyl iodoacetate (SIA), N-succinimidyl bromoacetate (SBA), and N-succinimidyl 3-(bromoacetamido)propionate (SBAP).

It will be recognized by one of skill in the art that any one or more of the chemical groups, moieties and features disclosed herein may be combined in multiple ways to form linkers useful for conjugation of the extracellular cell membrane binding moiety as disclosed herein to a site-directed modifying polypeptide, as disclosed herein. Further linkers useful in conjunction with the compositions and methods described herein, are described, for example, in U.S. Patent Application Publication No. 2015/0218220, the disclosure of which is incorporated herein by reference as is pertain to linkers suitable for covalent conjugation.

### Site-Directed Modifying Polypeptide of the TAGE Agent

The TAGE agent comprises a site-directed modifying polypeptide, such as a nucleic acid-guided endonuclease (e.g., RNA-guided endonuclease (e.g., Cas9) or DNA-guided endonuclease) that recognizes a nucleic acid sequence in the target cell.

The site-directed modifying polypeptides used in the presently disclosed compositions and methods are site-specific, in that the polypeptide itself or an associated molecule recognizes and is targeted to a particular nucleic acid sequence or a set of similar sequences (i.e., target sequence(s)). In some embodiments, the site-directed modifying polypeptide (or its associated molecule) recognizes sequences that are similar in sequence, comprising conserved bases or motifs that can be degenerate at one or more positions.

In particular embodiments, the site-directed modifying polypeptide modifies the polynucleotide at particular location(s) (i.e., modification site(s)) outside of its target sequence. The modification site(s) modified by a particular site-directed modifying polypeptide are also generally specific to a particular sequence or set of similar sequences. In some of these embodiments, the site-directed modifying polypeptide modifies sequences that are similar in sequence, comprising conserved bases or motifs that can be degenerate at one or more positions. In other embodiments, the site-directed modifying polypeptide modifies sequences that are within a particular location relative to the target sequence(s). For example, the site-directed modifying polypeptide may modify sequences that are within a particular number of nucleic acids upstream or downstream from the target sequence(s).

As used herein with respect to site-directed modifying polypeptides, the term “modification” means any insertion, deletion, substitution, or chemical modification of at least one nucleotide the modification site or alternatively, a change in the expression of a gene that is adjacent to the target site. The substitution of at least one nucleotide in the modification site can be the result of the recruitment of a base editing domain, such as a cytidine deaminase or adenine deaminase domain (see, for example, Eid et al. (2018) Biochem J 475(11):1955-1964, which is herein incorporated in its entirety).

The change in expression of a gene adjacent to a target site can result from the recruitment of a transcriptional activation domain or transcriptional repression domain to the promoter region of the gene or the recruitment of an epigenetic modification domain that covalently modifies DNA or histone proteins to alter histone structure and/or chromosomal structure without altering the DNA sequence, leading to changes in gene expression of an adjacent gene. The term “modification” also encompasses the recruitment to a target site of a detectable label that can be conjugated to the site-directed modifying polypeptide or an associated molecule (e.g., gRNA) that allows for the detection of a specific nucleic acid sequence (e.g., a disease-associated sequence).

In some embodiments, the site-directed modifying polypeptide is a nuclease or variant thereof and the agent comprising the nuclease or variant thereof is thus referred to herein as a gene editing cell targeting (TAGE) agent. As used herein a “nuclease” refers to an enzyme which cleaves a phosphodiester bond in the backbone of a polynucleotide chain. Suitable nucleases for the presently disclosed compositions and methods can have endonuclease and/or exonuclease activity. An exonuclease cleaves nucleotides one at a time from the end of a polynucleotide chain. An endonuclease cleaves a polynucleotide chain by cleaving phosphodiester bonds within a polynucleotide chain, other than those at the two ends of a polynucleotide chain. The nuclease can cleave RNA polynucleotide chains (i.e., ribonuclease) and/or DNA polynucleotide chains (i.e., deoxyribonuclease).

Nucleases cleave polynucleotide chains, resulting in a cleavage site. As used herein, the term “cleave” refers to the hydrolysis of phosphodiester bonds within the backbone of a polynucleotide chain. Cleavage by nucleases of the presently disclosed TAGE agents can be single-stranded or double-stranded. In some embodiments, a double-stranded cleavage of DNA is achieved via cleavage with two nucleases wherein each nuclease cleaves a single strand of the DNA. Cleavage by the nuclease can result in blunt ends or staggered ends.

Non-limiting examples of nucleases suitable for the presently disclosed compositions and methods include meganucleases, such as homing endonucleases; restriction endonucleases, such as Type IIS endonucleases (e.g., Fokl)); zinc finger nucleases; transcription activator-like effector nucleases (TALENs), and nucleic acid-guided nucleases (e.g., RNA-guided endonuclease, DNA-guided endonuclease, or DNA/RNA-guided endonuclease).

As used herein, a “meganuclease” refers to an endonuclease that binds DNA at a target sequence that is greater than 12 base pairs in length. Meganucleases bind to double-stranded DNA as heterodimers. Suitable meganucleases for the presently disclosed compositions and methods include homing endonucleases, such as those of the LAGLIDADG (SEQ ID NO: 1) family comprising this amino acid motif or a variant thereof.

As used herein, a “zinc finger nuclease” or “ZFN” refers to a chimeric protein comprising a zinc finger DNA-binding domain fused to a nuclease domain from an exonuclease or endonuclease, such as a restriction endonuclease or meganuclease. The zinc finger DNA-binding domain is bound by a zinc ion that serves to stabilize the unique structure.

As used herein, a “transcription activator-like effector nuclease” or “TALEN” refers to a chimeric protein comprising a DNA-binding domain comprising multiple TAL domain repeats fused to a nuclease domain from an exonuclease or endonuclease, such as a restriction endonuclease or meganuclease. TAL domain repeats can be derived from the TALE family of proteins from the Xanthomonas genus of Proteobacteria. TAL domain repeats are 33-34 amino acid sequences with hypervariable 12^{th} and 13^{th} amino acids that are referred to as the repeat variable diresidue (RVD). The RVD imparts specificity of target sequence binding. The TAL domain repeats can be engineered through rational or experimental means to produce variant TALENs that have a specific target sequence specificity (see, for example, Boch et al. (2009) Science 326(5959):1509-1512 and Moscou and Bogdanove (2009) Science 326(5959):1501, each of which is incorporated by reference in its entirety). DNA cleavage by a TALEN requires two DNA target sequences flanking a nonspecific spacer region, wherein each DNA target sequence is bound by a TALEN monomer. In some embodiments, the TALEN comprises a compact TALEN, which refers to an endonuclease comprising a DNA-binding domain with one or more TAL domain repeats fused in any orientation to any portion of a homing endonuclease (e.g., I-Tevl, Mmel, EndA, End1, I-Basl, I-TevIl, I-TevIII, I-Twol, Mspl, Mval, NucA, and NucM). Compact TALENs are advantageous in that they do not require dimerization for DNA processing activity, thus only requiring a single target site.

As used herein, a “nucleic acid-guided nuclease” refers to a nuclease that is directed to a specific target sequence based on the complementarity (full or partial) between a guide nucleic acid (i.e., guide RNA or gRNA, guide DNA or gDNA, or guide DNA/RNA hybrid) that is associated with the nuclease and a target sequence. The binding between the guide RNA and the target sequence serves to recruit the nuclease to the vicinity of the target sequence. Non-limiting examples of nucleic acid-guided nucleases suitable for the presently disclosed compositions and methods include naturally-occurring Clustered Regularly Interspaced Short Palindromic Repeats (CRISPR)-associated (Cas) polypeptides from a prokaryotic organism (e.g., bacteria, archaea) or variants thereof. CRISPR sequences found within prokaryotic organisms are sequences that are derived from fragments of polynucleotides from invading viruses and are used to recognize similar viruses during subsequent infections and cleave viral polynucleotides via CRISPR-associated (Cas) polypeptides that function as an RNA-guided nuclease to cleave the viral polynucleotides. As used herein, a “CRISPR-associated polypeptide” or “Cas polypeptide” refers to a naturally-occurring polypeptide that is found within proximity to CRISPR sequences within a naturally-occurring CRISPR system. Certain Cas polypeptides function as RNA-guided nucleases.

There are at least two classes of naturally-occurring CRISPR systems, Class 1 and Class 2. In general, the nucleic acid-guided nucleases of the presently disclosed compositions and methods are Class 2 Cas polypeptides or variants thereof given that the Class 2 CRISPR systems comprise a single polypeptide with nucleic acid-guided nuclease activity, whereas Class 1 CRISPR systems require a complex of proteins for nuclease activity. There are at least three known types of Class 2 CRISPR systems, Type II, Type V, and Type VI, among which there are multiple subtypes (subtype II-A, II-B, II-C, V-A, V-B, V-C, VI-A, VI-B, and VI-C, among other undefined or putative subtypes). In general, Type II and Type V-B systems require a tracrRNA, in addition to crRNA, for activity. In contrast, Type V-A and Type VI only require a crRNA for activity. All known Type II and Type V RNA-guided nucleases target double-stranded DNA, whereas all known Type VI RNA-guided nucleases target single-stranded RNA. The RNA-guided nucleases of Type II CRISPR systems are referred to as Cas9 herein and in the literature. In some embodiments, the nucleic acid-guided nuclease of the presently disclosed compositions and methods is a Type II Cas9 protein or a variant thereof. Type V Cas polypeptides that function as RNA-guided nucleases do not require tracrRNA for targeting and cleavage of target sequences. The RNA-guided nuclease of Type VA CRISPR systems are referred to as Cpf1; of Type VB CRISPR systems are referred to as C2C1; of Type VC CRISPR systems are referred to as Cas12C or C2C3; of Type VIA CRISPR systems are referred to as C2C2 or Cas13A1; of Type VIB CRISPR systems are referred to as Cas13B; and of Type VIC CRISPR systems are referred to as Cas13A2 herein and in the literature. In certain embodiments, the nucleic acid-guided nuclease of the presently disclosed compositions and methods is a Type VA Cpf1 protein or a variant thereof. Naturally-occurring Cas polypeptides and variants thereof that function as nucleic acid-guided nucleases are known in the art and include, but are not limited to Streptococcus pyogenes Cas9, Staphylococcus aureus Cas9, Streptococcus thermophilus Cas9, Francisella novicida Cpf1, or those described in Shmakov et al. (2017) Nat Rev Microbiol 15(3):169-182; Makarova et al. (2015) Nat Rev Microbiol 13(11):722-736; and U.S. Pat. No. 9,790,490, each of which is incorporated herein in its entirety. Class 2 Type V CRISPR nucleases include Cas12 and any subtypes of Cas12, such as Cas12a, Cas12b, Cas12c, Cas12d, Cas12e, Cas12f, Cas12g, Cas12h, and Cas12i. Class 2 Type VI CRISPR nucleases including Cas13 can be included in the TAGE agent in order to cleave RNA target sequences.

The nucleic acid-guided nuclease of the presently disclosed compositions and methods can be a naturally-occurring nucleic acid-guided nuclease (e.g., S. pyogenes Cas9) or a variant thereof. Variant nucleic acid-guided nucleases can be engineered or naturally occurring variants that contain substitutions, deletions, or additions of amino acids that, for example, alter the activity of one or more of the nuclease domains, fuse the nucleic acid-guided nuclease to a heterologous domain that imparts a modifying property (e.g., transcriptional activation domain, epigenetic modification domain, detectable label), modify the stability of the nuclease, or modify the specificity of the nuclease.

In some embodiments, a nucleic acid-guided nuclease includes one or more mutations to improve specificity for a target site and/or stability in the intracellular microenvironment. For example, where the protein is Cas9 (e.g., SpCas9) or a modified Cas9, it may be beneficial to delete any or all residues from N175 to R307 (inclusive) of the Rec2 domain. It may be found that a smaller, or lower-molecular mass, version of the nuclease is more effective. In some embodiments, the nuclease comprises at least one substitution relative to a naturally-occurring version of the nuclease. For example, where the protein is Cas9 or a modified Cas9, it may be beneficial to mutate C80 or C574 (or homologs thereof, in modified proteins with indels). In Cas9, desirable substitutions may include any of C80A, C80L, C80I, C80V, C80K, C574E, C574D, C574N, C574Q (in any combination) and in particular C80A. Substitutions may be included to reduce intracellular protein binding of the nuclease and/or increase target site specificity. Additionally or alternatively, substitutions may be included to reduce off-target toxicity of the composition.

The nucleic acid-guided nuclease is directed to a particular target sequence through its association with a guide nucleic acid (e.g., guide RNA (gRNA), guide DNA (gDNA)). The nucleic acid-guided nuclease is bound to the guide nucleic acid via non-covalent interactions, thus forming a complex. The polynucleotide-targeting nucleic acid provides target specificity to the complex by comprising a nucleotide sequence that is complementary to a sequence of a target sequence. The nucleic acid-guided nuclease of the complex or a domain or label fused or otherwise conjugated thereto provides the site-specific activity. In other words, the nucleic acid-guided nuclease is guided to a target polynucleotide sequence (e.g. a target sequence in a chromosomal nucleic acid; a target sequence in an extrachromosomal nucleic acid, e.g. an episomal nucleic acid, a minicircle; a target sequence in a mitochondrial nucleic acid; a target sequence in a chloroplast nucleic acid; a target sequence in a plasmid) by virtue of its association with the protein-binding segment of the polynucleotide-targeting guide nucleic acid.

Thus, the guide nucleic acid comprises two segments, a “polynucleotide-targeting segment” and a “polypeptide-binding segment.” By “segment” it is meant a segment/section/region of a molecule (e.g., a contiguous stretch of nucleotides in an RNA). A segment can also refer to a region/section of a complex such that a segment may comprise regions of more than one molecule. For example, in some cases the polypeptide-binding segment (described below) of a polynucleotide-targeting nucleic acid comprises only one nucleic acid molecule and the polypeptide-binding segment therefore comprises a region of that nucleic acid molecule. In other cases, the polypeptide-binding segment (described below) of a DNA-targeting nucleic acid comprises two separate molecules that are hybridized along a region of complementarity.

The polynucleotide-targeting segment (or “polynucleotide-targeting sequence” or “guide sequence”) comprises a nucleotide sequence that is complementary (fully or partially) to a specific sequence within a target sequence (for example, the complementary strand of a target DNA sequence). The polypeptide-binding segment (or “polypeptide-binding sequence”) interacts with a nucleic acid-guided nuclease. In general, site-specific cleavage or modification of the target DNA by a nucleic acid-guided nuclease occurs at locations determined by both (i) base-pairing complementarity between the polynucleotide-targeting sequence of the nucleic acid and the target DNA; and (ii) a short motif (referred to as the protospacer adjacent motif (PAM)) in the target DNA.

A protospacer adjacent motif can be of different lengths and can be a variable distance from the target sequence, although the PAM is generally within about 1 to about 10 nucleotides from the target sequence, including about 1, about 2, about 3, about 4, about 5, about 6, about 7, about 8, about 9, or about 10 nucleotides from the target sequence. The PAM can be 5′ or 3′ of the target sequence. Generally, the PAM is a consensus sequence of about 3-4 nucleotides, but in particular embodiments, can be 2, 3, 4, 5, 6, 7, 8, 9, or more nucleotides in length. Methods for identifying a preferred PAM sequence or consensus sequence for a given RNA-guided nuclease are known in the art and include, but are not limited to the PAM depletion assay described by Karvelis et al. (2015) Genome Biol 16:253, or the assay disclosed in Pattanayak et al. (2013) Nat Biotechnol 31(9):839-43, each of which is incorporated by reference in its entirety.

The polynucleotide-targeting sequence (i.e., guide sequence) is the nucleotide sequence that directly hybridizes with the target sequence of interest. The guide sequence is engineered to be fully or partially complementary with the target sequence of interest. In various embodiments, the guide sequence can comprise from about 8 nucleotides to about 30 nucleotides, or more. For example, the guide sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the guide sequence is about 10 to about 26 nucleotides in length, or about 12 to about 30 nucleotides in length. In particular embodiments, the guide sequence is about 30 nucleotides in length. In some embodiments, the degree of complementarity between a guide sequence and its corresponding target sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the guide sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24).

In some embodiments, a guide nucleic acid comprises two separate nucleic acid molecules (an “activator-nucleic acid” and a “targeter-nucleic acid”, see below) and is referred to herein as a “double-molecule guide nucleic acid” or a “two-molecule guide nucleic acid.” In other embodiments, the subject guide nucleic acid is a single nucleic acid molecule (single polynucleotide) and is referred to herein as a “single-molecule guide nucleic acid,” a “single-guide nucleic acid,” or an “sgNA.” The term “guide nucleic acid” or “gNA” is inclusive, referring both to double-molecule guide nucleic acids and to single-molecule guide nucleic acids (i.e., sgNAs). In those embodiments wherein the guide nucleic acid is an RNA, the gRNA can be a double-molecule guide RNA or a single-guide RNA. Likewise, in those embodiments wherein the guide nucleic acid is a DNA, the gDNA can be a double-molecule guide DNA or a single-guide DNA.

An exemplary two-molecule guide nucleic acid comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA-like molecule (targeter-RNA) comprises both the polynucleotide-targeting segment (single stranded) of the guide RNA and a stretch (“duplex-forming segment”) of nucleotides that forms one half of the dsRNA duplex of the polypeptide-binding segment of the guide RNA, also referred to herein as the CRISPR repeat sequence.

The term “activator-nucleic acid” or “activator-NA” is used herein to mean a tracrRNA-like molecule of a double-molecule guide nucleic acid. The term “targeter-nucleic acid” or “targeter-NA” is used herein to mean a crRNA-like molecule of a double-molecule guide nucleic acid. The term “duplex-forming segment” is used herein to mean the stretch of nucleotides of an activator-NA or a targeter-NA that contributes to the formation of the dsRNA duplex by hybridizing to a stretch of nucleotides of a corresponding activator-NA or targeter-NA molecule. In other words, an activator-NA comprises a duplex-forming segment that is complementary to the duplex-forming segment of the corresponding targeter-NA. As such, an activator-NA comprises a duplex-forming segment while a targeter-NA comprises both a duplex-forming segment and the DNA-targeting segment of the guide nucleic acid. Therefore, a subject double-molecule guide nucleic acid can be comprised of any corresponding activator-NA and targeter-NA pair.

The activator-NA comprises a CRISPR repeat sequence comprising a nucleotide sequence that comprises a region with sufficient complementarity to hybridize to an activator-NA (the other part of the polypeptide-binding segment of the guide nucleic acid). In various embodiments, the CRISPR repeat sequence can comprise from about 8 nucleotides to about 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and the antirepeat region of its corresponding tracr sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

A corresponding tracrRNA-like molecule (i.e., activator-NA) comprises a stretch of nucleotides (duplex-forming segment) that forms the other part of the double-stranded duplex of the polypeptide-binding segment of the guide nucleic acid. In other words, a stretch of nucleotides of a crRNA-like molecule (i.e., the CRISPR repeat sequence) are complementary to and hybridize with a stretch of nucleotides of a tracrRNA-like molecule (i.e., the anti-repeat sequence) to form the double-stranded duplex of the polypeptide-binding domain of the guide nucleic acid. The crRNA-like molecule additionally provides the single stranded DNA-targeting segment. Thus, a crRNA-like and a tracrRNA-like molecule (as a corresponding pair) hybridize to form a guide nucleic acid. The exact sequence of a given crRNA or tracrRNA molecule is characteristic of the CRISPR system and species in which the RNA molecules are found. A subject double-molecule guide RNA can comprise any corresponding crRNA and tracrRNA pair.

A trans-activating-like CRISPR RNA or tracrRNA-like molecule (also referred to herein as an “activator-NA”) comprises a nucleotide sequence comprising a region that has sufficient complementarity to hybridize to a CRISPR repeat sequence of a crRNA, which is referred to herein as the anti-repeat region. In some embodiments, the tracrRNA-like molecule further comprises a region with secondary structure (e.g., stem-loop) or forms secondary structure upon hybridizing with its corresponding crRNA. In particular embodiments, the region of the tracrRNA-like molecule that is fully or partially complementary to a CRISPR repeat sequence is at the 5′ end of the molecule and the 3′ end of the tracrRNA-like molecule comprises secondary structure. This region of secondary structure generally comprises several hairpin structures, including the nexus hairpin, which is found adjacent to the anti-repeat sequence. The nexus hairpin often has a conserved nucleotide sequence in the base of the hairpin stem, with the motif UNANNC found in many nexus hairpins in tracrRNAs. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In various embodiments, the anti-repeat region of the tracrRNA-like molecule that is fully or partially complementary to the CRISPR repeat sequence comprises from about 8 nucleotides to about 30 nucleotides, or more. For example, the region of base pairing between the tracrRNA-like anti-repeat sequence and the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and its corresponding tracrRNA-like anti-repeat sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In various embodiments, the entire tracrRNA-like molecule can comprise from about 60 nucleotides to more than about 140 nucleotides. For example, the tracrRNA-like molecule can be about 60, about 65, about 70, about 75, about 80, about 85, about 90, about 95, about 100, about 105, about 110, about 115, about 120, about 125, about 130, about 135, about 140, or more nucleotides in length. In particular embodiments, the tracrRNA-like molecule is about 80 to about 100 nucleotides in length, including about 80, about 81, about 82, about 83, about 84, about 85, about 86, about 87, about 88, about 89, about 90, about 91, about 92, about 93, about 94, about 95, about 96, about 97, about 98, about 99, and about 100 nucleotides in length.

A subject single-molecule guide nucleic acid (i.e., sgNA) comprises two stretches of nucleotides (a targeter-NA and an activator-NA) that are complementary to one another, are covalently linked by intervening nucleotides (“linkers” or “linker nucleotides”), and hybridize to form the double stranded nucleic acid duplex of the protein-binding segment, thus resulting in a stem-loop structure. The targeter-NA and the activator-NA can be covalently linked via the 3′ end of the targeter-NA and the 5′ end of the activator-NA. Alternatively, the targeter-NA and the activator-NA can be covalently linked via the 5′ end of the targeter-NA and the 3′ end of the activator-NA.

The linker of a single-molecule DNA-targeting nucleic acid can have a length of from about 3 nucleotides to about 100 nucleotides. For example, the linker can have a length of from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt or from about 3 nt to about 10 nt, including but not limited to about 3, about 4, about 5, about 6, about 7, about 8, about 9, about 10, about 11, about 12, about 13, about 14, about 15, about 16, about 17, about 18, about 19, about 20, or more nucleotides. In some embodiments, the linker of a single-molecule DNA-targeting nucleic acid is 4 nt.

An exemplary single-molecule DNA-targeting nucleic acid comprises two complementary stretches of nucleotides that hybridize to form a double-stranded duplex, along with a guide sequence that hybridizes to a specific target sequence.

Appropriate naturally-occurring cognate pairs of crRNAs (and, in some embodiments, tracrRNAs) are known for most Cas proteins that function as nucleic acid-guided nucleases that have been discovered or can be determined for a specific naturally-occurring Cas protein that has nucleic acid-guided nuclease activity by sequencing and analyzing flanking sequences of the Cas nucleic acid-guided nuclease protein to identify tracrRNA-coding sequence, and thus, the tracrRNA sequence, by searching for known antirepeat-coding sequences or a variant thereof. Antirepeat regions of the tracrRNA comprise one-half of the ds protein-binding duplex. The complementary repeat sequence that comprises one-half of the ds protein-binding duplex is called the CRISPR repeat. CRISPR repeat and antirepeat sequences utilized by known CRISPR nucleic acid-guided nucleases are known in the art and can be found, for example, at the CRISPR database on the world wide web at crispr.i2bc.paris-saclay.fr/crispr/.

The single guide nucleic acid or dual-guide nucleic acid can be synthesized chemically or via in vitro transcription. Assays for determining sequence-specific binding between a nucleic acid-guided nuclease and a guide nucleic acid are known in the art and include, but are not limited to, in vitro binding assays between an expressed nucleic acid-guided nuclease and the guide nucleic acid, which can be tagged with a detectable label (e.g., biotin) and used in a pull-down detection assay in which the nucleoprotein complex is captured via the detectable label (e.g., with streptavidin beads). A control guide nucleic acid with an unrelated sequence or structure to the guide nucleic acid can be used as a negative control for non-specific binding of the nucleic acid-guided nuclease to nucleic acids.

In some embodiments, the DNA-targeting RNA, gRNA, or sgRNA or nucleotide sequence encoding the DNA-targeting RNA, gRNA, or sgRNA comprises modifications of the nucleotide sequence. In some cases, the sgRNA (e.g., truncated sgRNA) comprises a first nucleotide sequence that is complementary to the target nucleic acid and a second nucleotide sequence that interacts with a Cas polypeptide. In other instances, the sgRNA comprises one or more modified nucleotides. In some cases, one or more of the nucleotides in the first nucleotide sequence and/or the second nucleotide sequence are modified nucleotides.

In some embodiments, the modified nucleotides comprise a modification in a ribose group, a phosphate group, a nucleobase, or a combination thereof. In some instances, the modification in the ribose group comprises a modification at the 2′ position of the ribose group. In some cases, the modification at the 2′ position of the ribose group is selected from the group consisting of 2′-O-methyl, 2′-fluoro, 2′-deoxy, 2′-O-(2-methoxyethyl), and a combination thereof. In other instances, the modification in the phosphate group comprises a phosphorothioate modification. In other embodiments, the modified nucleotides are selected from the group consisting of a 2′-ribo 3′-phosphorothioate (S), 2′-O-methyl (M) nucleotide, a 2′-O-methyl 3′-phosphorothioate (MS) nucleotide, a 2′-O-methyl 3′-thioPACE (MSP) nucleotide, and a combination thereof.

In certain embodiments, the site-directed modifying polypeptide of the presently disclosed compositions and methods comprise a nuclease variant that functions as a nickase, wherein the nuclease comprises a mutation in comparison to the wild-type nuclease that results in the nuclease only being capable of cleaving a single strand of a double-stranded nucleic acid molecule, or lacks nuclease activity altogether (i.e., nuclease-dead).

A nuclease, such as a nucleic acid-guided nuclease, that functions as a nickase only comprises a single functioning nuclease domain. In some of these embodiments, additional nuclease domains have been mutated such that the nuclease activity of that particular domain is reduced or eliminated.

In other embodiments, the nuclease (e.g., RNA-guided nuclease) lacks nuclease activity completely and is referred to herein as nuclease-dead. In some of these embodiments, all nuclease domains within the nuclease have been mutated such that all nuclease activity of the polypeptide has been eliminated. Any method known in the art can be used to introduce mutations into one or more nuclease domains of a site-directed nuclease, including those set forth in U.S. Publ. Nos. 2014/0068797 and U.S. Pat. No. 9,790,490, each of which is incorporated by reference in its entirety.

Any mutation within a nuclease domain that reduces or eliminates the nuclease activity can be used to generate a nucleic acid-guided nuclease having nickase activity or a nuclease-dead nucleic acid-guided nuclease. Such mutations are known in the art and include, but are not limited to the D10A mutation within the RuvC domain or H840A mutation within the HNH domain of the S. pyogenes Cas9 or at similar position(s) within another nucleic acid-guided nuclease when aligned for maximal homology with the S. pyogenes Cas9. Other positions within the nuclease domains of S. pyogenes Cas9 that can be mutated to generate a nickase or nuclease-dead protein include G12, G17, E762, N854, N863, H982, H983, and D986. Other mutations within a nuclease domain of a nucleic acid-guided nuclease that can lead to nickase or nuclease-dead proteins include a D917A, E1006A, E1028A, D1227A, D1255A, N1257A, D917A, E1006A, E1028A, D1227A, D1255A, and N1257A of the Francisella novicida Cpf1 protein or at similar position(s) within another nucleic acid-guided nuclease when aligned for maximal homology with the F. novicida Cpf1 protein (U.S. Pat. No. 9,790,490, which is incorporated by reference in its entirety).

Site-directed modifying polypeptides comprising a nuclease-dead domain can further comprise a domain capable of modifying a polynucleotide. Non-limiting examples of modifying domains that may be fused to a nuclease-dead domain include but are not limited to, a transcriptional activation or repression domain, a base editing domain, and an epigenetic modification domain. In other embodiments, the site-directed modifying polypeptide comprising a nuclease-dead domain further comprises a detectable label that can aid in detecting the presence of the target sequence.

The epigenetic modification domain that can be fused to a nuclease-dead domain serves to covalently modify DNA or histone proteins to alter histone structure and/or chromosomal structure without altering the DNA sequence itself, leading to changes in gene expression (upregulation or downregulation). Non-limiting examples of epigenetic modifications that can be induced by site-directed modifying polypeptides include the following alterations in histone residues and the reverse reactions thereof: sumoylation, methylation of arginine or lysine residues, acetylation or ubiquitination of lysine residues, phosphorylation of serine and/or threonine residues; and the following alterations of DNA and the reverse reactions thereof: methylation or hydroxymethylation of cytosine residues. Non-limiting examples of epigenetic modification domains thus include histone acetyltransferase domains, histone deacetylation domains, histone methyltransferase domains, histone demethylase domains, DNA methyltransferase domains, and DNA demethylase domains.

In some embodiments, the site-directed polypeptide comprises a transcriptional activation domain that activates the transcription of at least one adjacent gene through the interaction with transcriptional control elements and/or transcriptional regulatory proteins, such as transcription factors or RNA polymerases. Suitable transcriptional activation domains are known in the art and include, but are not limited to, VP16 activation domains.

In other embodiments, the site-directed polypeptide comprises a transcriptional repressor domain, which can also interact with transcriptional control elements and/or transcriptional regulatory proteins, such as transcription factors or RNA polymerases, to reduce or terminate transcription of at least one adjacent gene. Suitable transcriptional repression domains are known in the art and include, but are not limited to, IKB and KRAB domains.

In still other embodiments, the site-directed modifying polypeptide comprising a nuclease-dead domain further comprises a detectable label that can aid in detecting the presence of the target sequence, which may be a disease-associated sequence. A detectable label is a molecule that can be visualized or otherwise observed. The detectable label may be fused to the nucleic-acid guided nuclease as a fusion protein (e.g., fluorescent protein) or may be a small molecule conjugated to the nuclease polypeptide that can be detected visually or by other means. Detectable labels that can be fused to the presently disclosed nucleic-acid guided nucleases as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as ³H and ³⁵S.

The nucleic acid-guided nuclease can be delivered as part of a TAGE agent into a cell as a nucleoprotein complex comprising the nucleic acid-guided nuclease bound to its guide nucleic acid. Alternatively, the nucleic acid-guided nuclease is delivered as a TAGE agent and the guide nucleic acid is provided separately. In certain embodiments, a guide RNA can be introduced into a target cell as an RNA molecule. The guide RNA can be transcribed in vitro or chemically synthesized. In other embodiments, a nucleotide sequence encoding the guide RNA is introduced into the cell. In some of these embodiments, the nucleotide sequence encoding the guide RNA is operably linked to a promoter (e.g., an RNA polymerase III promoter), which can be a native promoter or heterologous to the guide RNA-encoding nucleotide sequence.

In certain embodiments, the site-directed polypeptide can comprise additional amino acid sequences, such as at least one nuclear localization sequence (NLS). Nuclear localization sequences enhance transport of the site-directed polypeptide into the nucleus of a cell. Proteins that are imported into the nucleus bind to one or more of the proteins within the nuclear pore complex, such as importin/karypherin proteins, which generally bind best to lysine and arginine residues. The best characterized pathway for nuclear localization involves short peptide sequence which binds to the importin-α protein. These nuclear localization sequences often comprise stretches of basic amino acids and given that there are two such binding sites on importin-α, two basic sequences separated by at least 10 amino acids can make up a bipartite NLS. The second most characterized pathway of nuclear import involves proteins that bind to the importin-β1 protein, such as the HIV-TAT and HIV-REV proteins, which use the sequences RKKRRQRRR (SEQ ID NO: 2) and RQARRNRRRRWR (SEQ ID NO: 3), respectively to bind to importin-β1. Other nuclear localization sequences are known in the art (see, e.g., Lange et al., J. Biol. Chem. (2007) 282:5101-5105). The NLS can be the naturally-occurring NLS of the site-directed polypeptide or a heterologous NLS. As used herein, “heterologous” in reference to a sequence is a sequence that originates from a foreign species, or, if from the same species, is substantially modified from its native form in composition and/or genomic locus by deliberate human intervention. Non-limiting examples of NLS sequences that can be used to enhance the nuclear localization of the site-directed polypeptides include the NLS of the SV40 Large T-antigen and c-Myc. In certain embodiments, the NLS comprises the amino acid sequence PKKKRKV (SEQ ID NO: 4).

The site-directed polypeptide can comprise more than one NLS, such as two, three, four, five, six, or more NLS sequences. Each of the multiple NLSs can be unique in sequence or there can be more than one of the same NLS sequence used. The NLS can be on the amino-terminal (N-terminal) end of the site-directed polypeptide, the carboxy-terminal (C-terminal) end, or both the N-terminal and C-terminal ends of the polypeptide. In certain embodiments, the site-directed polypeptide comprises four NLS sequences on its N-terminal end. In other embodiments, the site-directed polypeptide comprises two NLS sequences on the C-terminal end of the site-directed polypeptide. In still other embodiments, the site-directed polypeptide comprises four NLS sequences on its N-terminal end and two NLS sequences on its C-terminal end.

In certain embodiments, the site-directed polypeptide comprises a cell penetrating peptide (CPP), which induces the absorption of a linked protein or peptide through the plasma membrane of a cell. Generally, CPPs induce entry into the cell because of their general shape and tendency to either self-assemble into a membrane-spanning pore, or to have several positively charged residues, which interact with the negatively charged phospholipid outer membrane inducing curvature of the membrane, which in turn activates internalization. Exemplary permeable peptides include, but are not limited to, transportan, PEP1, MPG, p-VEC, MAP, CADY, polyR, HIV-TAT, HIV-REV, Penetratin, R6W3, P22N, DPV3, DPV6, K-FGF, and C105Y, and are reviewed in van den Berg and Dowdy (2011) Current Opinion in Biotechnology 22:888-893 and Farkhani et al. (2014) Peptides 57:78-94, each of which is herein incorporated by reference in its entirety.

Along with or as an alternative to an NLS, the site-directed polypeptide can comprise additional heterologous amino acid sequences, such as a detectable label (e.g., fluorescent protein) described elsewhere herein, or a purification tag, to form a fusion protein. A purification tag is any molecule that can be utilized to isolate a protein or fused protein from a mixture (e.g., biological sample, culture medium). Non-limiting examples of purification tags include biotin, myc, maltose binding protein (MBP), and glutathione-S-transferase (GST).

The presently disclosed compositions and methods can be used to edit genomes through the introduction of a sequence-specific, double-stranded break that is repaired (via e.g., error-prone non-homologous end-joining (NHEJ), microhomology-mediated end joining (MMEJ), or alternative end-joining (alt-EJ) pathway) to introduce a mutation at a specific genomic location. Due to the error-prone nature of repair processes, repair of the double-stranded break can result in a modification to the target sequence. Alternatively, a donor template polynucleotide may be integrated into or exchanged with the target sequence during the course of repair of the introduced double-stranded break, resulting in the introduction of the exogenous donor sequence. Accordingly, the compositions and methods can further comprise a donor template polynucleotide that may comprise flanking homologous ends. In some of these embodiments, the donor template polynucleotide is tethered to the TAGE agent via a linker as described elsewhere herein (e.g., the donor template polynucleotide is bound to the site-directed polypeptide via a cleavable linker).

In some embodiments, the donor sequence alters the original target sequence such that the newly integrated donor sequence will not be recognized and cleaved by the nucleic acid-guided nuclease. The donor sequence may comprise flanking sequences that have substantial sequence identity with the sequences flanking the target sequence to enhance the integration of the donor sequence via homology-directed repair. In particular embodiments wherein the nucleic acid-guided nuclease generates double-stranded staggered breaks, the donor polynucleotide can be flanked by compatible overhangs, allowing for incorporation of the donor sequence via a non-homologous repair process during repair of the double-stranded break.

### Cell Targeting Agent of TAGE Agent

Examples of cell targeting agents that can be used as the extracellular cell membrane binding moiety of a TAGE agent include, but are not limited to, an antigen binding polypeptide, such as an antibody, a cell penetrating peptide (CPP), a ligand, or any combinations thereof. Further, extracellular cell membrane binding moieties, such as ligands and antigen-binding polypeptides, not only allow for receptor-mediated entry of TAGE agents, but in certain instances, the moieties also mediate the biology of the cell (e.g., by altering intracellular signal transduction pathways), which can be exploited for therapeutic uses. Cell targeting agents are alternatively referred to as extracellular cell membrane binding moiety and are further described in International Publication Nos. WO 2020/198151 and WO 2020/198160, as well as U.S. application Ser. Nos. 17/480,913 and 17/481,056, which are each hereby incorporated by reference.

### EXAMPLES

The invention will be more fully understood by reference to the following examples. They should not, however, be construed as limiting the scope of the invention. All literature and patent citations are incorporated herein by reference.

The below examples describe studies, methods and compositions relating to formulation additives and conditions, and their impact on TAGE stability and editing activity ex vivo.

### Example 1: Method for Detection of Aggregation of Cas:gRNA RNPs by UV/Vis Spectrophotometry and Determination of Optimal Buffer Formulation

Cas ribonucleoproteins (RNPs) are prone to aggregation, especially in standard buffers (e.g., Phosphate Buffered Saline (PBS)) and in physiological conditions (neutral pH and low salt concentrations), which limits their enzymatic activity and alters their cell penetrating properties. Moreover, salt concentration and overall solution tonicity are important factors that affect cells' ability to uptake proteins (D.S. D'Astolfo, et al., Efficient intracellular delivery of native proteins. Cell 161, 674-690 (2015)). Therefore, successful production of TAGE therapeutics may require buffer conditions that maintain or maximize RNPs in a soluble, un-aggregated state during extended storage and maintain or maximize desired uptake properties of the TAGE agent in a cell. In this Example, a method to detect RNP aggregation was developed to identify an optimized formulation buffer having a reduced level of RNP aggregates.

### Turbidity of RNPs in SH Buffer

For this study, RNPs made up of Targeted Active Gene Editing (TAGE) agents (i.e., TAGE agent RNPs) were assessed for aggregation. First, aggregation of RNPs formed from TAGE agents were assessed in SH buffer at 42 degrees Celsius. TAGE agent RNPs were formed by incubating 24 μM of the TAGE agent (Cas9(C80A)-2×NLS or 4×NLS-Cas9(C80A)-2×NLS) with 28.8 μM guide RNA (sgBFP or sgJD98) in SH buffer (20 mM L-Histidine, 100 mM L-Arginine, 200 mM NaCl, 5% w/v sucrose, pH 7.3). The TAGE agent RNPs were incubated at 42 degrees Celsius for 10 minutes, after which the RNP solutions were assessed for turbidity by visual inspection. The results of this study indicated that certain TAGE can aggregate in standard buffers, especially at elevated temperatures and/or at high concentrations. The degree and duration of aggregation was dependent on the identities of the TAGE and the gRNA, but reversible aggregation was observed for each, and irreversible aggregation was observed for the 4×NLS-Cas9(C80A)-2×NLS RNPs,

### RNP Aggregation in PG Buffer Vs SH Buffer as Measured by UV/Vis Absorbance

Next, a method to detect RNP aggregate was developed based on UV/Vis absorbance spectroscopy. Using this method, light scattering, as indicated by a sloping baseline, was used to detect aggregation by TAGE agent RNPs following incubation in PG Buffer (Phosphate Buffered Saline (PBS)+10% glycerol) or SH300 buffer (20 mM L-Histidine pH 7.4, 300 mM NaCl, 100 mM L-Arginine, and 5% w/v sucrose). The aforementioned TAGE agents (Cas9(C80A)-2×NLS or 4×NLS-Cas9(C80A)-2×NLS complexed with one of two gRNAs (sgBFP or sgJD98)) were incubated in PG Buffer or SH300 buffer at 37 degrees Celsius (C) for 10 minutes and then analyzed by UV/Vis absorbance spectroscopy. UV/Vis absorbance spectra from 220-900 nm were measured for each of the RNP samples on a Nanodrop spectrophotometer (ThermoFisher Scientific), with their respective buffers as blanks. The spectra were measured at the standard path length (1 mm), and absorbance measurements were converted to their 1 cm equivalent. The spectra were baseline subtracted using the absorbance at 800 nm.

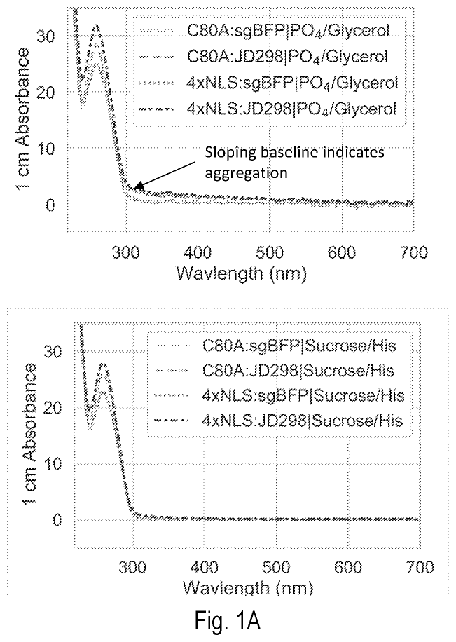

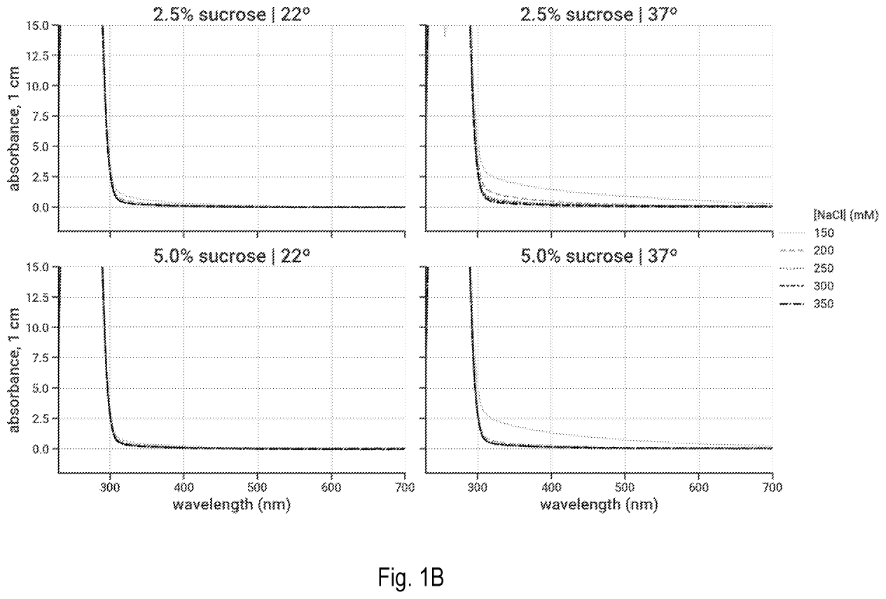

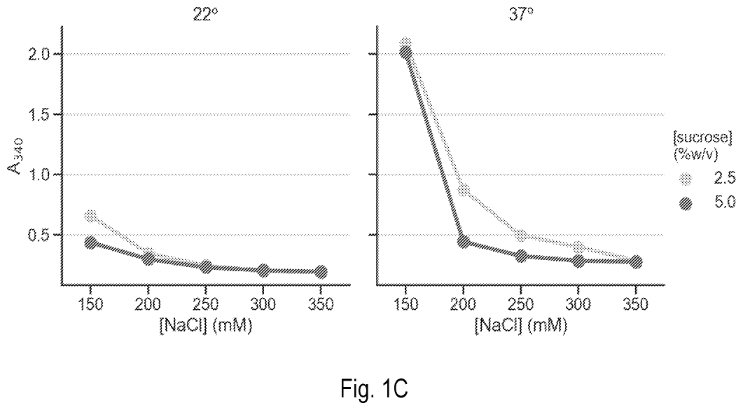

As shown in FIG. 1A, TAGE agents including 4×NLS-Cas9(C80A)-2×NLS displayed a sloping baseline indicative of light scattering was observed with TAGE agent RNPs incubated in PG buffer (FIG. 1A), which indicates that the TAGE agent RNPs aggregated in PG buffer at 37 degrees Celsius. In contrast, a sloping a baseline indicative of aggregation was not observed for TAGE agent RNPs incubated in SH300 buffer (FIG. 1B).

### Impact of Salt and Sucrose on RNP Aggregation as Measured by UV/Vis Absorbance

Next, the impact of salt and sugar concentration on TAGE agent RNP aggregation was assessed. sgBFP single guide RNA was resuspended in a buffer including 5 mM HEPES pH 7.5, 0.1 mM EDTA. This gRNA was re-folded by incubating the gRNA at 70° C. for 5 minutes and then slow cooling the gRNA on the benchtop at room temperature for 10 minutes. Cas9:gRNA complexes (RNPs) were formed by combining this re-folded sgBFP and Cas9(C80A) in a series of various formulations containing 20 mM L-Histidine, 100 mM L-Arginine, sucrose at 2.5 or 5% (w/v), 100-500 mM NaCl, pH 7.4. Each RNP formulation sample was divided into two aliquots. For each RNP formulation, one of the two samples was incubated at room temperature (22° C.), and the other sample was incubated at 37° C. for 10 minutes, and then each was placed on ice. UV/Vis absorbance spectra from 190-900 nm were measured for the RNP samples on a Nanodrop spectrophotometer (ThermoFisher Scientific), with their respective formulations as blanks. The spectra were measured at the standard path length (1 mm), and absorbance measurements were converted to their 1 cm equivalent. The spectra were baseline subtracted using the absorbance at 800 nm.

FIG. 1B shows the absorbance spectra of all samples, shaded according to their salt concentration and separated by their sucrose concentration (2.5% sucrose on the top plot vs 5% sucrose on the bottom plot) and incubation temperature (22° C. on the left plot and 37° C. on the right plot). FIG. 1C shows the absorbance at 340 nm from the spectra in FIG. 1A as a function of a salt concentration. The RNP samples incubated at 37° C. with low NaCl and low sucrose concentrations were observed to have a sloping baseline away from the peak at 260 nm, and a high absorbance at 340 nm, indicative of light scattering by aggregated or precipitated RNP molecules. The samples with high absorbance at 340 nm were visibly cloudy (not shown). The RNP samples with high NaCl and high sucrose concentrations had flat baselines in the near-UV range of the spectra, and thus minimal absorbance at 340 nm.

These results indicate that the TAGE agent RNPs had a reduced level of aggregates in formulations with higher salt and sugar concentrations. For example, a sucrose concentration of 5% and an NaCl concentration of 200 mM displayed minimal absorbance at 340 nm, indicating that sucrose and salt stabilized the TAGE agent RNP from aggregation.

### Example 2: Effect of gRNA Re-Folding and Mg²⁺ on gRNA Elution During SEC

Free guide RNA (i.e., gRNA not in complex with a nucleic acid-guided nuclease) can display aberrant elution on an SEC column (biocompatible silica-based) in standard biochemical buffers. In such instances, the gRNA does not elute as a single peak, which can interfere with the resolution and detection of RNPs in a sample. In this Example, the impact of gRNA re-folding and the presence of Mg²⁺ in the SEC running buffer on the elution of free gRNA were assessed. The gRNAs in this Example were assessed without the presence of a nucleic acid-guided nuclease in the sample.

### Methods

Free gRNA elution was assessed by size exclusion chromatography (SEC)-high performance liquid chromatography (HPLC). The SEC column was an AdvanceBio SEC, 300 Å pore size, 2.7 μm bead diameter, 4.6 mm column diameter, 300 mm column length (Agilent). The mobile phase was 25 mM MES pH 6.0, 500 mM NaCl, with or without 1 mM MgCl₂. The flow rate was 0.25 ml/min. The detector was a diode array detector (Agilent) with a 1 cm path length flow cell. The absorbance at 260 nm (16 nm bandpass) minus 340 nm (16 nm bandpass) was recorded. Each sample was injected via autosampler. The column, buffer, and flow rate were selected to maximize peak resolution.

### Effect of Re-Folding on Guide RNA SEC Elution

First, the impact of re-folding on the elution of free gRNA was assessed by SEC-HPLC. To refold the gRNA, the gRNA was pre-treated under conditions that cause the gRNA to at least partially denature and subsequently renature (i.e., conditions that cause the gRNA to “refold”) prior to loading onto the SEC column.

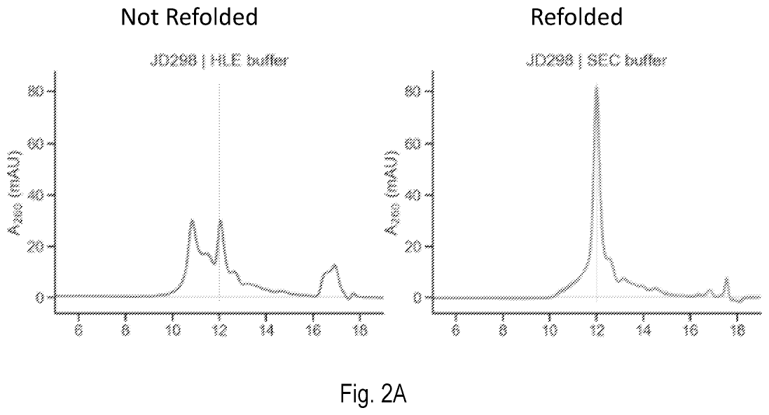

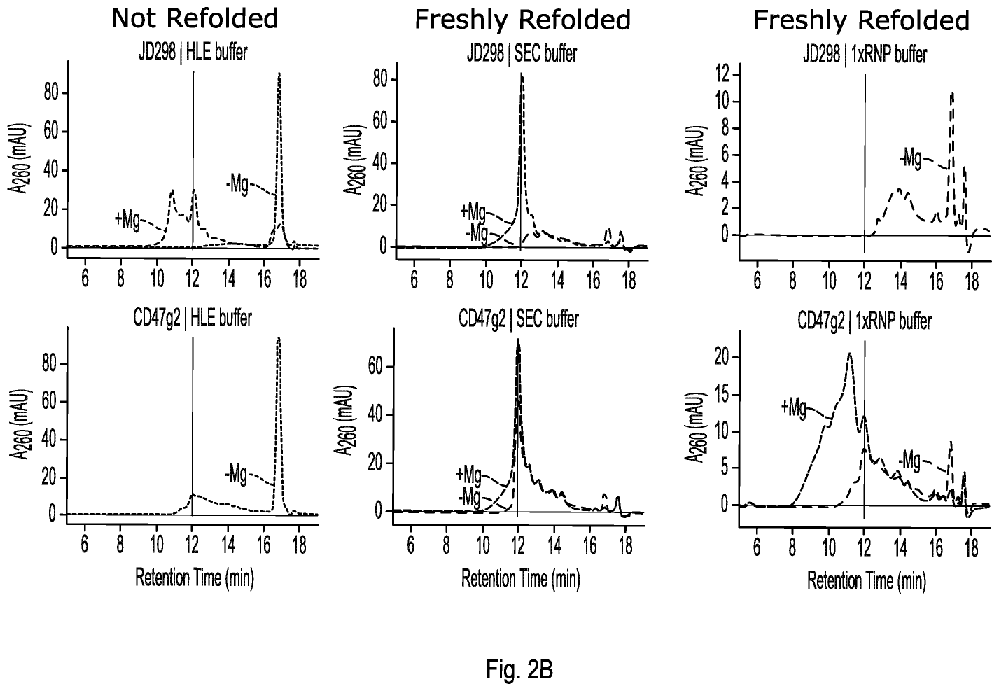

To re-fold the guide RNA, the gRNA was incubated in the indicated buffer for 5 minutes at 70° C. for 5 minutes, then allowed to cool slowly at ambient temperature (22° C.) for 10 minutes. Two different guide RNAs were assessed (JD298). JD298 was incubated in HLE Buffer (5 mM HEPES pH 7.5, 0.1 mM EDTA) or SEC buffer (20 mM HEPES pH 7.5, 200 mM NaCl. 10% v/v glycerol). As shown in FIG. 2A, the majority of the freshly re-folded gRNA eluted as a single peak. In contrast, gRNA that was not subjected to the re-folding process (left panel) had an aberrant elution profile.

### Effect of Mg²⁺ on Free RNA Elution on SEC

Next, the impact of magnesium in the SEC running buffer on the elution of free guide RNA was assessed by SEC-HPLC. gRNAs were assessed with and without re-folding. To re-fold the guide RNA (gRNA), the gRNA was incubated in the indicated buffer at 70° C. for 5 minutes, then allowed to cool slowly at ambient temperature (22° C.) for 10 minutes. Two different guide RNAs were assessed (JD298 and CD47g2). Each gRNA was incubated in HLE Buffer (5 mM HEPES pH 7.5, 0.1 mM EDTA), SEC buffer (20 mM HEPES pH 7.5, 200 mM NaCl. 10% v/v glycerol), or 1×RNP buffer (20 mM HEPES pH 7.5, 200 mM KCl, 5% glycerol, 1 mM MgCl₂). The gRNA was loaded on a SEC column with running buffer (25 mM MES pH 6.0 and 500 mM NaCl) with or without 1 mM MgCl₂. As shown in FIG. 2B, the majority of gRNA eluted as a single peak when the gRNA was re-folded and applied to the SEC column with an SEC running buffer that included MgCl₂.

### Example 3: Method for Detection of Aggregation of Cas:gRNA RNPs by UV/Vis Spectrophotometry and Determination of Optimal Buffer Formulation

When producing RNPs, some samples include free gRNA and/or nucleic acid-guided nucleases (e.g., Cas9) in addition to the properly formed RNP. As demonstrated in Example 2, free gRNA in the sample can have an aberrant elution profile under certain elution conditions, which can interfere with the resolution and detection of RNPs in a sample. Such samples may also have undesirable characteristics such as altered immunogenicity or toxicity due to the properties of free RNA or free Cas protein that differs from those of the RNP. During formation of these complexes, it therefore important to have methods to detect the efficiency of RNP formation, for example by detecting the presence of unbound species alongside the RNP complex.

Based on the buffer and gRNA re-folding conditions identified in Example 2, this Example provides a study that demonstrates a method for detection of free gRNA alongside fully-complexed RNP by size-exclusion chromatography-high performance liquid chromatography (SEC-HPLC) with UV absorbance detection.

Samples were assessed with different molar ratios of Cas9 and sgRNA. RNPs were formed by co-incubation of freshly re-folded sgBFP RNA at 2 μM and Cas9(C80A)-2×NLS at a range of 0-4.8 μM, in a buffer containing 20 mM HEPES pH 7.5, 200 mM KCl, 1 mM MgCl₂, and 5% (v/v) glycerol. These RNP samples were incubated at 37° C. for 10 minutes, then snap-frozen and stored at −80° C. Samples were thawed on ice and kept at 6° C. until injection on the HPLC.

The SEC-HPLC conditions were as follows. The column was an AdvanceBio SEC, 300 A pore size, 2.7 μm bead diameter, 4.6 mm column diameter, 300 mm column length (Agilent). The mobile phase was 25 mM MES pH 6.0, 500 mM NaCl, 1 mM MgCl₂. The flow rate was 0.25 ml/min. The detector was a diode array detector (Agilent) with a 1 cm path length flow cell. The absorbance at 260 nm (16 nm bandpass) minus 340 nm (16 nm bandpass) was recorded. 10 μl of each sample was injected via autosampler. The column, buffer, and flow rate were selected to maximize peak resolution.

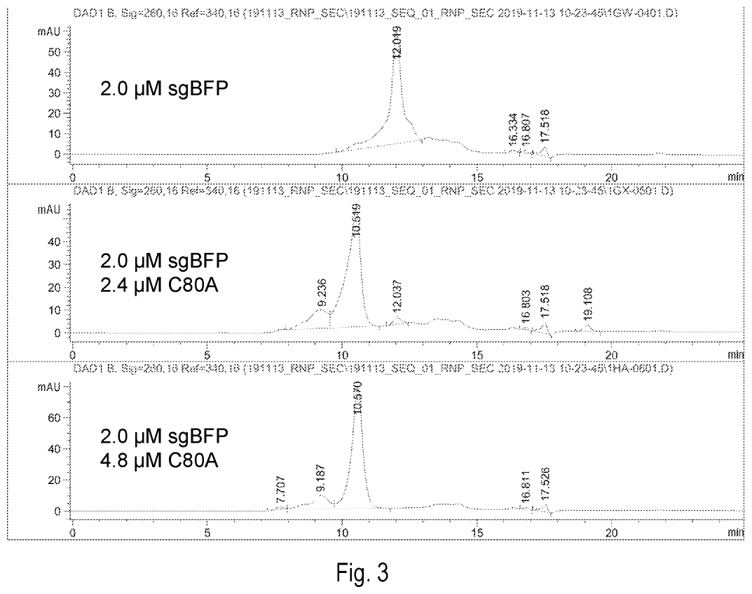

FIG. 3 shows HPLC chromatograms of absorbance at 260 nm for the three RNP samples at different molar ratios of Cas9 and sgRNA or the sgRNA alone, as indicated in the figure. The free RNA (top plot) eluted at ˜12 minutes. The C80A:sgBFP RNP eluted at 10.5-10.6 minutes, with a small shoulder at ˜9.2 minutes. A small free RNA peak was visible with a slight molar excess of Cas9 (middle plot), and no free RNA was visible with 2.4-fold molar excess Cas9 (bottom plot). These results indicate that the NaCl concentration (500 mM) and the inclusion of MgCl₂ maintained well-resolved, symmetric peaks for both RNP and free gRNA.

### Example 4: Anion Exchange does not Effectively Resolve RNP from Free RNA

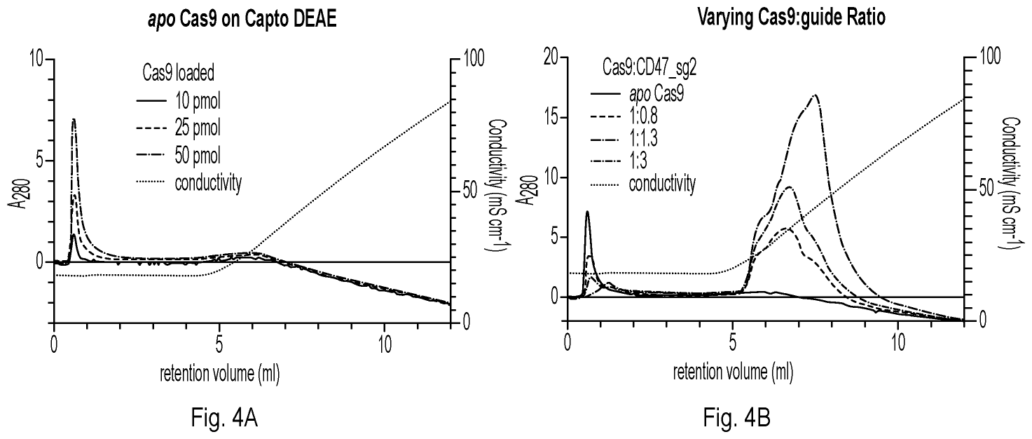

The efficacy of anion exchange chromatography (AIEX) to separate free gRNA from fully-complexed RNPs was assessed in this Example. First, Cas9 without gRNA (i.e., apo Cas9) was applied to an anion exchange column (Capto DEAE) at a level of 10 pmol, 25 pmol, and 50 pmol. The indicated amounts were diluted to 100 μl with Buffer A (25 mM Tris pH 8.5, 100 mM NaCl) and injected onto a 1 ml HiTrap Capto DEAE ImpRes equilibrated in Buffer A. The column was eluted at 1 ml per minute with a 10 ml gradient from 0-100% Buffer B (25 mM Tris pH 8.5, 1.9 M NaCl). As shown in FIG. 4A, Cas9 was not retained and eluted as a single peak at 0.6 ml.

Next, Cas9 was combined with gRNA at a ratio of 1:3, 1:1.3, and 1:0.8 Cas9:gRNA and applied to the anion exchange column. Cas9(C80A):CD47sg2 were prepared at the indicated molar ratios in 1×RNP buffer. The indicated amounts were diluted to 100 μl with Buffer A (25 mM Tris pH 8.5, 100 mM NaCl) and injected onto a 1 ml HiTrap Capto DEAE ImpRes equilibrated in Buffer A. The column was eluted at 1 ml per minute with a 10 ml gradient from 0-100% Buffer B (25 mM Tris pH 8.5, 1.9 M NaCl).

As shown in FIG. 4B, free gRNA could not be resolved from RNPs using anion exchange chromatography.

### Example 5. Cas9 RNP TAGE Stability

To assess how physiological conditions affect Cas9 RNP, the stability of the TAGE agent Cas9(C80A)-2×NLS RNP in mouse blood, plasma, serum, and the tumor microenvironment (TME) was examined. Cas9(C80A)-2×NLS RNP was reconstituted with sgRNA to a final concentration of 5 μM RNP. 1 μl of 5 μM Cas9(C80A)-2×NLS RNP was diluted (final concentration of 1 μM) into mouse blood, plasma, serum, TME fluid, or buffer, isolated just prior to experiment. The given fluid is at a final concentration is 80%. The RNP was incubated in the target tissue for 10, 30, 60, and 120 minutes. Next, each solution was diluted 1:10 into an in vitro DNA cleavage reaction for a final concentration of 100 nM.

To assess DNA cleavage, 100 nM of each Cas9 RNP was incubated for 30 minutes at 37° C. with 100 nM of a dsDNA target. DNA cleavage was normalized to DNA cleavage achieved through incubation in buffer alone (i.e., untreated RNP). 1 μl of 20 mg/ml proteinase K was added to the reaction and incubated for 15 minutes at 50° C. The quenching reaction was held at 4° C. prior to separation on a Fragment Analyzer capillary electrophoresis (CE) instrument. 2 μl of the reaction was diluted with 22 μl of TE buffer and analyzed by capillary electrophoresis, per the manufacturer's recommendations. Cleavage reactions were run in triplicate, and background was subtracted from the band intensities. Percent cleavage was quantified with the following equation: % cleavage=(total mass cleaved product)/(total mass of substrate). The results are expressed as % cleavage relative to untreated RNP.

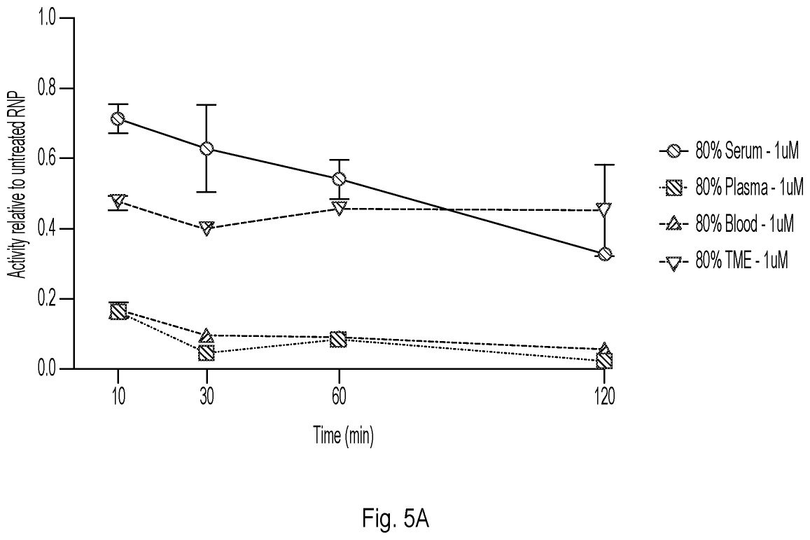

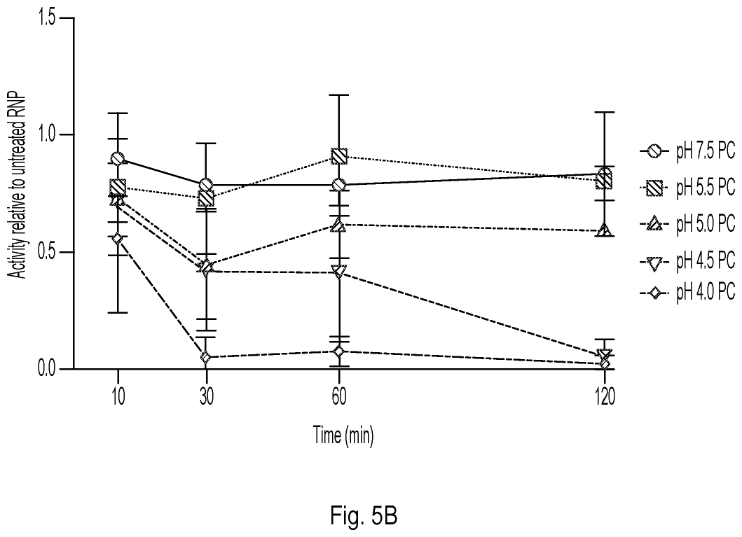

As shown in FIG. 5A, there was a sharp attenuation in DNA cleavage activity of Cas9(C80A)-2×NLS incubated in blood or plasma while a more gradual decline in activity of Cas9(C80A)-2×NLS incubated in serum or the tumor microenvironment

Next, the sensitivity of Cas9(C80A)-2×NLS activity to pH was assessed. Cas9(C80A)-2×NLS RNP was reconstituted with sgRNA to a final concentration of 5 μM RNP. 1 μl of 5 μM RNP was diluted into 50 mM phosphate-citrate buffer with pH adjusted to 4, 4.5, 5, 5.5, or 7.5 to a final concentration of 1 μM of RNP. Acidic pHs are physiologically relevant in the context of, e.g., the tumor microenvironment. The RNP was incubated at the indicated pH for 10, 30, 60, or 120 minutes. The solution was quenched with equal volume 100 mM HEPES pH 7.5 for a final concentration of 100 nM RNP and the RNP was assessed in an in vitro DNA cleavage reaction. The cleavage reaction was performed and data was processed as outlined above. DNA cleavage was normalized to DNA cleavage achieved through a standard reaction (i.e., untreated RNP).

As shown in FIG. 5B, Cas9 RNP in vitro DNA cleavage activity was maintained at a physiological pH. Cas9 RNP TAGE agents retained approximately 50% activity after 1 hour at pH 4.5. This demonstrates that TAGE agent RNPs activity is attenuated by plasma or blood but generally resistant to pH changes.

### Example 6: Effect of gRNAs and Glycerol on the Freeze-Thaw Stability of TAGE Agent RNPs

The impact of different guide RNAs and glycerol on the freeze-thaw stability of TAGE agent RNPs were assessed in this example.

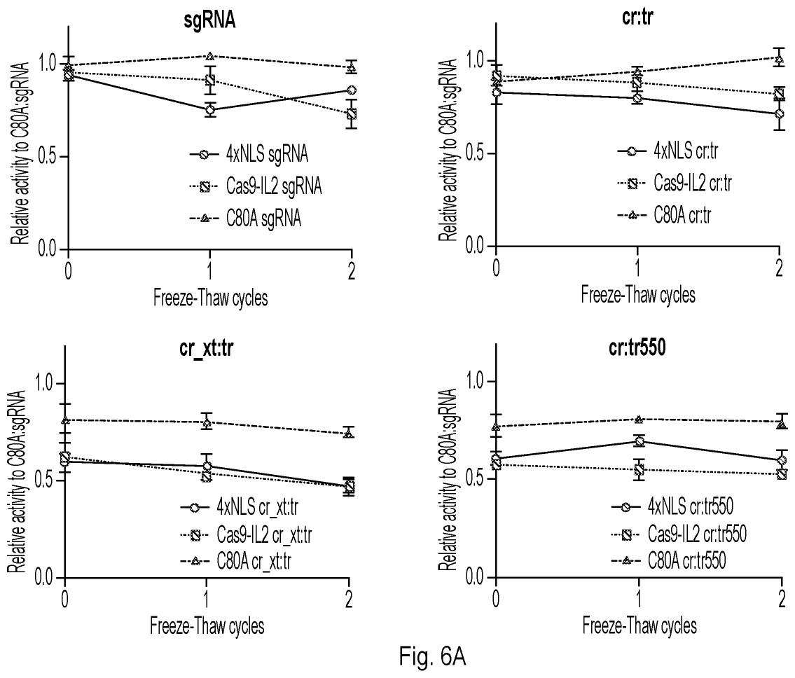

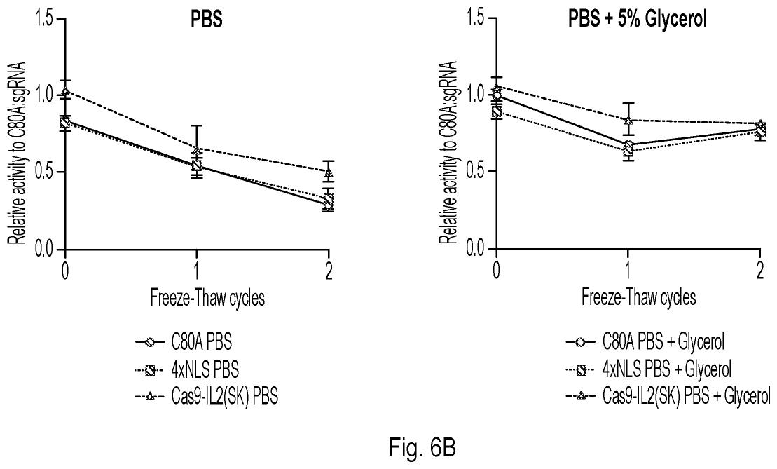

First, the impact of different gRNAs on the freeze-thaw stability of TAGE agent RNPs was assessed. The TAGE agents 4×NLS-Cas9(C80A)-2×NLS (“4×NLS”), IL-2-Cas9-2×NLS, or Cas9(C80A)-2×NLS (“C80A”) were complexed with various commercial guide formulations (a single guide RNA (sgRNA), cr:tr (CRISPR tracer RNA), cr_xt:tr (a guide available through IDT), or cr:tr550 (crispr:ATTO550-tracr)) to form RNPs. The RNPs were then subjected to zero, one, or two freeze-thaw (FT) cycles in storage buffer including glycerol for each freeze-thaw cycles. Following exposure to the FT cycles, the RNPs were tested to assess DNA cleavage activity, as described in Example 5. The results are expressed as cleavage activity relative to Cas9 (C80A):sgRNA activity. As shown in FIG. 6A, guides had a dominant effect on relative cleavage activity, but freeze thaw cycles in glycerol buffer did not severely reduce Cas9 RNP cleavage activity.

Next, the impact of glycerol on the freeze-thaw stability of TAGE agent RNPs was then assessed by measuring the level of RNP cleavage activity following exposure of the RNPs to one or more freeze-thaw cycles. The TAGE agents 4×NLS-Cas9(C80A)-2×NLS (“4×NLS”), Cas9-IL2, or Cas9(C80A)-2×NLS (“C80A”) were complexed with a sgRNA to form RNPs. The RNPs were then subject to zero, one, or two freeze-thaw cycles in either PBS buffer without glycerol or PBS buffer with 5% glycerol. Following exposure to the FT cycles, the RNPs were tested to assess DNA cleavage activity, as described in Example 5. As shown in FIG. 6B, cleavage activity of RNPs stored in PBS buffer without glycerol showed decreasing cleavage activity with each Freeze Thaw cycle. In contrast, RNPs maintained in storage buffer including glycerol showed stabilized Cas9 activity through multiple freeze thaw cycles.

### Example 7: Influence of Re-Folding of gRNA for Cas9 Solubility and Activity

The impact of re-folded gRNA on Cas9 solubility and the cleavage activity of TAGE agent RNPs was assessed in this example.

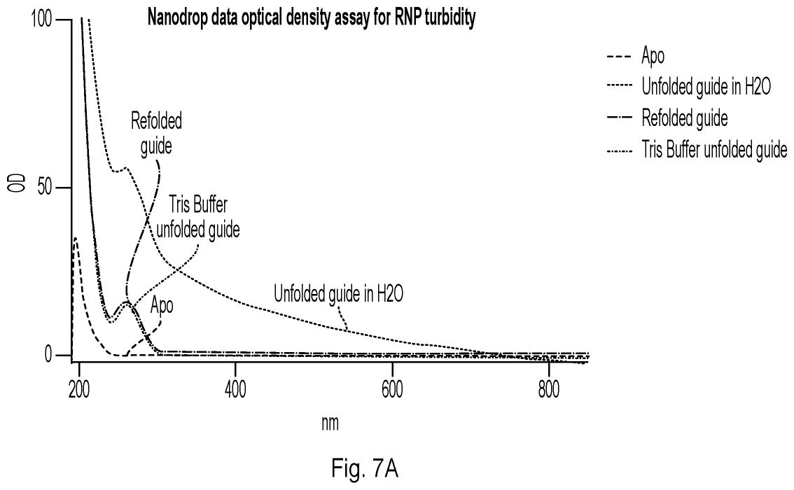

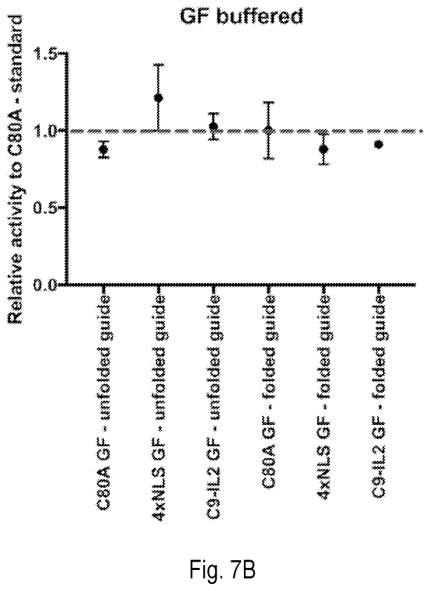

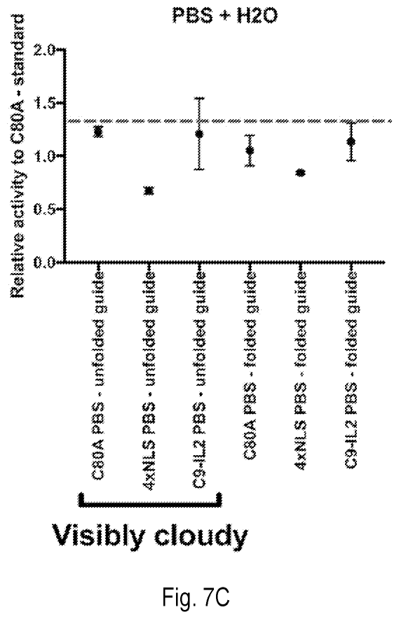

First, the impact of re-folding of sgRNA on Cas9 solubility was assessed using a Nanodrop optical density assay to detect RNP turbidity. To form RNPs, Cas9(C80A)-2×NLS was complexed with re-folded or sgRNA that had not undergone re-folding (“unfolded”), as outlined in Examples 2 and 3. The turbidity was assessed in an optical density assay using a Nanodrop device. In the “re-folded” sgRNA conditions, gRNA was first incubated in buffer at 70° C. for 5 minutes, then allowed to cool slowly at ambient temperature (22° C.) for 10 minutes. The re-folded gRNA was then combined with Cas9 to form an RNP. In the “unfolded” conditions, the sgRNA was not subjected to this process prior to RNP formation. Cas9 without a gRNA (“Apo”) and unfolded gRNA in Tris buffer were also assessed as controls. As shown in FIG. 7A, Cas9 complexed with an unfolded gRNA in H2O generated a larger shoulder from 300 nm onward relative to Cas9 complexed with re-folded gRNA. These results indicated that RNPs complexed with re-folded gRNA had improved solubility relative to RNPs with unfolded gRNA.

Next, the impact of re-folded gRNA on the in vitro DNA cleavage activity of TAGE agent RNPs was assessed. The TAGE agents 4×NLS-Cas9(C80A)-2×NLS (“4×NLS”), Cas9-IL2 (“C9-IL2”), or Cas9(C80A)-2×NLS (“C80A”) were complexed with re-folded or unfolded sgRNA to form RNPs and assessed for DNA cleavage activity in accordance with the protocol in Example 5. RNPs including the TAGE agents and re-folded or unfolded gRNA were reconstituted in standard buffer (SEC buffer (20 mM HEPES pH 7.5, 200 mM NaCl. 10% v/v glycerol); FIG. 7B) or PBS (FIG. 7C) and assessed for activity.

As shown in FIG. 7B, the TAGE agent RNPs retained similar DNA cleavage activity to Cas9 (C80A)-2×NLS in GF buffer with or without gRNA re-folding. In contrast, the TAGE agents, particularly 4×NLS-Cas9-2×NLS, had reduced activity in PBS relative to Cas9(C80A)-2×NLS (FIG. 7C). Cloudy RNP solutions were observed at an RNP concentration of 10 μM when salt concentrations were below PBS (approximately 150 mM), but the cloudiness resolved upon dilution in PBS prior to cleavage.

### Example 8: Impact of Glycerol on TAGE Editing in Primary Cells

In Examples 8 to 13, formulation additives were assessed for their impact on the genome editing activity of Targeted Active Gene Editing (TAGE) agents ex vivo.

In this Example, the impact of glycerol on ex vivo editing by TAGE agents was assessed in the context of primary cell editing. Ribonucleoproteins were prepared from TAGE agents including Cas12a (WT), Cas12a(WT)-4×NLS, En-Cas12a, and Cas12a ultra. Each RNP Was co-incubated with primary murine fibroblast cells in one of two glycerol-containing buffers: (1) a buffer including 1.25% glycerol with cells or (2) a buffer including 6.25% glycerol with cells. The percentage of fibroblast cells that were edited by the TAGE was measured using T7 Endonuclease I assay.

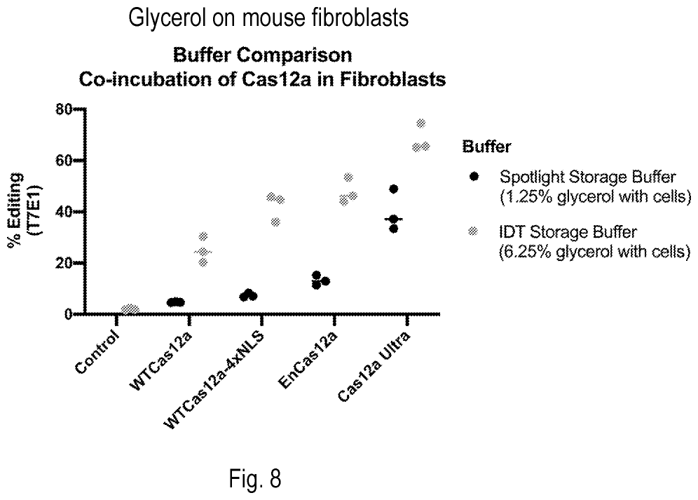

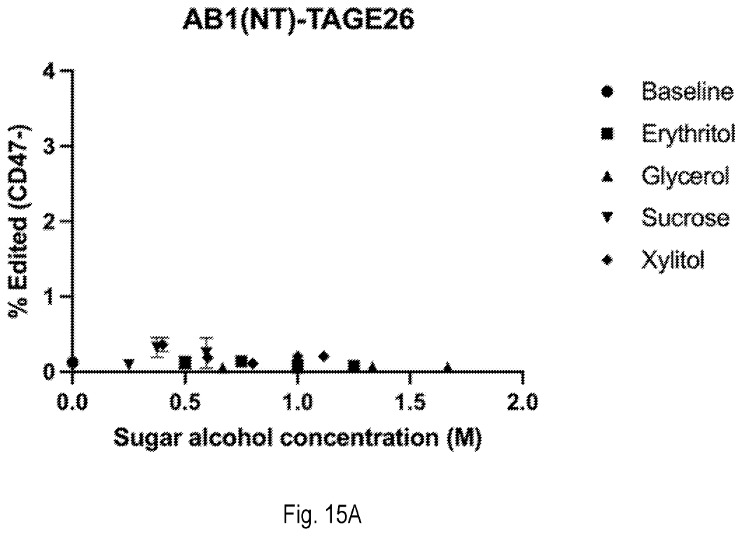

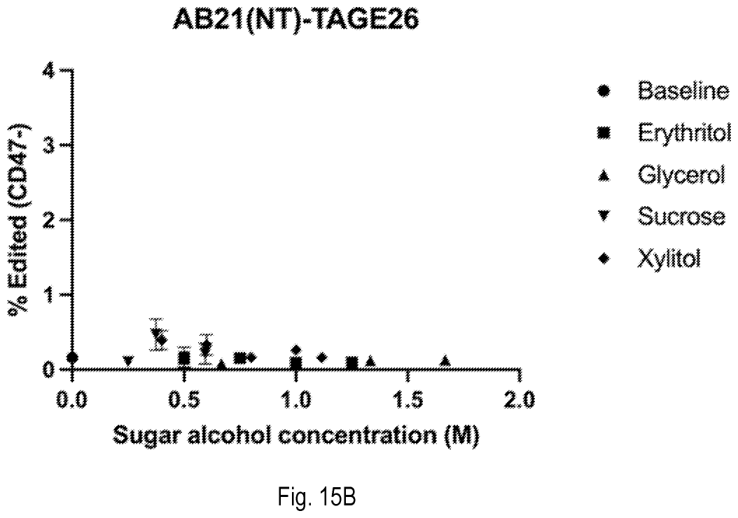

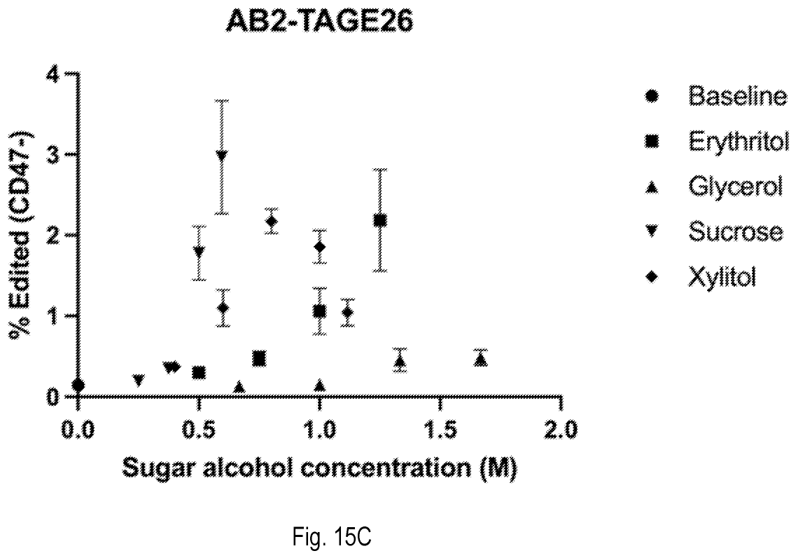

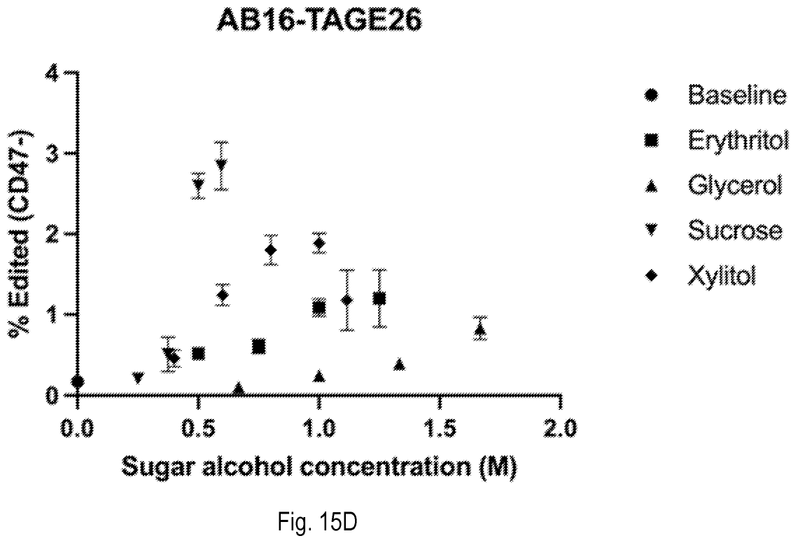

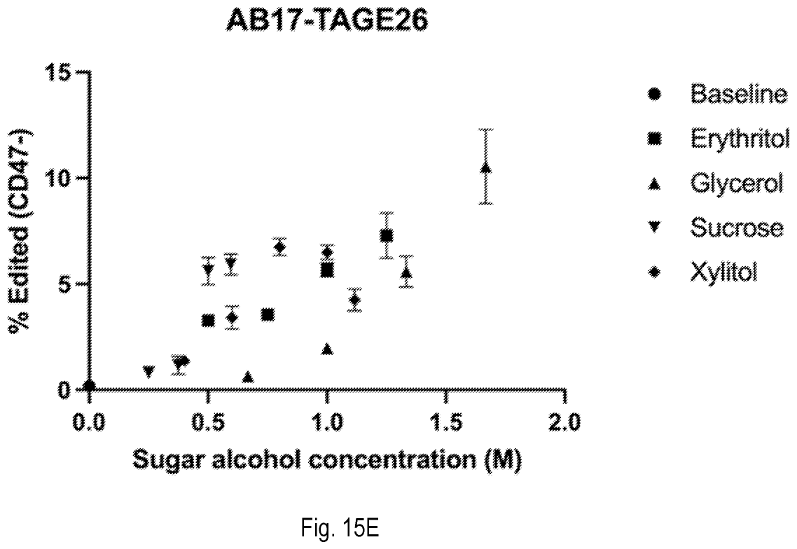

As shown in FIG. 8, TAGEs co-incubated with cells in a buffer including 6.25% glycerol (IDT storage buffer in FIG. 8) resulted in a higher percentage of edited fibroblasts relative to the percentage of edited fibroblasts arising from co-incubation with TAGE in buffer including 1.25% glycerol. In further experiments using human T cells, peak editing ex vivo was observed with 7-10% glycerol (e.g., see Example 10), or 15-16% glycerol (e.g., see Example 10, or FIG. 15E), depending on the TAGE and the salt concentration.

### Example 9: Impact of Chloroquine on TAGE Editing in Primary Cells

In this example, the impact of chloroquine on ex vivo editing by TAGE agents was assessed. RNP TAGE agents including Cas9-2×NLS:sgBFP, 4×NLS-Cas9-2×NLS, Cas9-2×NLS-SpyCatcher-4×NLS, or IL-2-SpyTag:Cas9-2×NLS-SpyCatcher-4×NLS. A guide RNA targeting CD47 (or a non-targeting control, sgBFP) was associated with the respective TAGE agents to form the ribonucleoproteins. The RNPs were co-incubated with PBMCs for 1 hour followed by incubation with 0 μM chloroquine, 10 μM chloroquine, 30 μM chloroquine, or 100 μM chloroquine for 24 hours. The percentage of T cells that were edited by each TAGE was measured using a phenotypic readout detecting the loss of surface CD47 by flow cytometry.

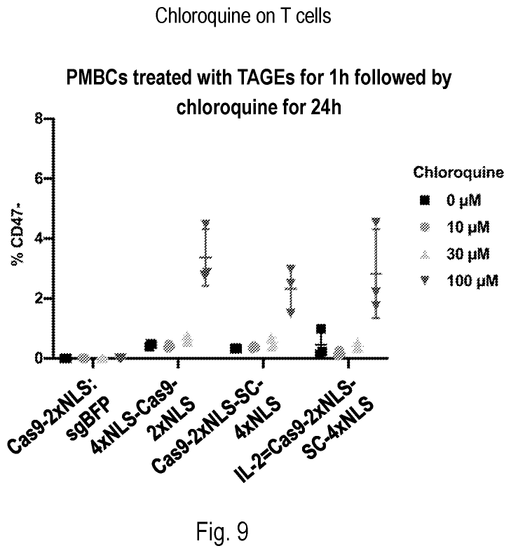

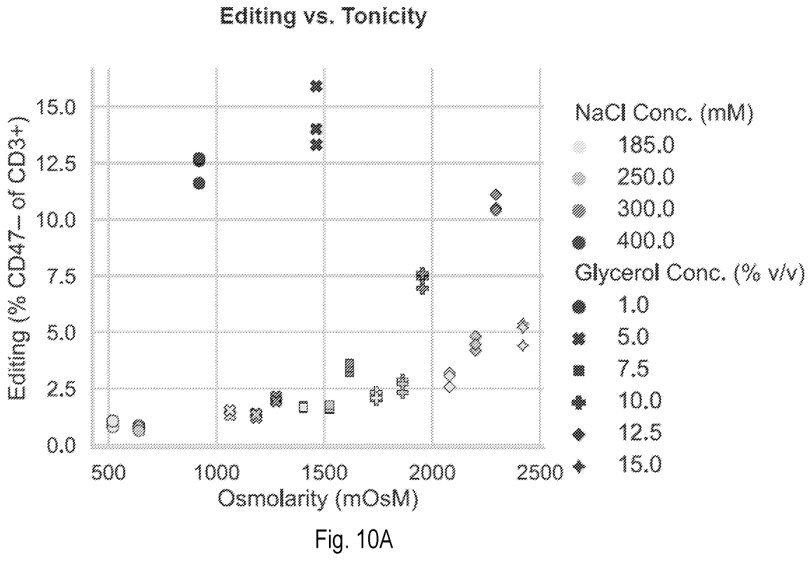

As shown in FIG. 9, addition of chloroquine to the co-incubation reaction increased editing of T cells by TAGE RNPs.

### Example 10: Impact of Glycerol and Salt on TAGE Editing in Primary Cells

In this example, the impact of glycerol and salt on ex vivo editing by TAGE agents was assessed. Ribonucleoproteins were prepared from TAGE agents including 4×NLS-Cas9-2×NLS. A guide RNA targeting CD47 was associated with the respective TAGE agents to form ribonucleoproteins. 3.75 μM RNP was co-incubated with primary human T cells for 1 hour in a range of buffers including salt (e.g., NaCl) and glycerol. Buffers having a range of salt concentrations (185 mM NaCl, 250 mM NaCl, 300 mM NaCl, or 400 mM NaCl) and glycerol concentrations (1%, 5%, 7.5%, 10%, 12.5%, or 15% w/v glycerol) were assessed, resulting in co-incubation conditions with a range of osmolarities. The cells were washed after one hour to remove the additives and RNPs. The percentage of T cells that were edited under each condition was measured using a phenotypic readout detecting the loss of surface CD47 by flow cytometry. Additionally, the levels of live cells per mL 24 hours after co-incubation in the indicated buffer was assessed for each buffer condition.

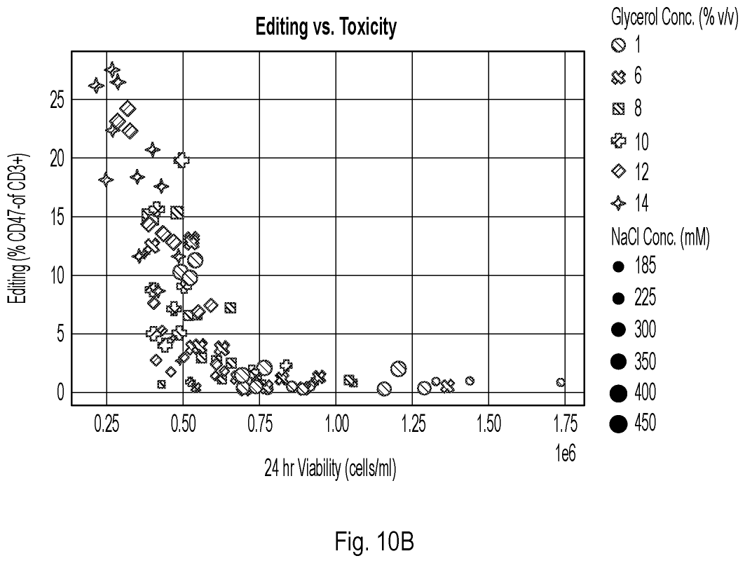

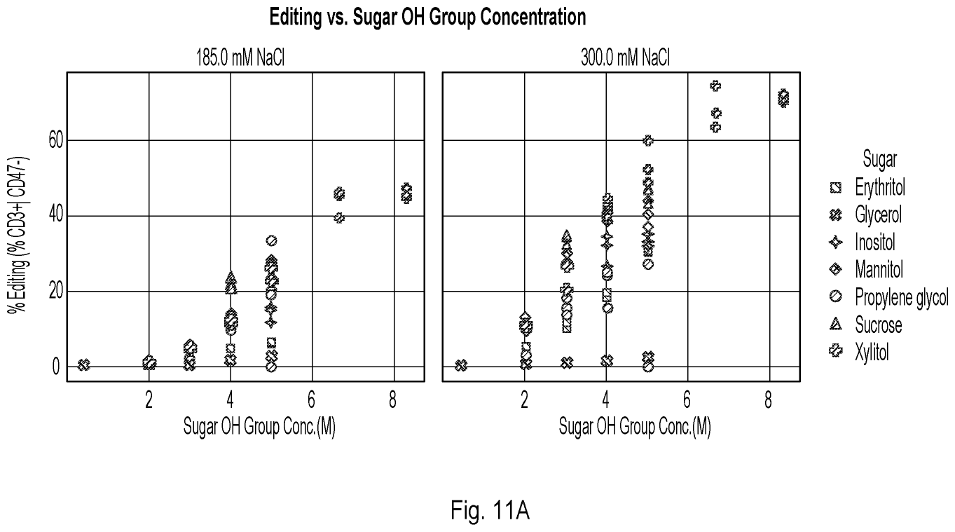

Genome editing was assessed as a function of osmolarity, salt concentration and glycerol concentration. As shown in FIG. 10A, higher percentages of edited cells were observed in solutions having a higher osmolarity. Increasing the level of salt and/or glycerol in the co-incubation buffer resulted in increased genome editing in cells. Further these results indicated that glycerol and sodium chloride had a synergistic effect on genome editing (e.g., FIG. 10A, data point at 300 mM NaCl with 12.5% glycerol had a synergistic effect on editing as compared to 300 mM NaCl with 5% glycerol, or 12.5% glycerol with 185 mM NaCl). Glycerol and salt conditions that displayed the highest increase in editing also reduced the number of live cells per mL over 24 hours, indicative of conditions having higher toxicity in cells (FIG. 10B).

### Example 11: Impact of Salt and Sugar Alcohols/Sugar on TAGE Editing in Primary Cells

In this example, the impact of salt and various polyols (e.g., sugar alcohols) or sugar on ex vivo editing by TAGE agents was assessed. Ribonucleoproteins were prepared from TAGE agents including 4×NLS-Cas9-2×NLS. A guide RNA targeting CD47 was associated with the respective TAGE agents to form ribonucleoproteins. 3.75 μM RNP was co-incubated with primary human T cells for 1 hour in buffers including salt (e.g., NaCl) and a sugar (e.g., sucrose) or a polyol (e.g., propylene glycol, glycerol, erythritol, xylitol, mannitol, inositol). Buffers having different salt concentrations (185 mM NaCl or 300 mM NaCl) and sugar alcohol or sugar concentrations (0.4 mM, 0.8 M, 1.2 M, 1.6 M, 2.0 M, or 2.4 M) were assessed. The cells were washed after one hour to remove additives and RNPs. The percentage of cells that were edited under each condition was measured using a phenotypic readout detecting the loss of surface CD47 by flow cytometry. Additionally, the levels of live cells per mL 24 hours after co-incubation in the indicated buffer was assessed for each buffer condition.

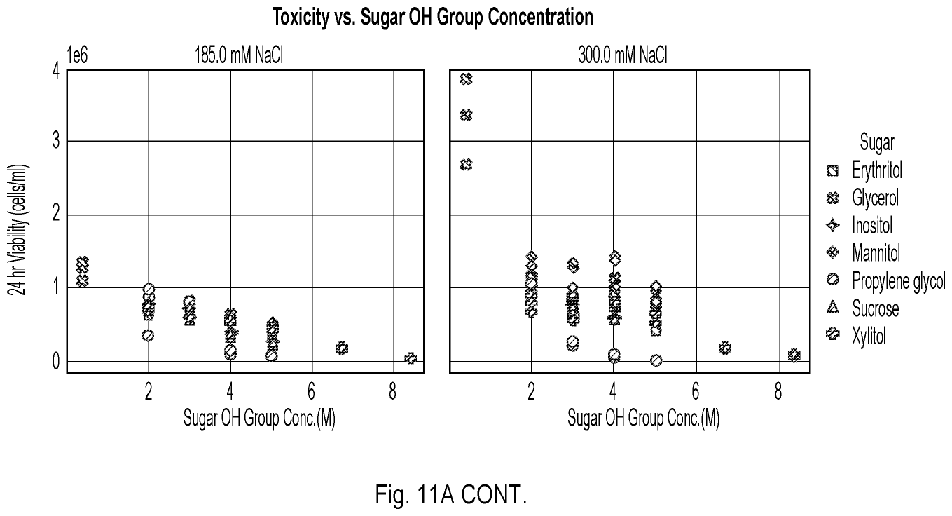

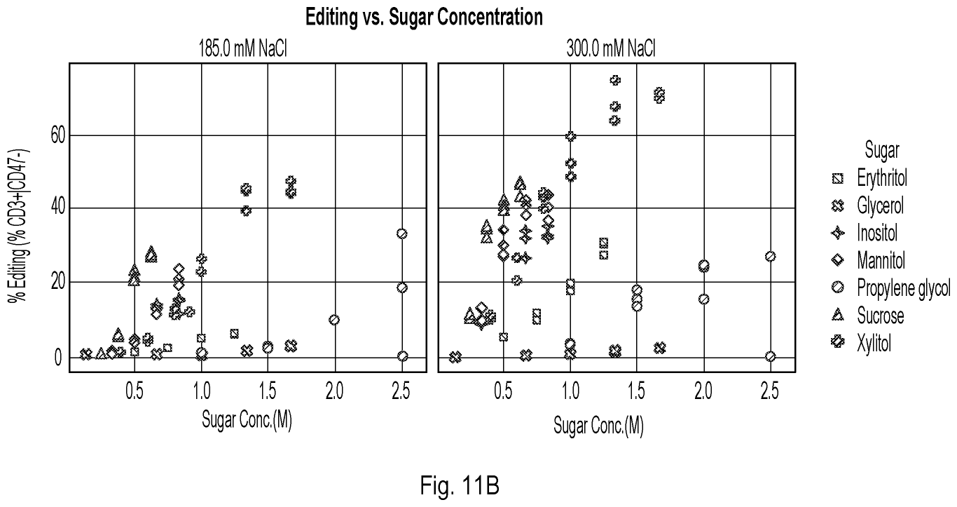

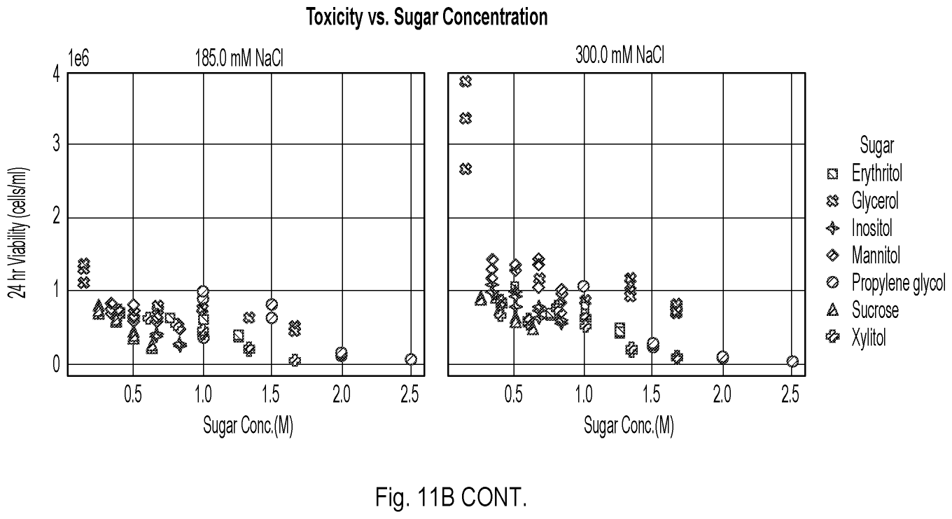

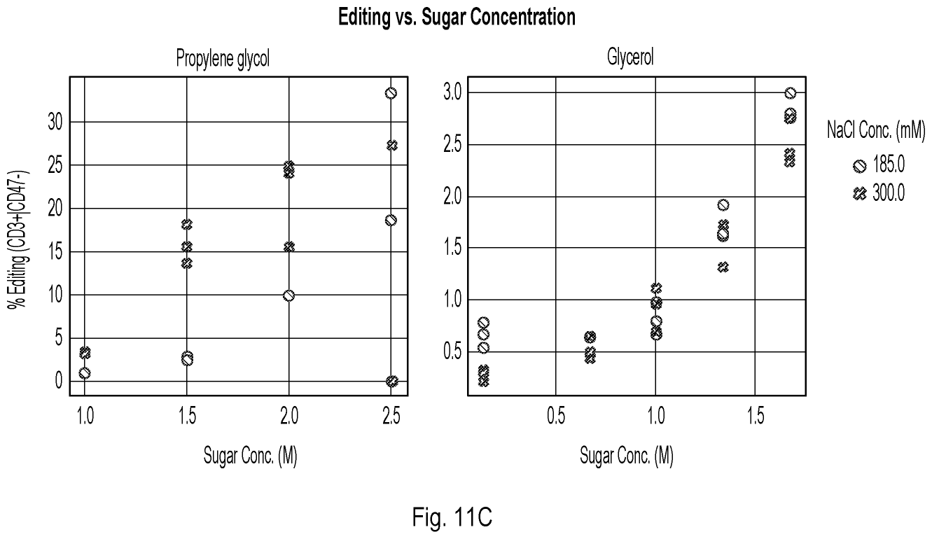

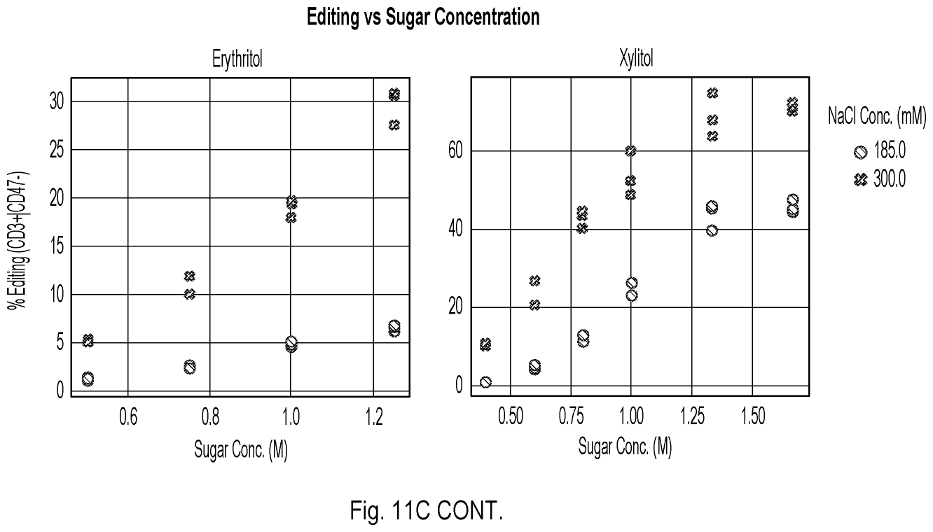

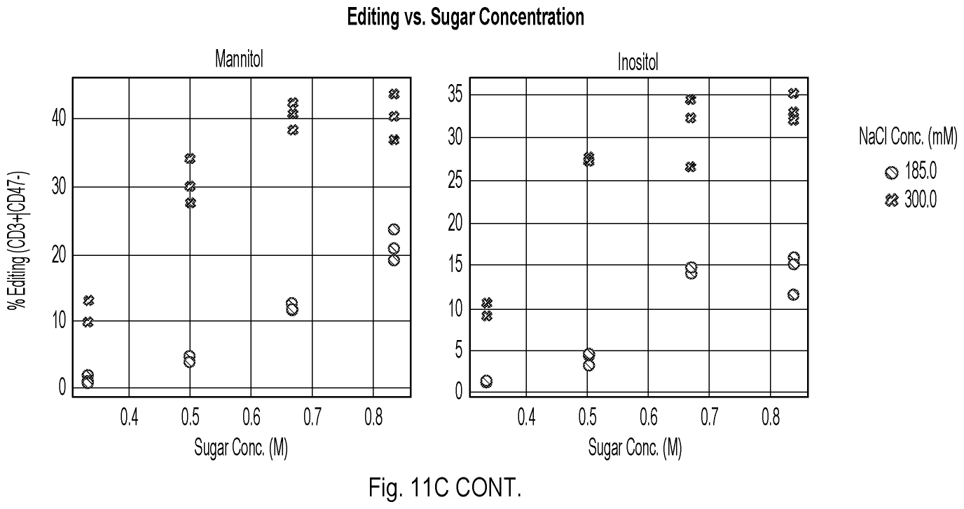

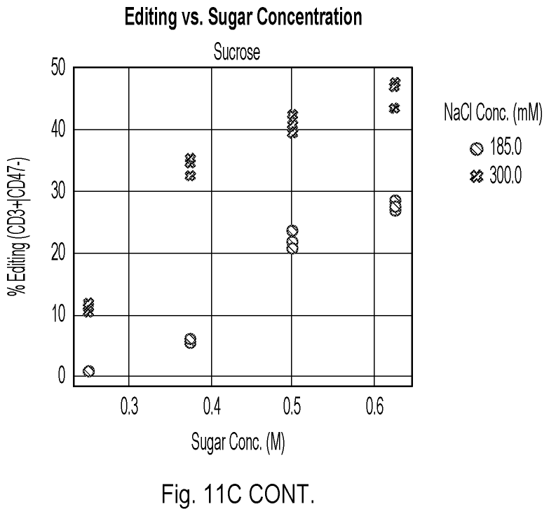

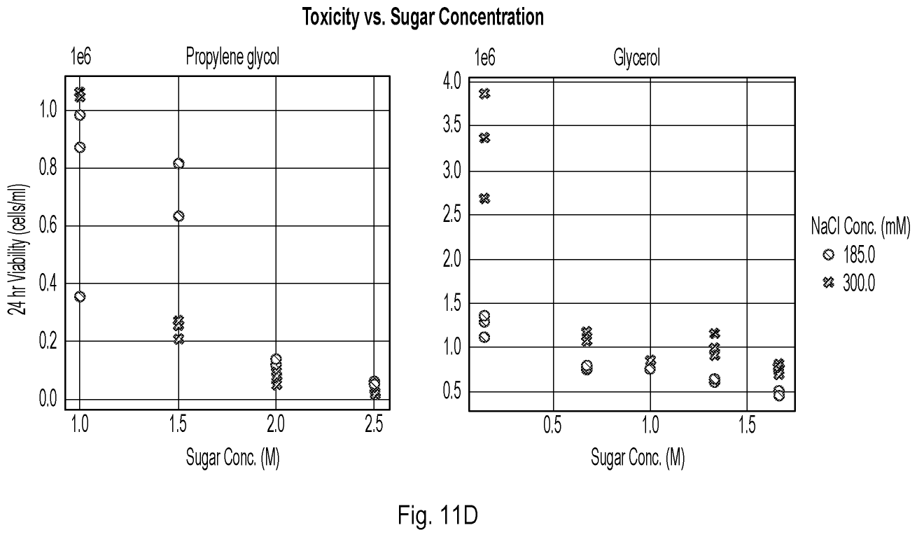

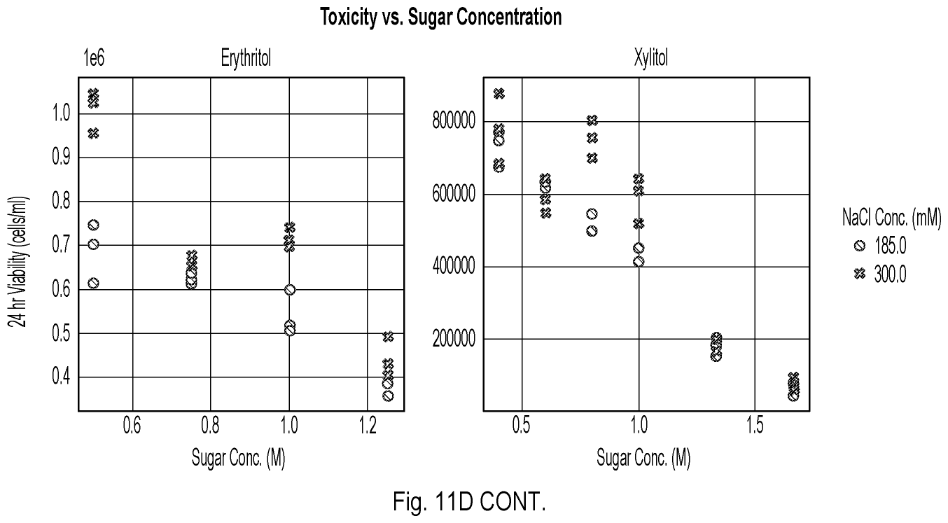

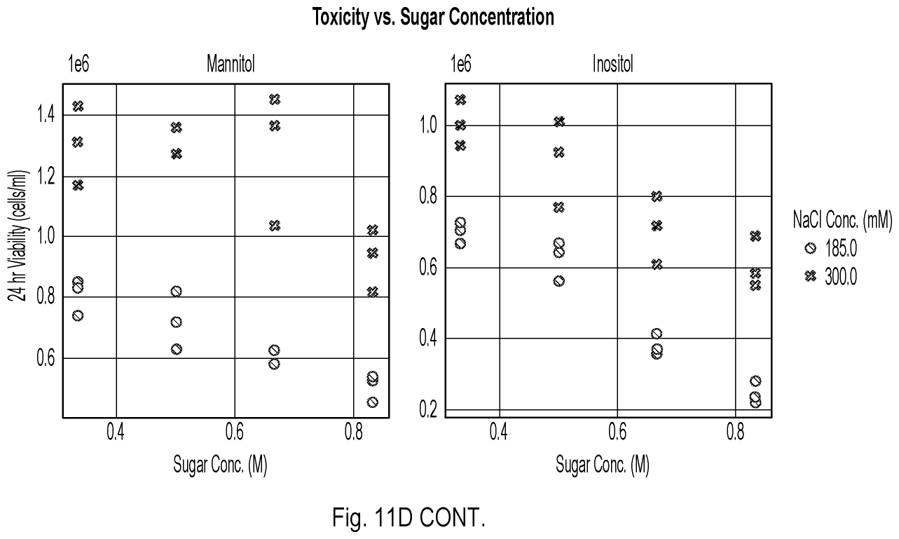

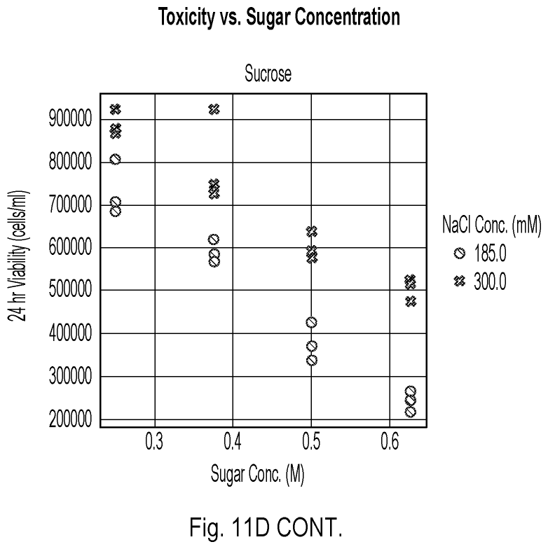

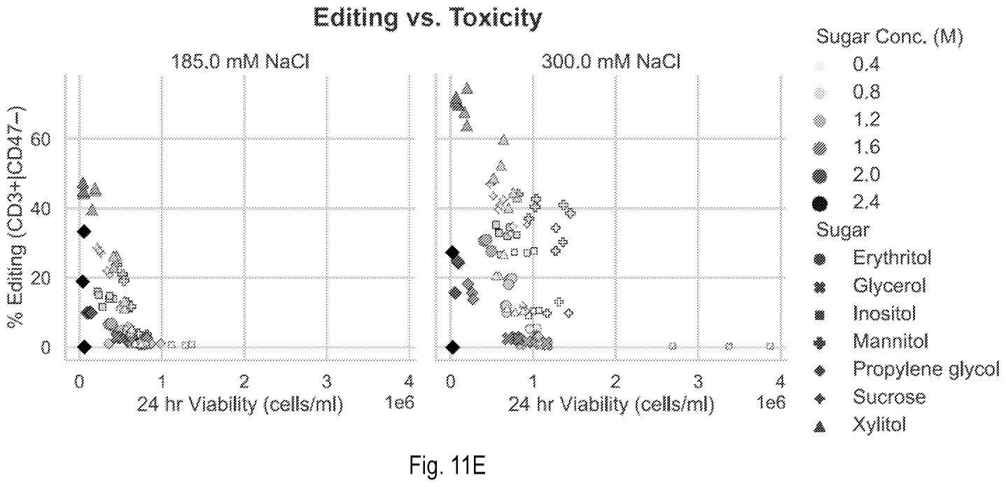

Genome editing was assessed as a function of salt concentration and sugar/polyol OH group concentration (FIG. 11A) or overall sugar molar concentration (FIGS. 11B and 11C). As shown in FIGS. 11A-11C, the percentage of edited cells increased with increasing levels of salt and sugar/polyol concentrations, with over 50% of cell editing observed in some conditions (e.g., 300 mM NaCl and ≥1 M xylitol; FIG. 11B and FIG. 11C). The level of editing varied with the sugar, indicating that both the concentration of the sugar and identity of the sugar or polyol were factors that influenced editing levels. In some instances, salt and sugar/polyol conditions that increased editing also reduced the number of live cells per mL 24 hours after co-incubation, indicative of conditions having higher toxicity (FIGS. 11D and 11E). As shown in FIG. 11E, toxicity was not equivalent across all sugars or polyols.

These results indicate that certain sugars or polyols (e.g., sugar alcohols), such as xylitol, glycerol, or sucrose, in combination with NaCl, can increase TAGE editing efficacy.

### Example 12: Impact of Protamine on TAGE Editing in Primary Cells

In this example, the impact of a cationic polymer (e.g., protamine) with or without glycerol on ex vivo editing by RNP TAGE agents was assessed. Ribonucleoproteins were prepared from TAGE agents including 4×NLS-Cas9-2×NLS or AsCas12a. A guide RNA targeting CD47 was associated with the respective TAGE agents to form ribonucleoproteins. 3.75 μM RNP was co-incubated with primary human T cells for 1 hour in buffers including protamine with and without glycerol. Buffers having different glycerol concentrations (1%, 5%, or 10%) and protamine concentrations (between 0-1.25 M) were assessed. The cells were washed after one hour to remove additives and RNPs. The percentage of T cells that were edited under each condition was measured using a phenotypic readout detecting the loss of surface CD47 by flow cytometry. Additionally, the levels of light scattering at 340 nm was assessed to detect aggregate formation.

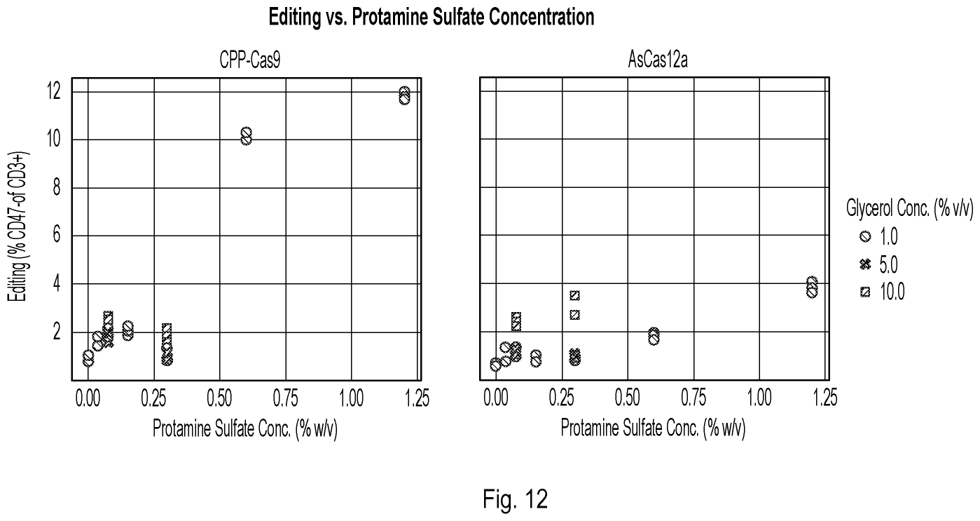

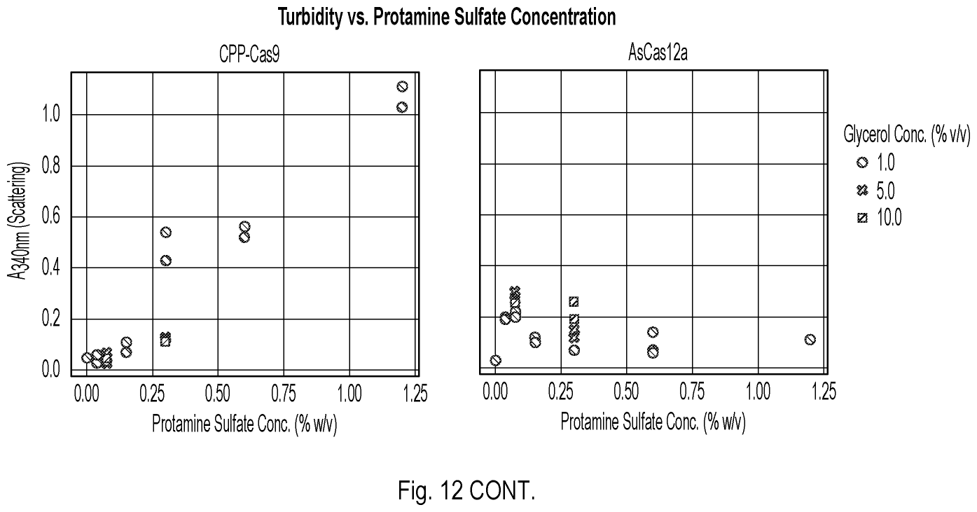

Genome editing was assessed as a function of glycerol and protamine concentration (FIG. 12; left panel). As shown in FIG. 12, protamine caused aggregation and increased editing by 4×NLS-Cas9-2×NLS at low glycerol concentrations. In contrast, protamine did not cause appreciable Cas12a aggregation and showed limited benefits for Cas12a editing.

### Example 13: Impact of Poly-Glutamic Acid on TAGE Editing in Primary Cells

In this example, the impact of an anionic polymer (e.g., poly-glutamic acid) on ex vivo editing by TAGE agents was assessed. Ribonucleoproteins were prepared from TAGE agents including Cas9 or AsCas12a. A guide RNA targeting CD47 was associated with the respective TAGE agents to form ribonucleoproteins. 3.75 μM RNP was co-incubated with primary PBMCs for 1 hour in buffers including poly-glutamic acid (PGA) of different sizes (1500-5500 Da or 15,000 Da). The cells were washed after one hour to remove additives and RNPs. The percentage of T cells that were edited under each condition was measured using a phenotypic readout detecting the loss of surface CD47 by flow cytometry.

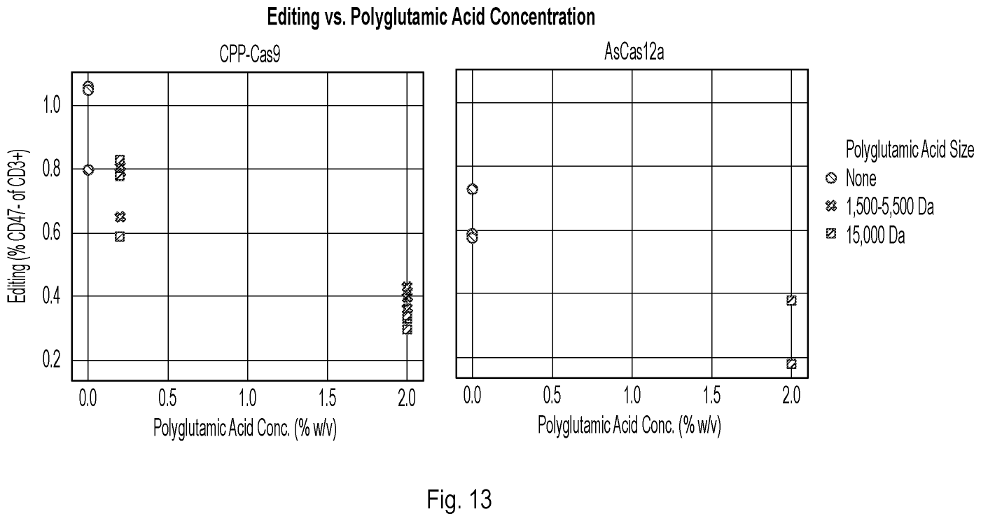

Genome editing was assessed as a function of PGA concentration (FIG. 13). As shown in FIG. 13, PGA inhibited editing by co-incubation, including with lower concentrations and different PGA types.

### Example 14: T Cell Gene Editing by TAGE in Presence of Sucrose

In this example, the impact of the sucrose on editing of primary human T cells by TAGE containing an antibody was assessed.

Antibodies were conjugated to Cas9 using the SpyTag/SpyCatcher system, and a guide RNA targeting CD47 was associated with the respective TAGE agents to form ribonucleoproteins (RNPs). RNPs at the indicated concentration were co-incubated with primary human T cells for 1 hour in buffer including 0.5 M sucrose. The cells were washed after one hour to remove additives and RNPs. The editing percentage under each condition was measured using a phenotypic readout detecting the loss of surface CD47 by flow cytometry. TAGE26 is Cas9-2×NLS-SpyCatcher-4×NLS, and TAGE26 was unconjugated to an antibody.

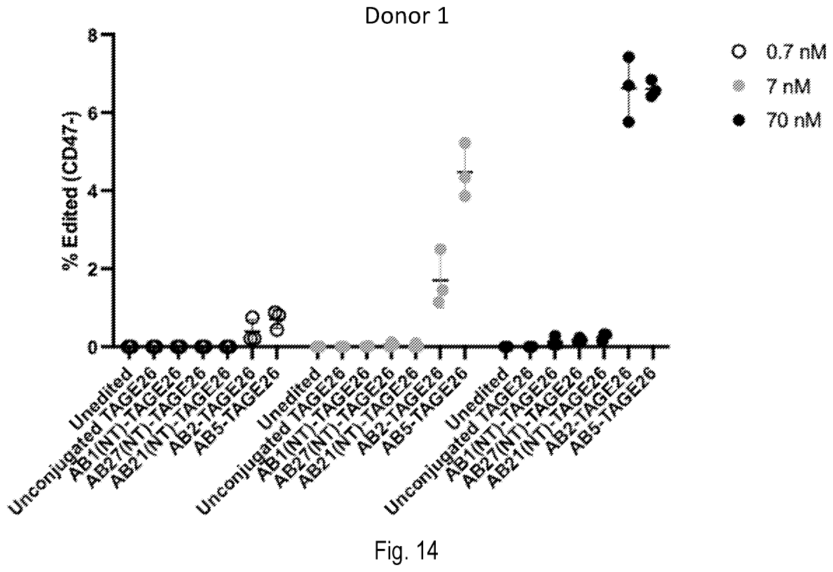

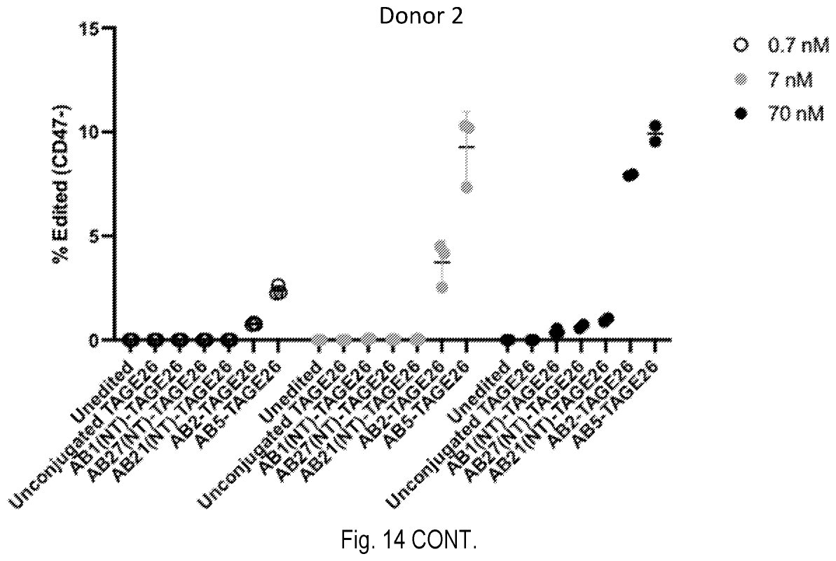

Data is shown from 2 separate human donors (“Donor 1” and “Donor 2” in FIG. 14). Each data point represents a biological replicate. For each group, the horizontal line represents the mean and the error bars represent the standard deviation of the mean. FIG. 14 provides results showing that sucrose improves editing as a formulation additive (sucrose concentration was 0.5 M).

Non-targeting RNPs (i.e., TAGE with non-T cell specific antibodies) were generally unable to edit (similar to the TAGE26 without an antibody), whereas the T cell specific RNPs (AB2-TAGE26 and AB5-TAGE26) were able to edit, with increased editing observed with increased concentration of TAGE. Further, a formulation additive (0.5 M sucrose) can enable editing with TAGE concentrations as low at 0.7 nM.

### Example 15: T Cell Specific Editing by TAGE in Presence of Various Sugar Alcohols and Sugar

In this example, the impact of different sugar alcohols on editing of primary human T cells by TAGE containing T cell specific antibodies was assessed.

Antibodies were conjugated to Cas9 using the SpyTag/SpyCatcher system, and complexed with a guide RNA targeting CD47, thus forming ribonucleoproteins (RNPs). RNPs at the indicated concentration were co-incubated with primary human T cells for 1 hour in T cell media with no additive (Baseline) or with erythritol, glycerol, sucrose, or xylitol at various concentrations ranging from 0.25 to 1.7 M, as described in FIG. 15. Five different TAGE agent were tested. TAGE agents (RNPs) that do not bind to T cells (TAGE agents with non-T cell targeting antibodies) included AB1(NT)-TAGE26 and AB21(NT)-TAGE26 (FIGS. 15A and 15B, respectively). T cell specific TAGE agents (RNPs) tested included AB2-TAGE26, AB16-TAGE26, and AB17-TAGE26 (FIG. 15C-15E). The cells were washed after one hour to remove additives and RNPs. The percentage of cells that were edited under each condition was measured using a phenotypic readout detecting the loss of surface CD47 by flow cytometry. Each data point represents the mean of biological triplicates, and the error bars represent the standard deviation of the mean.

As described in the data in FIGS. 15A-15E, generally each of the tested sugars increased editing by an RNP containing a T cell specific TAGE, in comparison to non-targeting TAGE. FIGS. 15A-15E also show the concentration of additives that yielded the highest editing varied for different sugar alcohols. Some additives yielded higher editing than others at a given concentration.

### Example 16: Impact of Glycerol or Sucrose on Cell Editing by TAGE

In this example, the impact of the presence of sucrose or glycerol as an additive on cell specific editing of primary human T cells by TAGE containing an antibody was assessed.

Antibodies were conjugated to Cas9 using the SpyTag/SpyCatcher system and complexed with a guide RNA targeting CD47 was associated with the respective TAGE agents to form RNPs. Non-specific binding TAGE agent RNPs included AB1(NT)-TAGE26 and AB21(NT)-TAGE26, while T cell specific TAGE agent RNPs tested included AB2-TAGE26, AB16-TAGE26, and AB17-TAGE26.

RNP TAGE agents were co-incubated with primary human T cells for 1 hour in T cell media with no additive (Baseline), 0.5 M sucrose, or 1.67 M glycerol. The cells were washed after one hour to remove additives and RNPs. The percentage of cells that were edited under each condition was measured using a phenotypic readout detecting the loss of surface CD47 by flow cytometry. Each data point represents a biological replicate. For each group, the horizontal line represents the mean and the error bars represent the standard deviation of the mean.

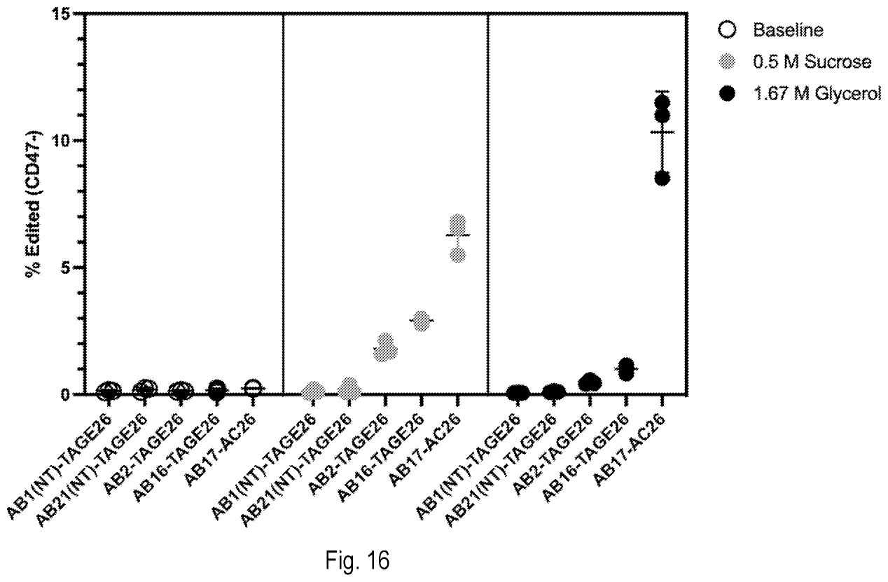

The results in FIG. 16 show that the presence of 1.67 M glycerol resulted in a smaller increase in editing with AB2-TAGE26 and AB16-TAGE26, but a more significant increase in editing with AB17-AC26. The presence of 0.5 M sucrose increased cell specific editing for all cell specific (i.e., T cell binding antibodies) TAGE tested, as shown in FIG. 16.

Thus, some combinations of formulation and antibody had more potent effects on editing than other combinations. For example, 1.67 M Glycerol caused a mild increase in editing with AB2-TAGE26 and AB16-TAGE26, but a much larger increase in editing with AB17-AC26. Editing with AB17-AC26 in 1.67 M glycerol was higher than AB17-AC26 in 0.5 M sucrose, while editing with AB2-AC26 and AB16 AC26 was higher with 0.5 M sucrose than 1.67 M glycerol.

### Example 17: Impact of Sucrose and Salt on Cell Editing by TAGE

In this example, the impact of sucrose and salt (NaCl) on editing of primary human T cells by TAGE containing T cell specific antibodies was assessed.

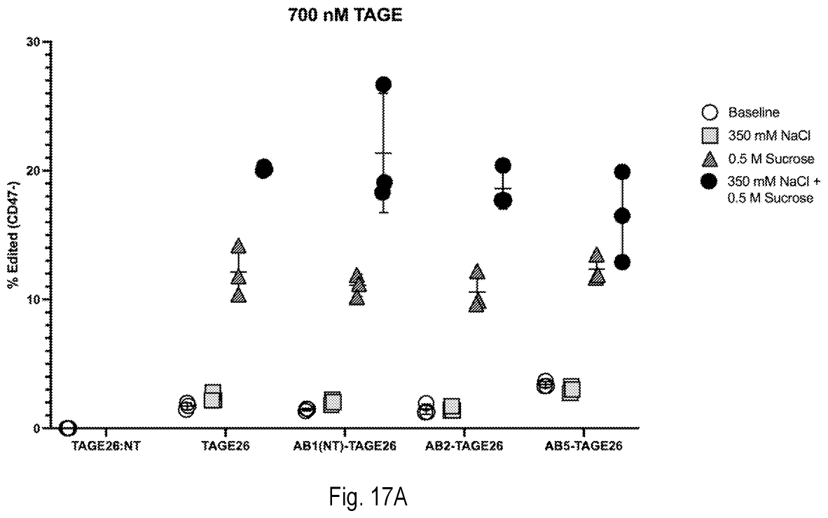

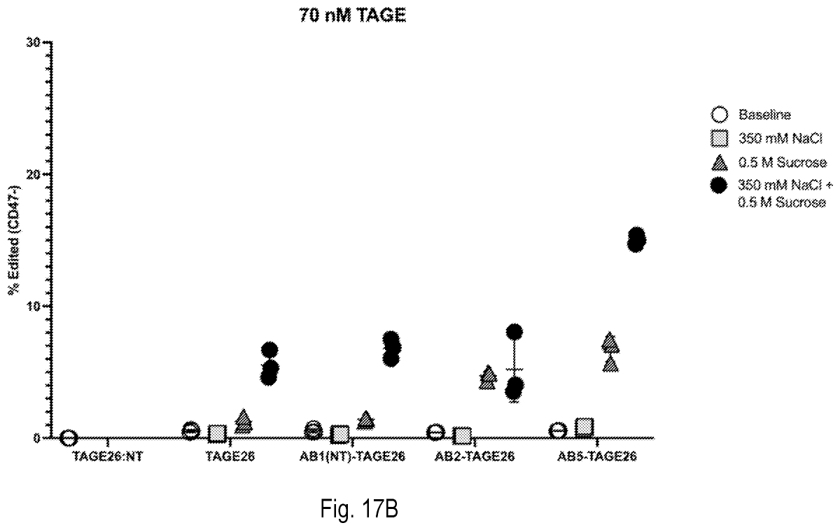

Antibodies were conjugated to Cas9 using the SpyTag/SpyCatcher system and complexed with a guide RNA targeting CD47 was associated with the respective TAGE agents to form an RNP. RNPs were co-incubated with primary human T cells for 1 hour in T cell media with no additive (Baseline), 350 mM NaCl, 0.5 M sucrose, or 350 mM NaCl and 0.5 M sucrose. RNPs tested included a non-targeting TAGE (TAGE26:NT) and a TAGE without an antibody (TAGE26). RNPs containing T cell specific antibodies are described in FIG. 17 as AB2-TAGE26 and AB5-TAGE26. The non-T cell specific antibody-containing TAGE agent used as a negative control is described as AB19(NT)-TAGE26.

The cells were washed after one hour to remove additives and RNPs. The percentage of cells that were edited under each condition was measured using a phenotypic readout detecting the loss of surface CD47 by flow cytometry. Each data point represents a biological replicate. For each group, the horizontal line represents the mean and the error bars represent the standard deviation of the mean. As described in the results presented in FIGS. 17A, at an RNP concentration of 700 mM, sucrose increased editing for each of the RNPs that were tested (see FIG. 17A, 0.5 M sucrose and 350 mM NaCl+0.5 M sucrose conditions). Thus, at a 700 mM concentration of TAGE, sucrose impacted T cell editing by TAGE independent of whether the TAGE included a cell-targeting moiety such as a T cell specific antibody. At a lower concentration (70 nM) of RNP, the impact of sucrose was more specific to TAGEs containing a T cell targeting antibody, as shown in FIG. 17B. As described in FIG. 17B, sucrose increased editing in T cell specific TAGE (TAGE with antibody AB2 or AB5) versus non-specific TAGE. The combination of both NaCl and sucrose further increased editing in non-specific antibody-containing TAGE AB1(NT)-TAGE26, as well as TAGE26 which did not contain an antibody. Thus, the combination of NaCl and sucrose, or even sucrose alone, as additives can improve editing by RNPs containing TAGE. Further, sucrose as an additive can improve editing specifically with a TAGE containing a cell-targeting antibody.

### Example 18: Inhibition of the Transporter NHE1 by Amiloride Derivatives Increased Editing of Primary Human T Cells by TAGE

In this example, the impact of the inhibition of the transporter NHE1 by amiloride derivatives on editing of primary human T cells by TAGE was assessed.

Antibodies were conjugated to Cas9 using the SpyTag2/SpyCatcher system and a guide RNA targeting CD47 was associated with the respective TAGE agents to form RNPs. Primary human T cells were pre-incubated with the indicated drug for 30 minutes. Then, RNPs were added to the cells at the indicated concentration and co-incubated for an additional hour. The cells were washed after one hour to remove inhibitors and RNPs. The percentage of cells that were edited under each condition was measured using a phenotypic readout detecting the loss of surface CD47 by flow cytometry.

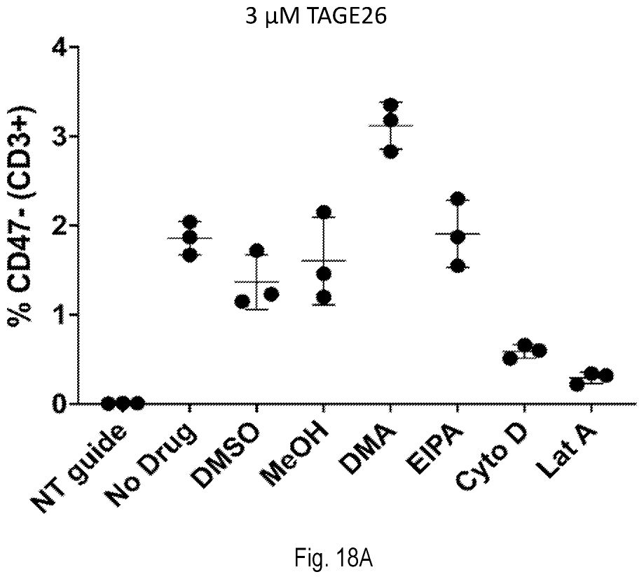

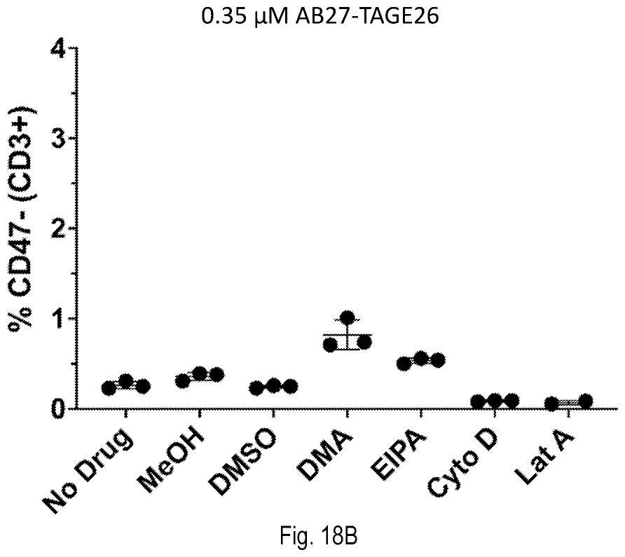

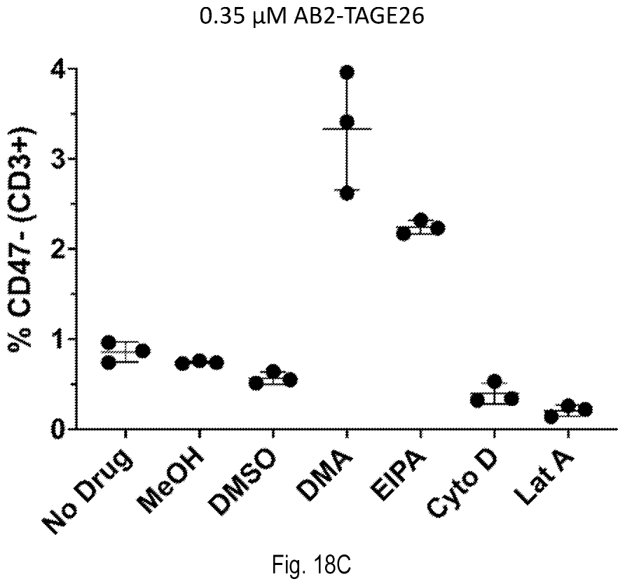

Data is presented in FIG. 18. Each data point represents a biological replicate. For each group, the horizontal line represents the mean and the error bars represent the standard deviation of the mean. With respect to the descriptions in FIG. 18, NT guide is a TAGE with non-targeting guide RNA; MeOH is methanol (a control for DMA and EIPA; used at 0.5% v/v; DMSO is dimethyl sulfoxide, a control for Cyto D and Lat A (used at 1% v/v); DMA is 5-(N,N-dimethyl)-amiloride, an inhibitor of NHE1 (used at 100 μM); EIPA is 5-(N-ethyl-N-isopropyl)-amiloride, an inhibitor of NHE1, (used at 100 μM); Cyto D is cytochalasin D, an inhibitor of F-actin polymerization (used at 20 μM); and Lat A is latrunculin A, an inhibitor of F-actin polymerization (used at 10 μM).

FIGS. 18A-18C provide data showing that editing of primary T cells by TAGE was increased by treatment with EIPA or DMA, which inhibited the transporter NHE1. FIGS. 18A-18C additionally provide data showing that editing was decreased by inhibitors of F-actin polymerization (see Cyto D, and Lat A). These results suggests that the effects of EIPA and DMA are not due to inhibition of macropinocytosis, which requires F-actin polymerization.

### Example 19: Nystatin Additive Increases Editing of T Cells by Cell Specific TAGE

In this example, the impact of the inhibition of Nystatin treatment on editing of primary human T cells by TAGE was assessed.

Antibodies were conjugated to Cas9 using the SpyTag/SpyCatcher system and a guide RNA targeting CD47 was associated with the respective TAGE agents to form an RNP. Primary human T cells were pre-incubated with the indicated drug (nystatin or Dynasore) for 30 minutes. Then, RNPs were added to cells at the indicated concentration and co-incubated for an additional hour. The cells were washed after one hour to remove inhibitors and RNPs. The percentage of cells that were edited under each condition was measured using a phenotypic readout detecting the loss of surface CD47 by flow cytometry. Each data point represents a biological replicate. For each group, the horizontal line represents the mean and the error bars represent the standard deviation of the mean. NT guide stood for TAGE with non-targeting guide RNA. DMSO is dimethylsulfoxide, a control for nystatin and Dynasore, which was used at 2% v/v. Dynasore is an inhibitor of dynamin, which was used at 80 μM. Nystatin was used at 108 μM.

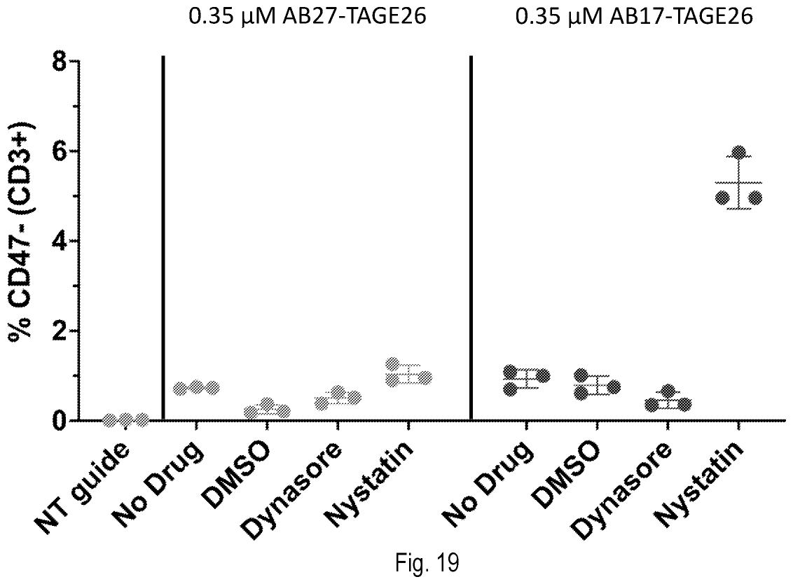

FIG. 19 concluded that editing of primary T cells by an antibody TAGE was increased by treatment with the drug nystatin. Nystatin did not increase editing by a TAGE containing a non-targeting antibody (AB27-TAGE26). In addition, FIG. 19 further provides data showing that editing was not increased by the drug dynasore, which inhibited dynamin activity. This suggests that the effect of nystatin is not due to inhibition of caveolin-mediated endocytosis, which requires dynamin activity.

### Example 20: Sucrose Additive Enables Editing at Lower Concentrations of a TAGE with CPP

In this example, the impact of sucrose on the editing of CPP-TAGE at lower concentrations was assessed.

A guide RNA targeting CD47 was associated with the CPP-TAGE Cas9-2×NLS-SpyCatcher-4×NLS to form a ribonucleoprotein (RNP). RNP at the indicated concentration was co-incubated with primary human T cells for 1 hour in T cell media with no additive (Baseline) or in T cell media with 0.5 M sucrose. The cells were washed after one hour to remove additives and RNPs. The percentage of cells that were edited under each condition was measured using a phenotypic readout detecting the loss of surface CD47 by flow cytometry. Each data point represents a biological replicate. For each group, the horizontal line represents the mean and the error bars represent the standard deviation of the mean.

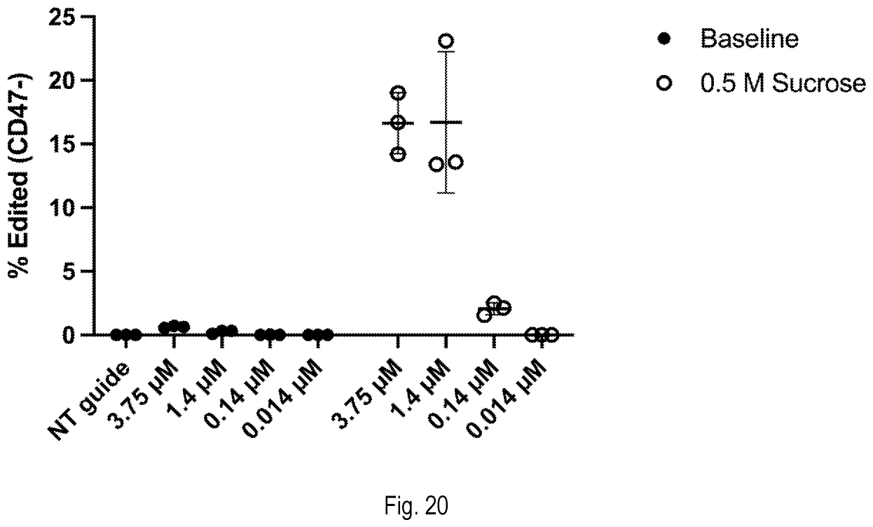

FIG. 20 provides data showing that treatment with 0.5 M sucrose increases editing of primary human T cells using a CPP-TAGE, and that treatment with 0.5 M sucrose enables editing of primary human concentrations with a lower concentration of CPP-TAGE.

The complete disclosure of all patents, patent applications, and publications, and electronically available material (including, for example, nucleotide sequence submissions in, e.g., GenBank and RefSeq. and amino acid sequence submissions in, e.g., SwissProt, PIR, PRF, PBD, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A stable aqueous formulation comprising:
at least 100 mM of a salt,
at least 3% w/v of a sugar or a sugar alcohol, and
a site-directed modifying polypeptide that recognizes a nucleic acid,
wherein the stable aqueous formulation has a pH of about 5 to 8.

2. The stable aqueous formulation of claim 1, wherein the formulation
comprises a reduced level of aggregates of the site-directed modifying polypeptide relative to a reference level as detected by UV/Vis absorbance spectroscopy;
remains stable after being subjected to at least two freeze-thaw cycles;
is stable during storage at about 4° C. for at least about 4 weeks, at least about 3 months, at least about 6 months, at least about 9 months, or at least about 1 year; and/or
is stable during storage at about 22° C. for at least about 4 weeks, at least about 3 months, at least about 6 months, at least about 9 months, or at least about 1 year.

3. The stable aqueous formulation of claim 2, wherein the reference level is the level of aggregates of the site-directed modifying polypeptide in phosphate buffered solution (PBS) as determined by size exclusion chromatography (SEC).

4. The stable aqueous formulation of claim 1, wherein:
the sugar is sucrose or trehalose;
the sugar alcohol is selected from the group consisting of glycerol, erythritol, xylitol, sorbitol, mannitol, and inositol; and/or
the salt is NaCl or KCl.

5. (canceled)

6. The stable aqueous formulation of claim 1, comprising:
at least about 1% w/v, at least about 2% w/v, at least about 3% w/v, at least about 4% w/v, at least about 5% w/v, at least about 7.5% w/v, at least about 10% w/v, at least about 12.5% w/v, at least about 15% w/v, at least about 20% w/v, at least about 25% w/v, at least about 30% w/v, at least about 35% w/v, at least about 40% w/v, at least about 45% w/v, at least about 50% w/v, or 1%-50% w/v of the sugar alcohol or the sugar;
at least about 50 mM, at least about 100 mM, at least about 150 mM, at least about 175 mM, at least about 185 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 500 mM, at least about 750 mM, at least about 1000 mM, at least about 1225 mM, at least about 1500 mM, at least about 1750 mM, at least about 2000 mM, or 50 mM to 2000 mM of the salt; and/or
0.1-100 μM of the site-directed modifying polypeptide.

7-12. (canceled)

13. The stable aqueous formulation of claim 1, wherein the site-directed modifying polypeptide that recognizes a nucleic acid is an RNA-guided nuclease.

14. The stable aqueous formulation of claim 13, wherein the RNA-guided nuclease is a Class 2 Cas polypeptide.

15. The stable aqueous formulation of claim 14, wherein the Class 2 Cas polypeptide is a Type II Cas polypeptide or a Type V Cas polypeptide.

16. The stable aqueous formulation of claim 15, wherein the Type II Cas polypeptide is Cas9 or wherein the Type V Cas polypeptide is Cas12.

17. The stable aqueous formulation of claim 13, further comprising a guide ribonucleic acid (gRNA), wherein the gRNA NA and the RNA-guided nuclease form a ribonucleoprotein (RNP).

18. (canceled)

19. The stable aqueous formulation of claim 17, wherein the gRNA is a re-folded gRNA that is a capable of eluting as a single peak from a Size Exclusion Chromatography resin when the gRNA is not complexed to the site-directed modifying polypeptide.

20. The stable aqueous formulation of claim 1, wherein the site-directed modifying polypeptide further comprises a cell targeting agent, thereby forming a targeted active gene editing (TAGE) agent.

21. The stable aqueous formulation of claim 20, wherein the cell targeting agent is a ligand, a cell penetrating peptide, or an antigen-binding polypeptide.

22. (canceled)

23. A method of modifying a nucleic acid in a target cell, the method comprising contacting the target cell with the stable aqueous formulation of claim 1.

24-48. (canceled)

49. A method for modifying a nucleic acid in a cell ex vivo, the method comprising contacting a cell in a cell medium with a targeted active gene editing agent (TAGE) that recognizes a nucleic acid in the cell,
wherein the TAGE comprises a cell targeting agent and a site-directed modifying polypeptide, and
wherein the cell medium comprises an effective amount of a salt and/or a sugar alcohol and/or a sugar, such that the nucleic acid in the cell is modified.

50. (canceled)

51. The method of claim 49, wherein the TAGE further comprises a guide RNA (gRNA).

52. (canceled)

53. The method of claim 49, wherein the cell targeting agent is a ligand, a cell penetrating peptide (CPP), an antigen-binding polypeptide, or a combination thereof.

54. (canceled)

55. The method of claim 49, wherein the site-directed modifying polypeptide is an RNA-guided nuclease.

56. The method of claim 55, wherein the RNA-guided nuclease is a Class 2 Cas polypeptide.

57. The method of claim 56, wherein the Class 2 Cas polypeptide is a Type II Cas polypeptide or a Type V Cas polypeptide.

58. The method of claim 57, wherein the Type II Cas polypeptide is Cas9 or the Type V Cas polypeptide is Cas 12.

59. (canceled)

60. The method of claim 49, wherein
the sugar alcohol is selected from the group consisting of erythritol, xylitol, mannitol, glycerol, and inositol;
the sugar is sucrose; and/or
the salt is NaCl or KCl.

61-66. (canceled)

67. The method of claim 49, wherein the mammalian cell is a mouse cell, a non-human primate cell, or a human cell.

68-73. (canceled)


SEQUENCE LISTING (4 sequences)

SEQ ID NO: 1 - protein, 9 aa, synthetic construct
LAGLIDADG

SEQ ID NO: 2 - protein, 9 aa, Human immunodeficiency virus 1
RKKRRQRRR

SEQ ID NO: 3 - protein, 12 aa, Human immunodeficiency virus 1
RQARRNRRRRWR

SEQ ID NO: 4 - protein, 7 aa, Simian Virus 40
PKKKRKV